(19) United States
(12) Patent Application Publication
(10) Pub. No.: US 2023/0192618 A1
(43) Pub. Date: Jun. 22, 2023

(54) CRYSTALLINE SALT FORMS OF N-(4-(4-(CYCLOPROPYLMETHYL)PIPERAZINE-1-CARBONYL)PHENYL)QUINOLINE-8-SULFONAMIDE

(71) Applicant: Agios Pharmaceuticals, Inc., Cambridge, MA (US)

(72) Inventor: Lisa M. Grove, Lafayette, IN (US)

(21) Appl. No.: 17/612,554

(22) PCT Filed: May 21, 2020
(86) PCT No.: PCT/US2020/033992

§ 371 Date: Nov. 18, 2021

Related U.S. Application Data
(60) Provisional application No. 62/851,344, filed on May 22, 2019.

(51) Int. Cl. C07D 215/36

(52) U.S. Cl. CPC C07D 215/36; C07B 2200/13

(57) ABSTRACT

Provided herein are various crystalline salt forms of compound (I) represented by the following structural formula: Also provided are pharmaceutical compositions comprising the crystalline salt forms, methods for their manufacture, and uses thereof for treating conditions associated with pyruvate kinase such as e.g., pyruvate kinase deficiency.

### RELATED APPLICATIONS

This application claims the benefit of U.S. Provisional Application No. 62/851,344, filed May 22, 2019, the entire contents of which are incorporated herein by reference.

### BACKGROUND

Pyruvate kinase deficiency (PKD) is a disease of the red blood cells caused by a deficiency of the pyruvate kinase R (PKR) enzyme due to recessive mutations of PKLR gene (Wijk et al. Human Mutation, 2008, 30 (3) 446-453). PKR activators can be beneficial to treat PKD, thalassemia (e.g., beta-thalessemia), abetalipoproteinemia or Bassen-Komzweig syndrome, sickle cell disease, paroxysmal nocturnal hemoglobinuria, anemia (e.g., congenital anemias (e.g., enzymopathies), hemolytic anemia (e.g. hereditary and/or congenital hemolytic anemia, acquired hemolytic anemia, chronic hemolytic anemia caused by phosphoglycerate kinase deficiency, anemia of chronic diseases, non-spherocytic hemolytic anemia or hereditary spherocytosis). Treatment of PKD is supportive, including blood transfusions, splenectomy, chelation therapy to address iron overload, and/or interventions for other disease-related morbidity. Currently, however, there is no approved medicine that treats the underlying cause of PKD, and thus the etiology of life-long hemolytic anemia.

N-(4-(4-(cyclopropylmethyl)piperazine-1-carbonyl)phenyl)quinoline-8-sulfonamide, herein referred to as Compound (I), is an allosteric activator of red cell isoform of pyruvate kinase (PKR). See e.g., WO 2011/002817 and WO 2016/201227, the contents of which are incorporated herein by reference.

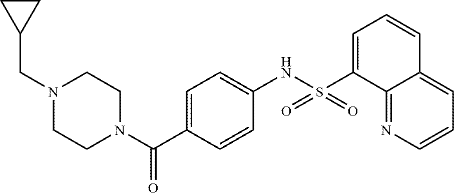 [SMILES: O=C(c1ccc(NS(=O)(=O)c2cccc3cccnc23)cc1)N1CCN(CC2CC2)CC1]

Compound (I) was developed to treat PKD and is currently being investigated in phase 2 clinical trials. See e.g., U.S. clinical trials identifier NCT02476916. Given its therapeutic benefits, there is a need to develop alternative forms of Compound (I) in an effort to facilitate isolation, manufacturing, and formulation development, as well as to enhance storage stability. In this context, amorphous and crystalline hemisulfate salt forms of Compound (I) are exemplified in International Application No. PCT/US2018/062197, the contents of which are incorporated herein by reference. The present invention further discloses alternative crystalline salt forms of Compound (I).

### SUMMARY

Provided herein is a crystalline besylate salt form of Compound (I) referred to as Form A.

Also provided are the crystalline fumarate salt forms of Compound (I) referred to as Form B and Form C.

Also provided are the crystalline gentisate salt forms of Compound (I) referred to as Form D and Form E.

Also provided are the crystalline hydrochloride salt forms of Compound (I) referred to as Form F and Form G.

Also provided is a crystalline maleate salt form of Compound (I) referred to as Form H.

Also provided is a crystalline malonate salt form of Compound (I) referred to as Form I.

Also provided are crystalline phosphate salt forms of Compound (I) referred to as Form J and Form K.

Also provided is a crystalline tartrate salt form of Compound (I) referred to as Form L.

Also provided is a crystalline tosylate salt form of Compound (I) referred to as Form M.

Also provided herein are pharmaceutical compositions comprising the crystalline salt Form A, B, C, D, E, F, G, H, I, J, K, L, or M, methods for their manufacture, and uses thereof for treating conditions associated with pyruvate kinase such as e.g., PKD.

### BRIEF DESCRIPTION OF THE FIGURES

FIG. 1 depicts an X-ray powder diffraction pattern (XRPD) for crystalline besylate salt Form A.

FIG. 2 depicts the combined thermogravimetric analysis (TGA) thermogram and differential scanning calorimetry (DSC) thermogram for crystalline besylate salt Form A.

FIG. 3 depicts an X-ray powder diffraction pattern (XRPD) for crystalline fumarate salt Form B.

FIG. 4 depicts the combined thermogravimetric analysis (TGA) thermogram and differential scanning calorimetry (DSC) thermogram for crystalline fumarate salt Form B.

FIG. 5 depicts an X-ray powder diffraction pattern (XRPD) for crystalline fumarate salt Form C.

FIG. 6 depicts the combined thermogravimetric analysis (TGA) thermogram and differential scanning calorimetry (DSC) thermogram for crystalline fumarate salt Form C.

FIG. 7 depicts an X-ray powder diffraction pattern (XRPD) for crystalline gentisate salt Form D.

FIG. 8 depicts the combined thermogravimetric analysis (TGA) thermogram and differential scanning calorimetry (DSC) thermogram for crystalline gentisate salt Form D.

FIG. 9 depicts an X-ray powder diffraction pattern (XRPD) for crystalline gentisate salt Form E.

FIG. 10 depicts the combined thermogravimetric analysis (TGA) thermogram and differential scanning calorimetry (DSC) thermogram for crystalline gentisate salt Form E.

FIG. 11 depicts an X-ray powder diffraction pattern (XRPD) for crystalline hydrochloride salt Form F.

FIG. 12 depicts the combined thermogravimetric analysis (TGA) thermogram and differential scanning calorimetry (DSC) thermogram for crystalline hydrochloride salt Form F.

FIG. 13 depicts an X-ray powder diffraction pattern (XRPD) for crystalline hydrochloride salt Form G.

FIG. 14 depicts the combined thermogravimetric analysis (TGA) thermogram and differential scanning calorimetry (DSC) thermogram for crystalline hydrochloride salt Form G.

FIG. 15 depicts an X-ray powder diffraction pattern (XRPD) for crystalline maleate salt Form H.

FIG. 16 depicts the combined thermogravimetric analysis (TGA) thermogram and differential scanning calorimetry (DSC) thermogram for crystalline maleate salt Form H.

FIG. 17 depicts an X-ray powder diffraction pattern (XRPD) for crystalline malonate salt Form I.

FIG. 18 depicts the combined thermogravimetric analysis (TGA) thermogram and differential scanning calorimetry (DSC) thermogram for crystalline malonate salt Form I.

FIG. 19 depicts an X-ray powder diffraction pattern (XRPD) for crystalline phosphate salt Form J.

FIG. 20 depicts the combined thermogravimetric analysis (TGA) thermogram and differential scanning calorimetry (DSC) thermogram for crystalline phosphate salt Form J.

FIG. 21 depicts an X-ray powder diffraction pattern (XRPD) for crystalline phosphate salt Form K.

FIG. 22 depicts the combined thermogravimetric analysis (TGA) thermogram and differential scanning calorimetry (DSC) thermogram for crystalline phosphate salt Form K.

FIG. 23 depicts an X-ray powder diffraction pattern (XRPD) for crystalline tartrate salt Form L.

FIG. 24 depicts the combined thermogravimetric analysis (TGA) thermogram and differential scanning calorimetry (DSC) thermogram for crystalline tartrate salt Form L.

FIG. 25 depicts an X-ray powder diffraction pattern (XRPD) for crystalline tosylate salt Form M.

FIG. 26 depicts the combined thermogravimetric analysis (TGA) thermogram and differential scanning calorimetry (DSC) thermogram for crystalline tosylate salt Form M.

### DETAILED DESCRIPTION

### Definitions

As used herein, “crystalline” refers to a solid form of a compound wherein there exists long-range atomic order in the positions of the atoms. The crystalline nature of a solid can be confirmed, for example, by examination of the X-ray powder diffraction pattern. If the XRPD shows sharp intensity peaks in the XRPD then the compound is crystalline.

When used alone, the terms “Form A”, “Form B”, “Form C”, “Form D”, “Form E”, “Form F”, “Form G”, “Form H”, “Form I”, “Form J”, “Form L”, and “Form M”, refer to the crystalline salt forms A1, B, C, D, E, F, G, H, I, J, L, and M of Compound (I), respectively. The terms “Form A”, “crystalline Form A”, and “crystalline besylate salt Form A of Compound (I)” are used interchangeably. Similarly, “Form B”, “crystalline Form B”, and “crystalline fumarate salt Form B of Compound (I)” are used interchangeably. Similarly, “Form C”, “crystalline Form C”, and “crystalline fumarate salt Form C of Compound (I)” are used interchangeably. Similarly, “Form D”, “crystalline Form D”, and “crystalline gentisate salt Form D of Compound (I)” are used interchangeably. Similarly, “Form E”, “crystalline Form E”, and “crystalline gentisate salt Form E of Compound (I)” are used interchangeably. Similarly, “Form F”, “crystalline Form F”, and “crystalline hydrochloride salt Form F of Compound (I)” are used interchangeably. Similarly, “Form G”, “crystalline Form G”, and “crystalline hydrochloride salt Form G of Compound (I)” are used interchangeably. Similarly, “Form H”, “crystalline Form H”, and “crystalline maleate salt Form H of Compound (I)” are used interchangeably. Similarly, “Form I”, “crystalline Form I”, and “crystalline malonate salt Form I of Compound (I)” are used interchangeably. Similarly, “Form J”, and “crystalline Form J”, “crystalline phosphate salt Form J of Compound (I)” are used interchangeably. Similarly, “Form K”, and “crystalline Form K”, “crystalline phosphate salt Form K of Compound (I)” are used interchangeably. Similarly, “Form L”, and “crystalline Form L”, “crystalline tartrate salt Form L of Compound (I)” are used interchangeably. Similarly, “Form M”, and “crystalline Form M”, “crystalline tosylate salt Form M of Compound (I)” are used interchangeably.

Unless otherwise specified, for any given salt of compound (I), the crystalline salt form(s) of Compound (I) are each single crystalline forms. A “single crystalline form” means that the recited crystalline salt form of Compound (I), is present as a single crystal or a plurality of crystals in which each crystal has the same crystal form. Percent by weight of a particular crystal form is determined by the weight of the particular crystal form divided by the sum weight of the particular crystal, plus the weight of the other crystal forms present plus the weight of amorphous form present multiplied by 100%.

Chemical purity refers to extent by which the disclosed form is free from materials having different chemical structures. Chemical purity of the compound in the disclosed crystal forms means the weight of the compound divided by the sum of the weight of the compound plus materials/impurities having different chemical structures multiplied by 100%, i.e., percent by weight.

The terms “anhydrous” and “anhydrate” are used interchangeably and mean that the referenced crystalline form has substantially no water in the crystal lattice, e.g., less than 1.5% by weight as determined by Karl Fisher analysis.

The term “solvate” refers to a crystalline compound wherein a stoichiometric or non-stoichiometric amount of solvent, or mixture of solvents, is incorporated into the crystal structure.

The term “hydrate” refers to a crystalline compound where a stoichiometric or non- stoichiometric amount of water is incorporated into the crystal structure. A hydrate is a solvate wherein the solvent incorporated into the crystal structure is water. The term “anhydrous” when used with respect to a compound means substantially no solvent incorporated into the crystal structure.

The term “amorphous” means a solid that is present in a non-crystalline state or form. Amorphous solids are disordered arrangements of molecules and therefore possess no distinguishable crystal lattice or unit cell and consequently have no definable long range ordering. Solid state ordering of solids may be determined by standard techniques known in the art, e.g., by X-ray powder diffraction (XRPD) or differential scanning calorimetry (DSC). Amorphous solids can also be differentiated from crystalline solids e.g., by birefringence using polarized light microscopy.

The 2-theta values of the X-ray powder diffraction patterns for the crystalline forms described herein may vary slightly from one instrument to another and also depending on variations in sample preparation and batch to batch variation due to factors such as temperature variation, sample displacement, and the presence or absence of an internal standard. Therefore, unless otherwise defined, the XRPD patterns / assignments recited herein are not to be construed as absolute and can vary ± 0.2 degrees. It is well known in the art that this variability will account for the above factors without hindering the unequivocal identification of a crystal form. Unless otherwise specified, the 2-theta values provided herein were obtained using Cu Kα1 radiation.

“Substantially the same XRPD pattern” or “an X-ray powder diffraction pattern substantially similar to” a defined figure means that for comparison purposes, at least 90% of the peaks shown are present. It is to be further understood that for comparison purposes some variability in peak intensities from those shown are allowed, such as ± 5% of the intensity of the most intense peak.

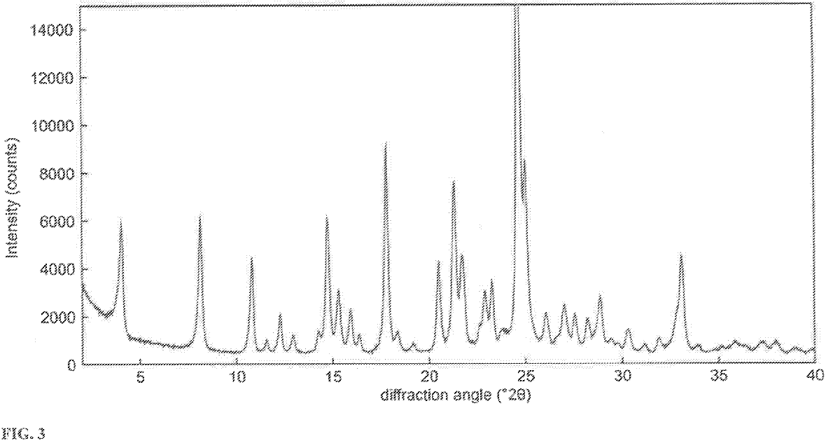

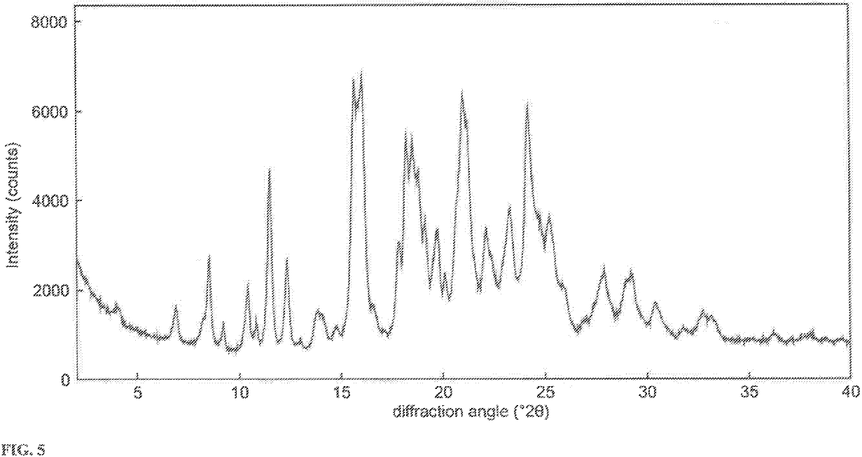

The amount of one crystalline form relative to another crystalline form in a sample can be assessed by preparing a series of mixtures of the two crystalline forms with known weight ratios and obtaining an XRPD spectrum for each. For example, the relative amounts of crystalline fumarate salt Form B and Form C in a sample can be assessed by selecting one or more characteristic peaks of crystalline Form B and Form C depicted in FIG. 3 and FIG. 5, respectively, and correlating their relative intensities in the sample XRPD to their relative intensities in the mixture XRPDs.

Temperature values, e.g., for DSC peaks herein may vary slightly from one instrument to another and also depending on variations in sample preparation, batch to batch variation, and environmental factors. Therefore, unless otherwise defined, temperature values recited herein are not to be construed as absolute and can vary ± 5 degrees or ± 2 degrees.

The terms “ambient temperature” and “room temperature” are used interchangeably and refer to the range of air temperatures relating to the immediate surroundings, that is, between 20 to 25° C. (68 to 77° F.), with excursions between 15 to 30° C. (59 to 86° F.) allowed, provided the mean kinetic temperature does not exceed 25° C. (77° F.), by following the guideline of the United States Pharmacopeia-National Formulary (USP-NF).

An “effective amount” of a compound described herein is an amount sufficient to provide a therapeutic benefit in the treatment of a condition or to delay or minimize one or more symptoms associated with the condition. The terms “effective amount” and “therapeutically effective amount” are used interchangeably. In one aspect, an effective amount of a compound means an amount of therapeutic agent, alone or in combination with other therapies, which provides a therapeutic benefit in the treatment of the condition. The term “effective amount” can encompass an amount that improves overall therapy, reduces or avoids symptoms, signs, or causes of the condition, and/or enhances the therapeutic efficacy of another therapeutic agent. In certain embodiments, an effective amount is an amount sufficient for eliciting measurable activation of wild-type or mutant PKR. In certain embodiments, an effective amount is an amount sufficient for regulating 2,3-diphosphoglycerate levels in blood in need thereof or for treating pyruvate kinase deficiency (PKD), hemolytic anemia (e.g., chronic hemolytic anemia, hereditary non-spherocytic anemia), sickle cell disease, thalassemia (e.g., alfa thalassemia, beta-thalassemia or non-transfusion-dependent thalassemia), hereditary spherocytosis, hereditary elliptocytosis, abetalipoproteinemia (or Bassen-Komzweig syndrome), paroxysmal nocturnal hemoglobinuria, acquired hemolytic anemia (e.g., congenital anemias (e.g., enzymopathies)), anemia of chronic diseases or treating diseases or conditions that are associated with increased 2,3-diphosphoglycerate levels (e.g., liver diseases). In certain embodiments, an effective amount is an amount sufficient for eliciting measurable activation of wild-type or mutant PKR and for regulating 2,3-diphosphoglycerate levels in blood in need thereof or for treating pyruvate kinase deficiency (PKD), hemolytic anemia (e.g., chronic hemolytic anemia, hereditary non-spherocytic anemia), sickle cell disease, thalassemia (e.g., alfa thalassemia, beta-thalassemia or non-transfusion-dependent thalassemia), hereditary spherocytosis, hereditary elliptocytosis, abetalipoproteinemia (or Bassen-Komzweig syndrome), paroxysmal nocturnal hemoglobinuria, acquired hemolytic anemia (e.g., congenital anemias (e.g., enzymopathies)), anemia of chronic diseases or treating diseases or conditions that are associated with increased 2,3-diphosphoglycerate levels (e.g., liver diseases). In one aspect, the effective amount is the amount required to generate a subject’s hemoglobin response of ≥1.0 g/dL (such as ≥1.5 g/dL or ≥2.0 g/dL) increase in Hb concentration from baseline. In one aspect, the subject’s baseline Hb concentration is the average of all available Hb concentrations before treatment with a compound described herein. In certain aspects, the effective amount is the amount required to reduce the patient’s transfusion burden. In one aspect, the effective amount is between 0.01 - 100 mg/kg body weight/day of the provided compound, such as e.g., 0.1 - 100 mg/kg body weight/day.

As used herein, reduction in transfusion burden means at least 20% reduction in the number of RBC units transfused within at least 5 weeks of treatment. In certain embodiments, the reduction in transfusion burden is ≥33% reduction in the number of RBC units transfused within at least 5 weeks of treatment. In certain embodiments, reduction of transfusion burden is ≥33% reduction in the number of RBC units transfused within at least 10 weeks (e.g., at least 20 weeks or at least 24 weeks) of treatment.

As used herein, sickle cell disease (SCD), Hemoglobin SS disease, and sickle cell anemia are used interchangeably. Sickle cell disease (SCD) is an inherited blood disorder caused by the presence of sickle hemoglobin (HbS). In certain embodiments, subjects with SCD have abnormal hemoglobin, called hemoglobin S or sickle hemoglobin, in their red blood cells. In certain embodiments, people having SCD have at least one abnormal genes causing the body to make hemoglobin S. In certain embodiments, people having SCD have two hemoglobin S genes, Hemoglobin SS.

Thalassemia is an inherited blood disorder in which the normal ratio of α- to β-globin production is disrupted due to a disease-causing variant in 1 or more of the globin genes. In certain embodiments, Alpha-globin aggregates (as found in β-thalassemia) readily precipitate, which disrupts the red blood cell (RBC) membrane and results in oxidative stress. In certain embodiments, Beta-globin tetramers (Hb H, found in α-thalassemia) are generally more soluble, but are still unstable and can form precipitates. The imbalance of the globin chain synthesis can lead to a net reduction in Hb concentrations and has dramatic effects on the survival of RBC precursors, ultimately resulting in their premature destruction in the bone marrow and in extramedullary sites (Cappellini et al, 2014). In certain embodiments, the disorder results in large numbers of red blood cells being destroyed, which leads to anemia. In certain embodiments, the thalassemia is alpha thalassemia. In certain embodiments, the thalassemia is beta thalassemia. In other embodiments, the thalassemia is non-transfusion-dependent thalassemia. In other embodiments, the thalassemia is beta thalassemia intermedia. In other embodiments, the thalassemia is Hb E beta thalassemia. In other embodiments, the thalassemia is beta thalassemia with mutations of 1 or more alfa genes.

The term “activating” as used herein means an agent that (measurably) increases the activity of wild type pyruvate kinase R (wt PKR) or causes wild type pyruvate kinase R (wt PKR) activity to increase to a level that is greater than wt PKR’s basal levels of activity or an agent that (measurably) increases the activity of a mutant pyruvate kinase R (mPKR) or causes mutant pyruvate kinase R (mPKR) activity to increase to a level that is greater than that mutant PKR’s basal levels of activity, for examples, to a level that is 20%, 40%, 50%, 60%, 70%, 80%, 90% or 100% of the activity of wild type PKR.

The term “packed red blood cells” or PRBCs as used herein refer to red blood cells made from a unit of whole blood by centrifugation and removal of most of the plasma. In certain embodiments, a PRBC unit has a hematocrit of at least about 95%. In certain embodiments, a PRBC unit has a hematocrit of at least about 90%. In certain embodiments, a PRBC unit has a hematocrit of at least about 80%. In certain embodiments, a PRBC unit has a hematocrit of at least about 70%. In certain embodiments, a PRBC unit has a hematocrit of at least about 60%. In certain embodiments, a PRBC unit has a hematocrit of at least about 50%. In certain embodiments, a PRBC unit has a hematocrit of at least about 40%. In certain embodiments, a PRBC unit has a hematocrit of at least about 30%. In certain embodiments, a PRBC unit has a hematocrit of at least about 20%. In certain embodiments, a PRBC unit has a hematocrit of at least about 10%.

The terms “treatment,” “treat,” and “treating” refer to reversing, alleviating, reducing the likelihood of developing, or inhibiting the progress of a disease or disorder, or one or more symptoms thereof, as described herein. In some embodiments, treatment may be administered after one or more symptoms have developed, i.e., therapeutic treatment. In other embodiments, treatment may be administered in the absence of symptoms. For example, treatment may be administered to a susceptible individual prior to the onset of symptoms (e.g., in light of a history of symptoms and/or in light of genetic or other susceptibility factors), i.e., prophylactic treatment. Treatment may also be continued after symptoms have resolved, for example to reduce the likelihood of or delay their recurrence.

As used herein the terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment. In certain embodiments, the term “subject” refers to a human subject in need of treatment of a disease. In certain embodiments, the term “subject” refers to a human subject in need of treatment of PKD. In certain embodiments, the term “subject” refers to a human subject in need of treatment of thalassemia. In certain embodiments, the term “subject” refers to a human subject in need of treatment of sickle cell disease. In certain embodiments, the term “subject” refers to a human adult over 18 years old in need of treatment of a disease. In certain embodiments, the term “subject” refers to a human child no more than 18 years old in need of treatment of a disease. In certain embodiments, the subject is a patient in need of regular blood transfusion. As used here, the regular blood transfusion refers to at least 4 transfusion episodes in a 52-week period prior to the treatment. In certain embodiments, the regular blood transfusion refers to at least 5 transfusion episodes in a 52-week period prior to the treatment. In certain embodiments, the regular blood transfusion refers to at least 6 transfusion episodes in a 52-week period prior to the treatment. In certain embodiments, the regular blood transfusion refers to at least 7 transfusion episodes in a 52-week period prior to the treatment. In certain embodiments, the subject with a least one of the indications selected from the sickle cell disease, thalassemia, PKD under regular transfusion, and non-transfusion dependent PKD, has not been exposed to sotatercept (ACE-011), luspatercept (ACE-536), ruxolitinib, or gene therapy. In certain embodiments, such subject is not taking inhibitors of cytochrome P450 (CYP)3A4, strong inducers of CYP3A4, strong inhibitors of P-glycoprotein (P-gp), or digoxin. In certain embodiments, such subject is not receiving chronic anticoagulant therapy, anabolic steroids, hematopoietic stimulating agents (eg, erythropoietins, granulocyte colony stimulating factors, thrombopoietins), or allergic to sulfonamides.

The term “pharmaceutically acceptable carrier” refers to a non-toxic carrier, adjuvant, or vehicle that does not adversely affect the pharmacological activity of the compound with which it is formulated, and which is also safe for human use.

As used herein, the terms “about” and “approximately” when used in combination with a numeric value or range of values used to characterize a particular crystal form, amorphous form, or mixture thereof of a compound mean the value or range of values may deviate to an extent deemed reasonable to one of ordinary skill in the art while describing the particular crystal form, amorphous form, or mixture thereof.

### Exemplary Forms

Provided herein is a besylate salt of compound (I) represented by the following structural formula:

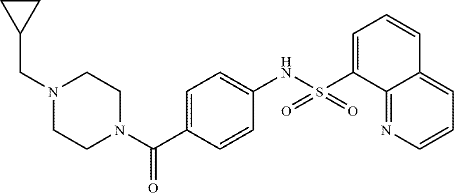 [SMILES: O=C(c1ccc(NS(=O)(=O)c2cccc3cccnc23)cc1)N1CCN(CC2CC2)CC1]

wherein the molar ratio between compound (I) and besylate acid is 1: 1.

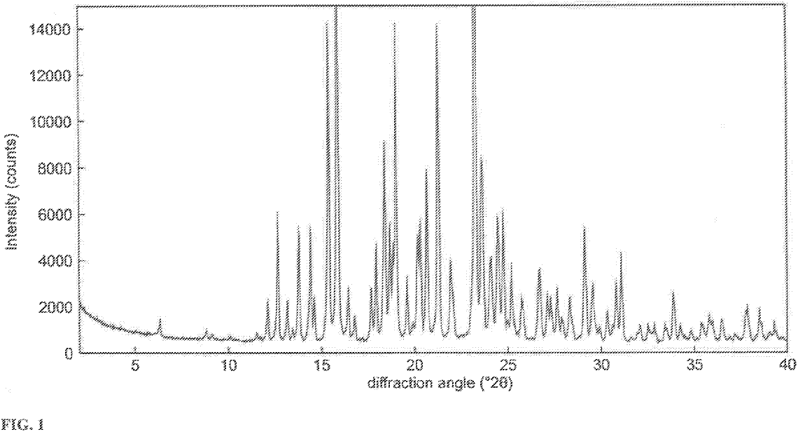

In one aspect, the besylate salt of compound (I) is a crystalline form. In one embodiment of the aspect, the besylate salt of compound (I) is anhydrous. In one specific embodiment, the crystalline form is crystalline Form A characterized by x-ray powder diffraction peaks at 2Θ angles (± 0.2°) 15.4°, 15.9°, 21.3°, and 23.3°. In another specific embodiment, crystalline Form A is characterized by x-ray powder diffraction peaks at 2Θ angles (± 0.2°) 15.4°, 15.9°, 21.3°, and 23.3°; and at least one, at least two, or at least three additional x-ray powder diffraction peaks at 2Θ angles (± 0.2°) selected from 18.4°, 19.0°, 20.7°, and 24.5°. In yet another specific embodiment, crystalline Form A is characterized by x-ray powder diffraction peaks at 2Θ angles (± 0.2°) 15.4°, 15.9°, 18.4°, 19.0°, 20.7°, 21.3°, 23.3°, and 24.5°. In yet another specific embodiment, crystalline Form A is characterized by x-ray powder diffraction peaks at 2Θ angles (± 0.2°) 12.7°, 14.5°, 15.4°, 15.9°, 18.4°, 19.0°, 20.7°, 21.3°, 23.3°, 23.6°, 24.1°, and 24.5°. In yet another specific embodiment, crystalline Form A is characterized by an X-ray powder diffraction pattern substantially similar to FIG. 1.

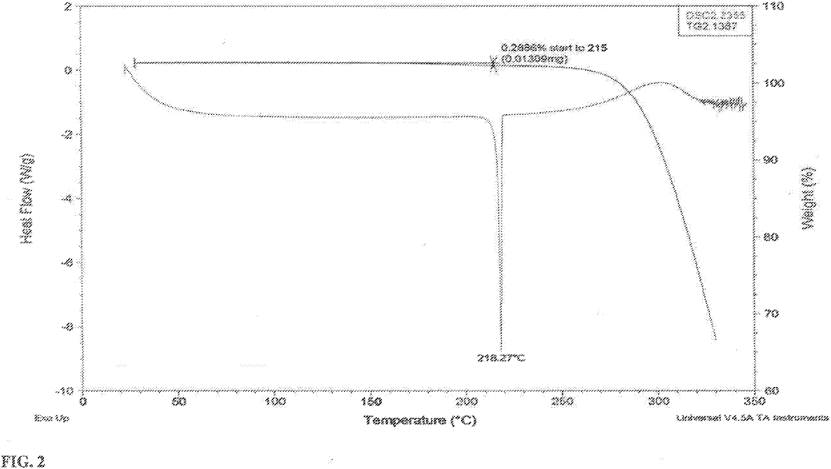

In one alternative specific embodiment, crystalline Form A is characterized by a Differential Scanning Calorimetry (DSC) with a sharp endotherm at 218.3° C. (onset temperature), or Thermogravimetric analysis (TGA) of a 0.3% weight loss between 20 and 215° C., or both, wherein the crystalline Form A may also comprise XRPD peaks at 2Θ angles selected from any of those described above. Alternatively, crystalline Form A is characterized by a Differential Scanning Calorimetry (DSC) or Thermogravimetric analysis (TGA) substantially similar to FIG. 2, wherein the crystalline Form A may also comprise XRPD peaks at 2Θ angles selected from any of those described above.

In another alternative, crystalline Form A as described in the above specific embodiments is at least 60% a single crystalline form, at least 70% a single crystalline form, at least 80% a single crystalline form, at least 90% a single crystalline form, at least 95% a single crystalline form, or at least 99% a single crystalline form by weight.

In yet another alternative, crystalline Form A as described in the above specific embodiments has a chemical purity of at least 60%, at least 70%, at least 80%, at least 90%, at least 95%, or at least 99% by weight.

Also provided herein is a fumarate salt of compound (I) represented by the following structural formula:

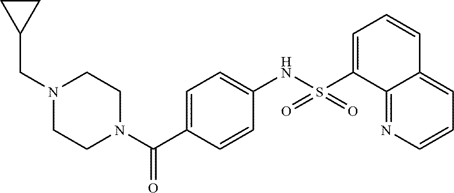 [SMILES: O=C(c1ccc(NS(=O)(=O)c2cccc3cccnc23)cc1)N1CCN(CC2CC2)CC1]

wherein the molar ratio between compound (I) and fumaric acid is 1: 1.

In one aspect, the fumarate salt of compound (I) is a crystalline form. In one specific aspect, the fumarate salt of compound (I) is a solvate. Further specified, the fumarate salt of compound (I) is a hydrate. In one embodiment, the crystalline form is crystalline Form B characterized by x-ray powder diffraction peaks at 2Θ angles (± 0.2°) 17.8°, 24.7°, 25.0°, and 33.1°. In another embodiment, crystalline Form B is characterized by x-ray powder diffraction peaks at 2Θ angles (± 0.2°) 17.8°, 24.7°, 25.0°, and 33.1°; and at least one, at least two, or at least three additional x-ray powder diffraction peaks at 2Θ angles (± 0.2°) selected from 4.1°, 8.2°, 14.8°, and 21.3°. In yet another embodiment, crystalline Form B is characterized by x-ray powder diffraction peaks at 2Θ angles (± 0.2°) 4.1°, 8.2°, 14.8°, 17.8°, 21.3°, 24.7°, 25.0°, and 33.1°. In yet another embodiment, crystalline Form B is characterized by x-ray powder diffraction peaks at 2Θ angles (± 0.2°) 4.1°, 8.2°, 10.8°, 14.8°, 15.3°, 17.8°, 20.5°, 21.3°, 21.7°, 24.7°, 25.0°, and 33.1°. In yet another embodiment, crystalline Form B is characterized by an X-ray powder diffraction pattern substantially similar to FIG. 3.

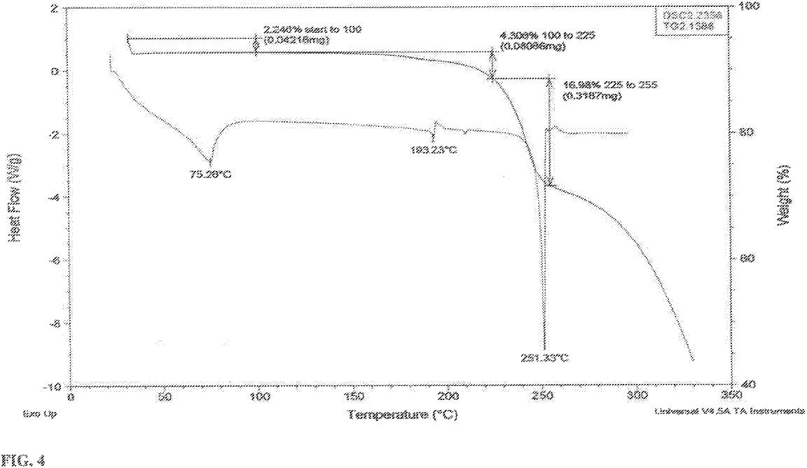

In one alternative embodiment, crystalline Form B is characterized by a Differential Scanning Calorimetry (DSC) with three endotherms at 75.3, 193.2 and 251.3° C. (onset temperatures), or Thermogravimetric analysis (TGA) of a 2.2% weight loss between 20 and 100° C. as well as a 4.3% weight loss between 100 and 225° C., or both, wherein the crystalline Form B may also comprise XRPD peaks at 2Θ angles selected from any of those described above. Alternatively, crystalline Form B is characterized by a Differential Scanning Calorimetry (DSC) or Thermogravimetric analysis (TGA) substantially similar to FIG. 4, wherein the crystalline Form B may also comprise XRPD peaks at 2Θ angles selected from any of those described above.

In another alternative, crystalline Form B as described in the above embodiments is at least 60% a single crystalline form, at least 70% a single crystalline form, at least 80% a single crystalline form, at least 90% a single crystalline form, at least 95% a single crystalline form, or at least 99% a single crystalline form by weight.

In yet another alternative, crystalline Form B as described in the above embodiments has a chemical purity of at least 60%, at least 70%, at least 80%, at least 90%, at least 95%, or at least 99% by weight.

Also provided herein is a fumarate salt of compound (I) represented by the following structural formula:

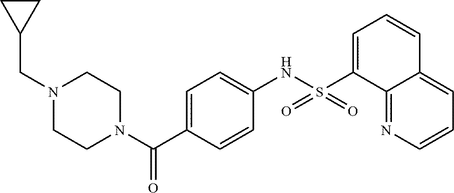 [SMILES: O=C(c1ccc(NS(=O)(=O)c2cccc3cccnc23)cc1)N1CCN(CC2CC2)CC1]

wherein the molar ratio between compound (I) and fumaric acid is 1: 1.

In one aspect, the fumarate salt of compound (I) is a crystalline form. In one embodiment of the aspect, the fumarate salt of compound (I) is anhydrous. In one specific embodiment, the crystalline form is crystalline Form C characterized by x-ray powder diffraction peaks at 2Θ angles (± 0.2°) 15.6°, 16.1°, 18.7°, and 25.2°. In another specific embodiment, crystalline Form C is characterized by x-ray powder diffraction peaks at 2Θ angles (± 0.2°) 15.6°, 16.1°, 18.7°, and 25.2°; and at least one, at least two, or at least three additional x-ray powder diffraction peaks at 2Θ angles (± 0.2°) selected from 11.5°, 18.2°, 21.3°, and 24.1°. In yet another specific embodiment, crystalline Form C is characterized by x-ray powder diffraction peaks at 2Θ angles (± 0.2°) 11.5°, 15.6°, 16.1°, 18.2°, 18.7°, 21.3°, 24.1 °, and 25.2°. In yet another specific embodiment, crystalline Form C is characterized by x-ray powder diffraction peaks at 2Θ angles (± 0.2°) 8.5°, 11.5°, 15.6°, 16.1°, 17.8°, 18.2°, 18.7°, 21.0°, 21.3°, 24.1°, 25.2°, 27.8°, and 29.1°. In yet another specific embodiment, crystalline Form C is characterized by an X-ray powder diffraction pattern substantially similar to FIG. 5.

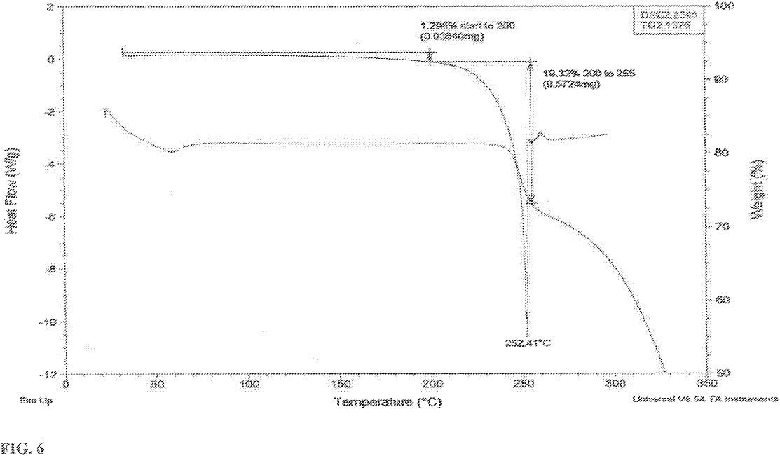

In one alternative specific embodiment, crystalline Form C is characterized by a Differential Scanning Calorimetry (DSC) with a sharp endotherm at 252.4° C. (onset temperature), or Thermogravimetric analysis (TGA) of a 1.3% weight loss between 20 and 200° C., or both, wherein the crystalline Form C may also comprise XRPD peaks at 2Θ angles selected from any of those described above. Alternatively, crystalline Form C is characterized by a Differential Scanning Calorimetry (DSC) or Thermogravimetric analysis (TGA) substantially similar to FIG. 6, wherein the crystalline Form C may also comprise XRPD peaks at 2Θ angles selected from any of those described above.

In another alternative, crystalline Form C as described in the above specific embodiments is at least 60% a single crystalline form, at least 70% a single crystalline form, at least 80% a single crystalline form, at least 90% a single crystalline form, at least 95% a single crystalline form, or at least 99% a single crystalline form by weight.

In yet another alternative, crystalline Form C as described in the above specific embodiments has a chemical purity of at least 60%, at least 70%, at least 80%, at least 90%, at least 95%, or at least 99% by weight.

Also provided herein is a gentisate salt of compound (I) represented by the following structural formula:

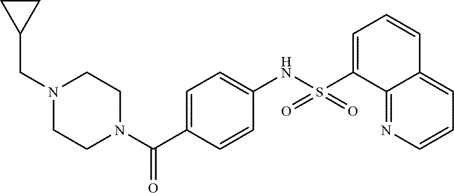 [SMILES: O=C(c1ccc(NS(=O)(=O)c2cccc3cccnc23)cc1)N1CCN(CC2CC2)CC1]

wherein the molar ratio between compound (I) and gentisic acid is 1: 1.

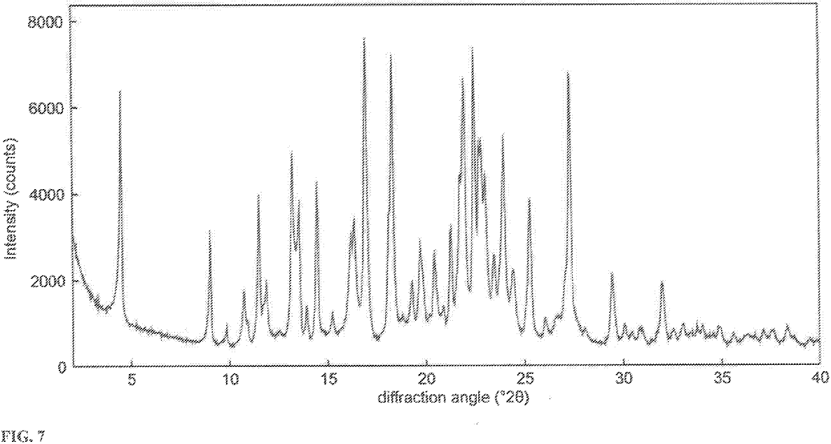

In one aspect, the gentisate salt of compound (I) is a crystalline form. In one embodiment of the aspect, the gentisate salt of compound (I) is anhydrous. In one specific embodiment, the crystalline form is crystalline Form D characterized by x-ray powder diffraction peaks at 2Θ angles (± 0.2°) 16.9°, 21.7°, 22.4°, and 23.9°. In another specific embodiment, crystalline Form D is characterized by x-ray powder diffraction peaks at 2Θ angles (± 0.2°) 16.9°, 21.7°, 22.4°, and 23.9°; and at least one, at least two, or at least three additional x-ray powder diffraction peaks at 2Θ angles (± 0.2°) selected from 4.5°, 13.2°, 16.1°, and 18.1°. In yet another specific embodiment, crystalline Form D is characterized by x-ray powder diffraction peaks at 2Θ angles (± 0.2°) 4.5°, 13.2°, 16.1°, 16.9°, 18.1°, 21.7°, 22.4°, and 23.9°. In yet another specific embodiment, crystalline Form D is characterized by x-ray powder diffraction peaks at 2Θ angles (± 0.2°) 4.5°, 13.2°, 13.6°, 16.1°, 16.9°, 18.1°, 21.7°, 22.4°, 23.0°, 23.9°, 27.1°, and 27.3°. In yet another specific embodiment, crystalline Form D is characterized by an X-ray powder diffraction pattern substantially similar to FIG. 7.

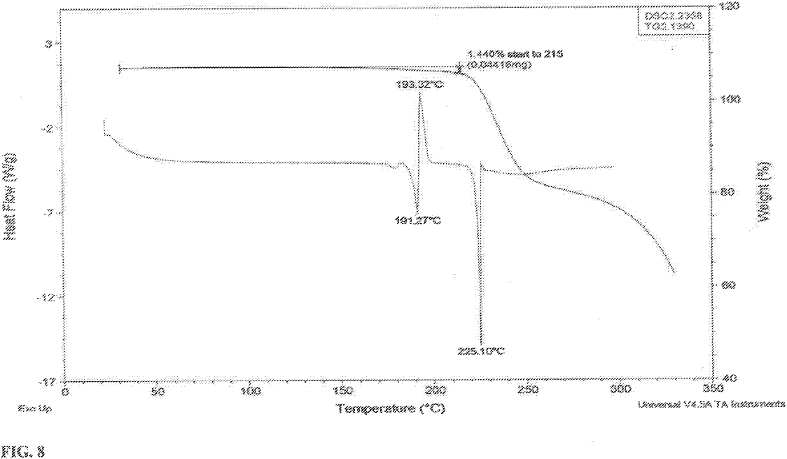

In one alternative specific embodiment, crystalline Form D is characterized by a Differential Scanning Calorimetry (DSC) with two endotherms at 191.3 and 225.1° C. (onset temperatures) and an exotherm at 193.3° C. (onset temperature), or Thermogravimetric analysis (TGA) of a 1.4% weight loss between 20 and 215° C., or both, wherein the crystalline Form D may also comprise XRPD peaks at 2Θ angles selected from any of those described above. Alternatively, crystalline Form D is characterized by a Differential Scanning Calorimetry (DSC) or Thermogravimetric analysis (TGA) substantially similar to FIG. 8, wherein the crystalline Form D may also comprise XRPD peaks at 2Θ angles selected from any of those described above.

In another alternative, crystalline Form D as described in the above specific embodiments is at least 60% a single crystalline form, at least 70% a single crystalline form, at least 80% a single crystalline form, at least 90% a single crystalline form, at least 95% a single crystalline form, or at least 99% a single crystalline form by weight.

In yet another alternative, crystalline Form D as described in the above specific embodiments has a chemical purity of at least 60%, at least 70%, at least 80%, at least 90%, at least 95%, or at least 99% by weight.

Also provided herein is a gentisate salt of compound (I) represented by the following structural formula:

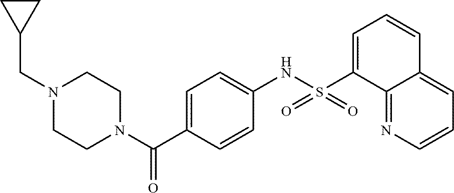 [SMILES: O=C(c1ccc(NS(=O)(=O)c2cccc3cccnc23)cc1)N1CCN(CC2CC2)CC1]

wherein the molar ratio between compound (I) and gentisic acid is 1: 1.

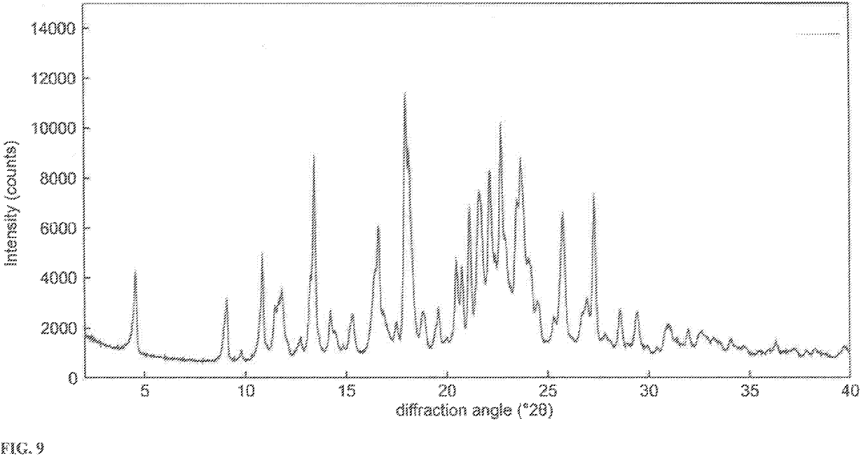

In one aspect, the gentisate salt of compound (I) is a crystalline form. In one specific embodiment, the crystalline form is crystalline Form E characterized by x-ray powder diffraction peaks at 2Θ angles (± 0.2°) 18.2°, 21.6°, 22.1°, and 22.7°. In another specific embodiment, crystalline Form E is characterized by x-ray powder diffraction peaks at 2Θ angles (± 0.2°) 18.2°, 21.6°, 22.1°, and 22.7°; and at least one, at least two, or at least three additional x-ray powder diffraction peaks at 2Θ angles (± 0.2°) selected from 13.5°, 16.5°, 18.0°, and 23.7°. In yet another specific embodiment, crystalline Form E is characterized by x-ray powder diffraction peaks at 2Θ angles (± 0.2°) 13.5°, 16.5°, 18.0°, 18.2°, 21.6°, 22.1°, 22.7°, and 23.7°. In yet another specific embodiment, crystalline Form E is characterized by x-ray powder diffraction peaks at 2Θ angles (± 0.2°) 11.8°, 13.5°, 16.5°, 18.0°, 18.2°, 21.6°, 22.1°, 22.7°, 23.7°, 24.1°, 25.8°, and 27.3°. In yet another specific embodiment, crystalline Form E is characterized by an X-ray powder diffraction pattern substantially similar to FIG. 9.

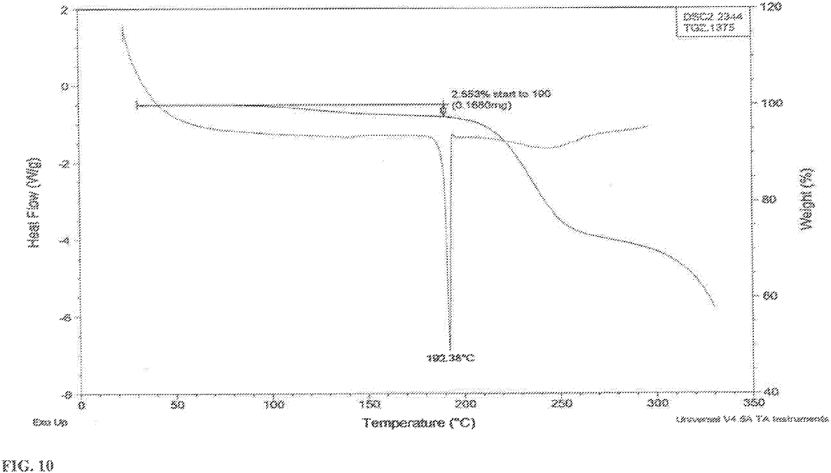

In one alternative specific embodiment, crystalline Form E is characterized by a Differential Scanning Calorimetry (DSC) with a sharp endotherm at 192.4 (onset temperature), or Thermogravimetric analysis (TGA) of a 2.6% weight loss between 20 and 190° C., or both, wherein the crystalline Form E may also comprise XRPD peaks at 2Θ angles selected from any of those described above. Alternatively, crystalline Form E is characterized by a Differential Scanning Calorimetry (DSC) or Thermogravimetric analysis (TGA) substantially similar to FIG. 10, wherein the crystalline Form E may also comprise XRPD peaks at 2Θ angles selected from any of those described above.

In another alternative, crystalline Form E as described in the above specific embodiments is at least 60% a single crystalline form, at least 70% a single crystalline form, at least 80% a single crystalline form, at least 90% a single crystalline form, at least 95% a single crystalline form, or at least 99% a single crystalline form by weight.

In yet another alternative, crystalline Form E as described in the above specific embodiments has a chemical purity of at least 60%, at least 70%, at least 80%, at least 90%, at least 95%, or at least 99% by weight.

Also provided herein is a hydrochloride salt of compound (I) represented by the following structural formula:

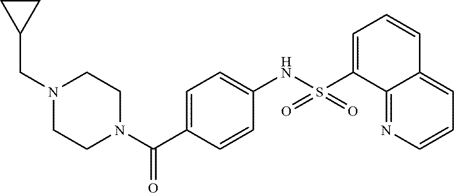 [SMILES: O=C(c1ccc(NS(=O)(=O)c2cccc3cccnc23)cc1)N1CCN(CC2CC2)CC1]

wherein the molar ratio between compound (I) and hydrochloric acid is 1: 1.

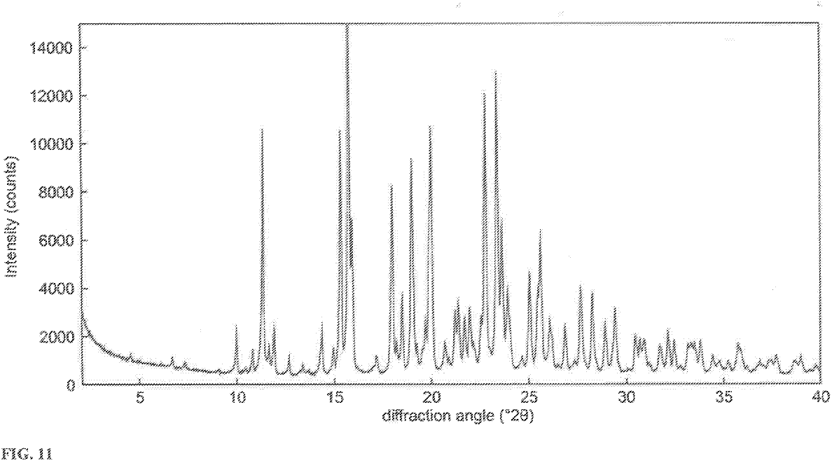

In one aspect, the hydrochloride salt of compound (I) is a crystalline form. In one specific aspect, the hydrochloride salt of compound (I) is a solvate. Further specified, the hydrochloride salt of compound (I) is a hydrate. In one embodiment, the crystalline form is crystalline Form F characterized by x-ray powder diffraction peaks at 2Θ angles (± 0.2°) 11.3°, 15.3°, 15.8°, and 23.4°. In another embodiment, crystalline Form F is characterized by x-ray powder diffraction peaks at 2Θ angles (± 0.2°) 11.3°, 15.3°, 15.8°, and 23.4°; and at least one, at least two, or at least three additional x-ray powder diffraction peaks at 2Θ angles (± 0.2°) selected from 18.0°, 19.0°, 19.9°, and 22.8°. In yet another embodiment, crystalline Form F is characterized by x-ray powder diffraction peaks at 2Θ angles (± 0.2°) 11.3°, 15.3°, 15.8°, 18.0°, 19.0°, 19.9°, 22.8°, and 23.4°. In yet another embodiment, crystalline Form F is characterized by x-ray powder diffraction peaks at 2Θ angles (± 0.2°) 11.3°, 15.3°, 15.8°, 15.9°, 18.0°, 19.0°, 19.9°, 20.0°, 22.8°, 23.4°, 23.6°, 25.6°, and 27.7°. In yet another embodiment, crystalline Form F is characterized by an X-ray powder diffraction pattern substantially similar to FIG. 11.

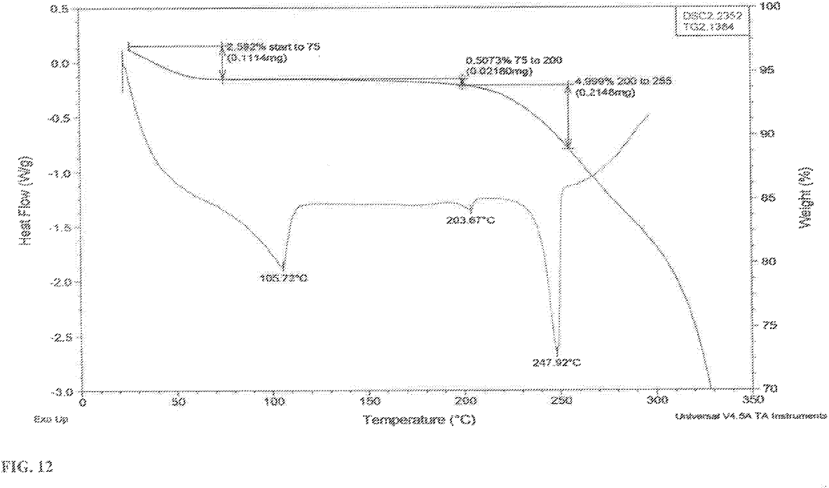

In one alternative embodiment, crystalline Form F is characterized by a Differential Scanning Calorimetry (DSC) with three endotherms at 105.7, 203.7 and 247.9° C. (onset temperatures), or Thermogravimetric analysis (TGA) of a 2.6% weight loss between 20 and 75° C. as well as a 0.5% weight loss between 75 and 200° C., or both, wherein the crystalline Form F may also comprise XRPD peaks at 2Θ angles selected from any of those described above. Alternatively, crystalline Form F is characterized by a Differential Scanning Calorimetry (DSC) or Thermogravimetric analysis (TGA) substantially similar to FIG. 12, wherein the crystalline Form F may also comprise XRPD peaks at 2Θ angles selected from any of those described above.

In another alternative, crystalline Form F as described in the above embodiments is at least 60% a single crystalline form, at least 70% a single crystalline form, at least 80% a single crystalline form, at least 90% a single crystalline form, at least 95% a single crystalline form, or at least 99% a single crystalline form by weight.

In yet another alternative, crystalline Form F as described in the above embodiments has a chemical purity of at least 60%, at least 70%, at least 80%, at least 90%, at least 95%, or at least 99% by weight.

Also provided herein is a hydrochloride salt of compound (I) represented by the following structural formula:

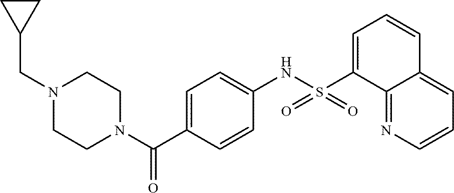 [SMILES: O=C(c1ccc(NS(=O)(=O)c2cccc3cccnc23)cc1)N1CCN(CC2CC2)CC1]

wherein the molar ratio between compound (I) and hydrochloric acid is 1: 1.

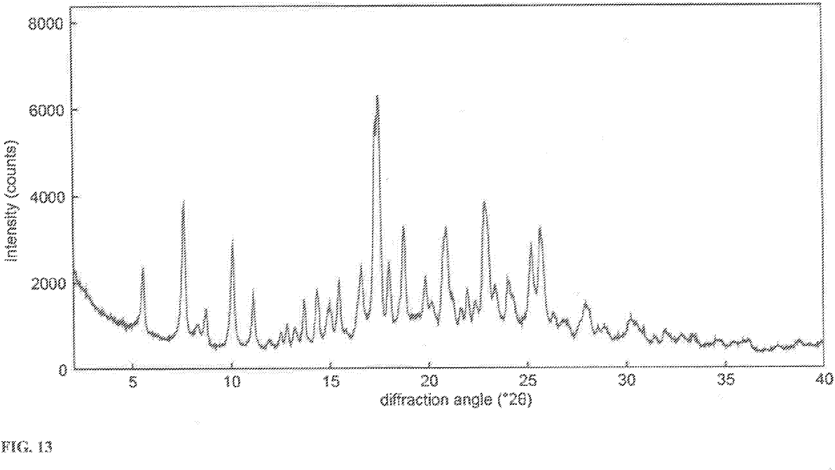

In one aspect, the hydrochloride salt of compound (I) is a crystalline form. In one embodiment of the aspect, the hydrochloride salt of compound (I) is anhydrous. In one specific embodiment, the crystalline form is crystalline Form G characterized by x-ray powder diffraction peaks at 2Θ angles (± 0.2°) 7.7°, 17.5°, 22.9°, and 25.7°. In another specific embodiment, crystalline Form G is characterized by x-ray powder diffraction peaks at 2Θ angles (± 0.2°) 7.7°, 17.5°, 22.9°, and 25.7°; and at least one, at least two, or at least three additional x-ray powder diffraction peaks at 2Θ angles (± 0.2°) selected from 10.1°, 17.3°, 20.9°, and 25.2°. In yet another specific embodiment, crystalline Form G is characterized by x-ray powder diffraction peaks at 2Θ angles (± 0.2°) 7.7°, 10.1°, 17.3°, 17.5°, 20.9°, 22.9°, 25.2°, and 25.7°. In yet another specific embodiment, crystalline Form G is characterized by x-ray powder diffraction peaks at 2Θ angles (± 0.2°) 5.6°, 7.7°, 10.1°, 16.6°, 17.3°, 17.5°, 18.8°, 20.9°, 22.9°, 25.2°, and 25.7°. In yet another specific embodiment, crystalline Form G is characterized by an X-ray powder diffraction pattern substantially similar to FIG. 13.

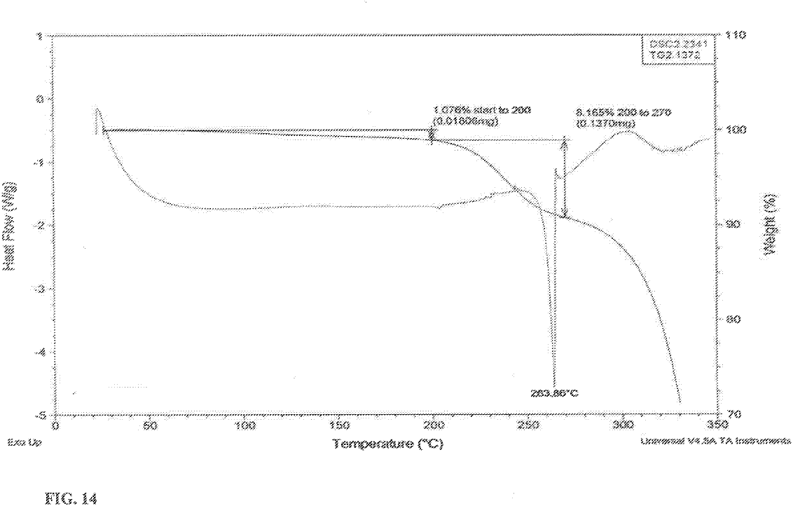

In one alternative specific embodiment, crystalline Form G is characterized by a Differential Scanning Calorimetry (DSC) with a sharp endotherm at 263.9° C. (onset temperature), or Thermogravimetric analysis (TGA) of a 1.1% weight loss between 20 and 200° C., or both, wherein the crystalline Form G may also comprise XRPD peaks at 2Θ angles selected from any of those described above. Alternatively, crystalline Form G is characterized by a Differential Scanning Calorimetry (DSC) or Thermogravimetric analysis (TGA) substantially similar to FIG. 14, wherein the crystalline Form G may also comprise XRPD peaks at 2Θ angles selected from any of those described above.

In another alternative, crystalline Form G as described in the above specific embodiments is at least 60% a single crystalline form, at least 70% a single crystalline form, at least 80% a single crystalline form, at least 90% a single crystalline form, at least 95% a single crystalline form, or at least 99% a single crystalline form by weight.

In yet another alternative, crystalline Form G as described in the above specific embodiments has a chemical purity of at least 60%, at least 70%, at least 80%, at least 90%, at least 95%, or at least 99% by weight.

Also provided herein is a maleate salt of compound (I) represented by the following structural formula:

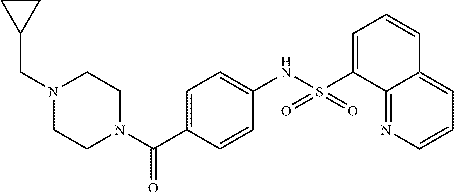 [SMILES: O=C(c1ccc(NS(=O)(=O)c2cccc3cccnc23)cc1)N1CCN(CC2CC2)CC1]

wherein the molar ratio between compound (I) and maleic acid is 1:1.

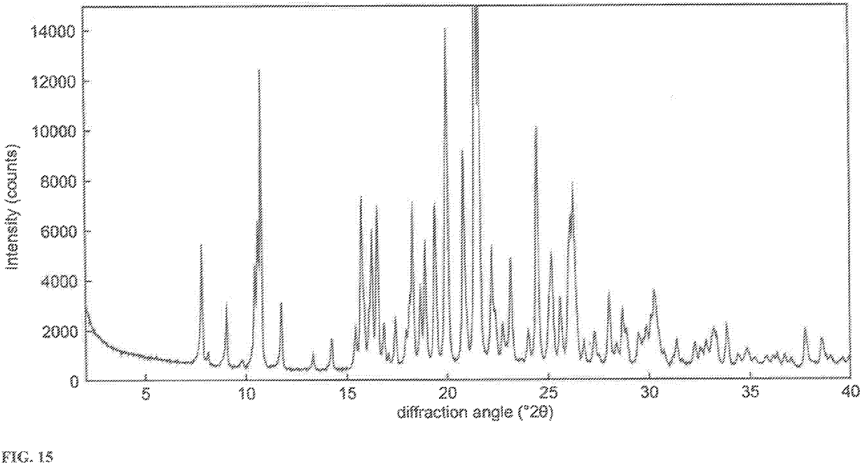

In one aspect, the maleate salt of compound (I) is a crystalline form. In one embodiment of the aspect, the maleate salt of compound (I) is anhydrous. In one specific embodiment, the crystalline form is crystalline Form H characterized by x-ray powder diffraction peaks at 2Θ angles (± 0.2°) 21.4°, 21.6°, 24.5°, and 26.2°. In another specific embodiment, crystalline Form H is characterized by x-ray powder diffraction peaks at 2Θ angles (± 0.2°) 21.4°, 21.6°, 24.5°, and 26.2°; and at least one, at least two, or at least three additional x-ray powder diffraction peaks at 2Θ angles (± 0.2°) selected from 10.8°, 19.9°, 20.0°, and 20.8°. In yet another specific embodiment, crystalline Form H is characterized by x-ray powder diffraction peaks at 2Θ angles (± 0.2°) 10.8°, 19.9°, 20.0°, 20.8°, 21.4°, 21.6°, 24.5°, and 26.2°. In yet another specific embodiment, crystalline Form H is characterized by x-ray powder diffraction peaks at 2Θ angles (± 0.2°) 10.8°, 15.8°, 16.5°, 18.3°, 19.4°, 19.9°, 20.0°, 20.8°, 21.4°, 21.6°, 24.5°, and 26.2°. In yet another specific embodiment, crystalline Form H is characterized by an X-ray powder diffraction pattern substantially similar to FIG. 15.

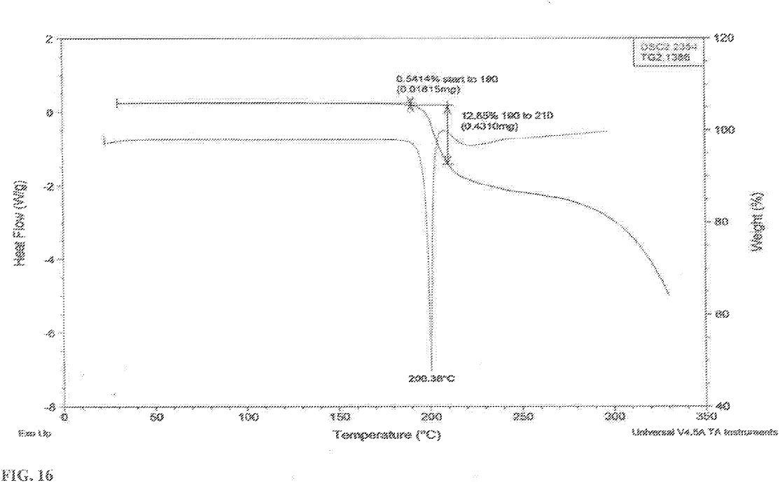

In one alternative specific embodiment, crystalline Form H is characterized by a Differential Scanning Calorimetry (DSC) with a sharp endotherm at 200.4° C. (onset temperature), or Thermogravimetric analysis (TGA) of a 0.5% weight loss between 20 and 190° C., or both, wherein the crystalline Form H may also comprise XRPD peaks at 2Θ angles selected from any of those described above. Alternatively, crystalline Form H is characterized by a Differential Scanning Calorimetry (DSC) or Thermogravimetric analysis (TGA) substantially similar to FIG. 16, wherein the crystalline Form H may also comprise XRPD peaks at 2Θ angles selected from any of those described above.

In another alternative, crystalline Form H as described in the above specific embodiments is at least 60% a single crystalline form, at least 70% a single crystalline form, at least 80% a single crystalline form, at least 90% a single crystalline form, at least 95% a single crystalline form, or at least 99% a single crystalline form by weight.

In yet another alternative, crystalline Form H as described in the above specific embodiments has a chemical purity of at least 60%, at least 70%, at least 80%, at least 90%, at least 95%, or at least 99% by weight.

Also provided herein is a malonate salt of compound (I) represented by the following structural formula:

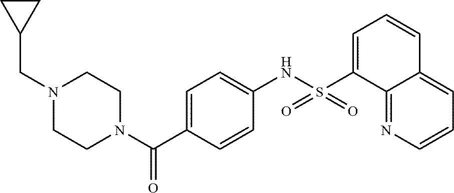 [SMILES: O=C(c1ccc(NS(=O)(=O)c2cccc3cccnc23)cc1)N1CCN(CC2CC2)CC1]

wherein the molar ratio between compound (I) and malonic acid is 1: 1.

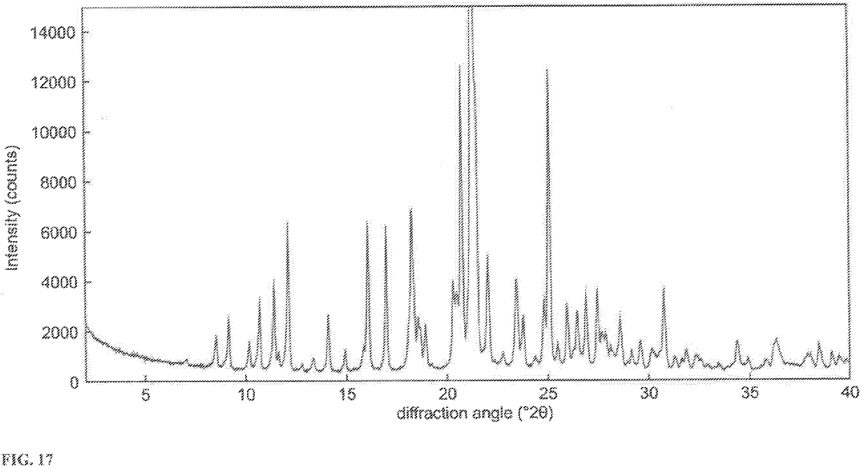

In one aspect, the malonate salt of compound (I) is a crystalline form. In one embodiment of the aspect, the malonate salt of compound (I) is anhydrous. In one specific embodiment, the crystalline form is crystalline Form I characterized by x-ray powder diffraction peaks at 2Θ angles (± 0.2°) 20.3°, 20.7°, 21.3°, and 25.1°. In another specific embodiment, crystalline Form I is characterized by x-ray powder diffraction peaks at 2Θ angles (± 0.2°) 20.3°, 20.7°, 21.3°, and 25.1°; and at least one, at least two, or at least three additional x-ray powder diffraction peaks at 2Θ angles (± 0.2°) selected from 12.1°, 17.0°, 18.2°, and 21.5°. In yet another specific embodiment, crystalline Form I is characterized by x-ray powder diffraction peaks at 2Θ angles (± 0.2°) 12.1°, 17.0°, 18.2°, 20.3°, 20.7°, 21.3°, 21.5°, and 25.1°. In yet another specific embodiment, crystalline Form I is characterized by x-ray powder diffraction peaks at 2Θ angles (± 0.2°) 12.1°, 16.1°, 17.0°, 18.2° (doublet), 20.3°, 20.7°, 21.3°, 21.5°, 22.0°, 23.4°, and 25.1°. In yet another specific embodiment, crystalline Form I is characterized by an X-ray powder diffraction pattern substantially similar to FIG. 17.

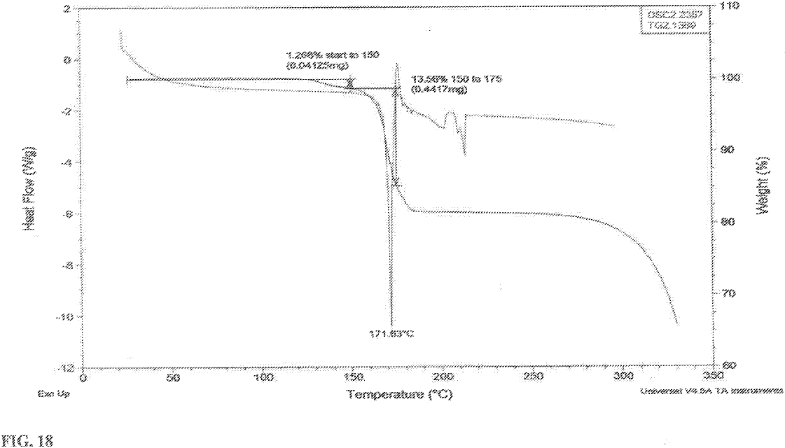

In one alternative specific embodiment, crystalline Form I is characterized by a Differential Scanning Calorimetry (DSC) with a sharp endotherm at 171.6° C. (onset temperature), or Thermogravimetric analysis (TGA) of a 1.3% weight loss between 20 and 150° C., or both, wherein the crystalline Form I may also comprise XRPD peaks at 2Θ angles selected from any of those described above. Alternatively, crystalline Form I is characterized by a Differential Scanning Calorimetry (DSC) or Thermogravimetric analysis (TGA) substantially similar to FIG. 18, wherein the crystalline Form I may also comprise XRPD peaks at 2Θ angles selected from any of those described above.

In another alternative, crystalline Form I as described in the above specific embodiments is at least 60% a single crystalline form, at least 70% a single crystalline form, at least 80% a single crystalline form, at least 90% a single crystalline form, at least 95% a single crystalline form, or at least 99% a single crystalline form by weight.

In yet another alternative, crystalline Form I as described in the above specific embodiments has a chemical purity of at least 60%, at least 70%, at least 80%, at least 90%, at least 95%, or at least 99% by weight.

Also provided herein is a phosphate salt of compound (I) represented by the following structural formula:

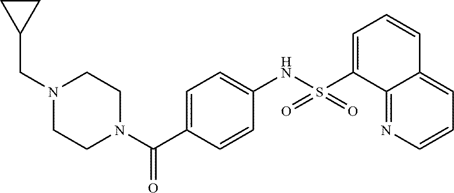 [SMILES: O=C(c1ccc(NS(=O)(=O)c2cccc3cccnc23)cc1)N1CCN(CC2CC2)CC1]

wherein the molar ratio between compound (I) and phosphoric acid is 1: 1.

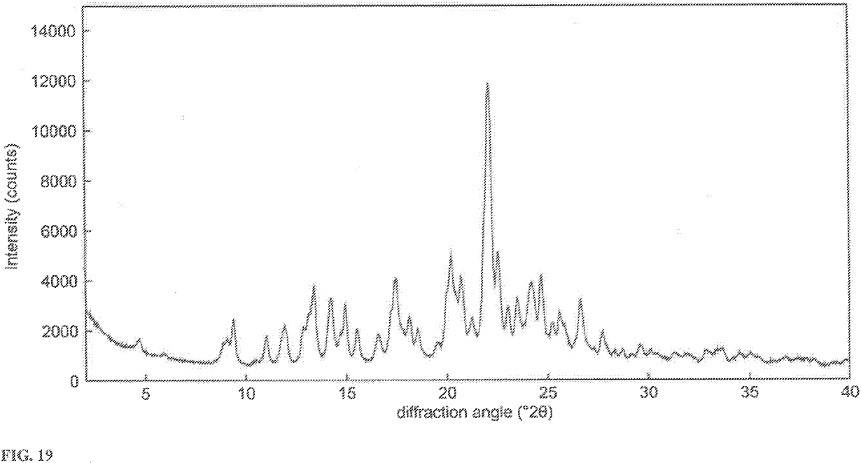

In one aspect, the phosphate salt of compound (I) is a crystalline form. In one specific aspect, the phosphate salt of compound (I) is a solvate. Further specified, the phosphate salt of compound (I) is a hydrate. In one embodiment, the crystalline form is crystalline Form J characterized by x-ray powder diffraction peaks at 2Θ angles (± 0.2°) 17.4°, 20.0°, 21.9°, and 22.1°. In another embodiment, crystalline Form J is characterized by x-ray powder diffraction peaks at 2Θ angles (± 0.2°) 17.4°, 20.0°, 21.9°, and 22.1°; and at least one, at least two, or at least three additional x-ray powder diffraction peaks at 2Θ angles (± 0.2°) selected from 12.8°, 14.2°, 22.5°, and 24.2°. In yet another embodiment, crystalline Form J is characterized by x-ray powder diffraction peaks at 2Θ angles (± 0.2°) 12.8°, 14.2°, 17.4°, 20.0°, 21.9°, 22.1°, 22.5°, and 24.2°. In yet another embodiment, crystalline Form J is characterized by x-ray powder diffraction peaks at 2Θ angles (± 0.2°) 12.8°, 13.4°, 14.2°, 15.0°, 17.4°, 20.0°, 20.7°, 21.9°, 22.1°, 22.5°, 24.2°, and 24.7°. In yet another embodiment, crystalline Form J is characterized by an X-ray powder diffraction pattern substantially similar to FIG. 19.

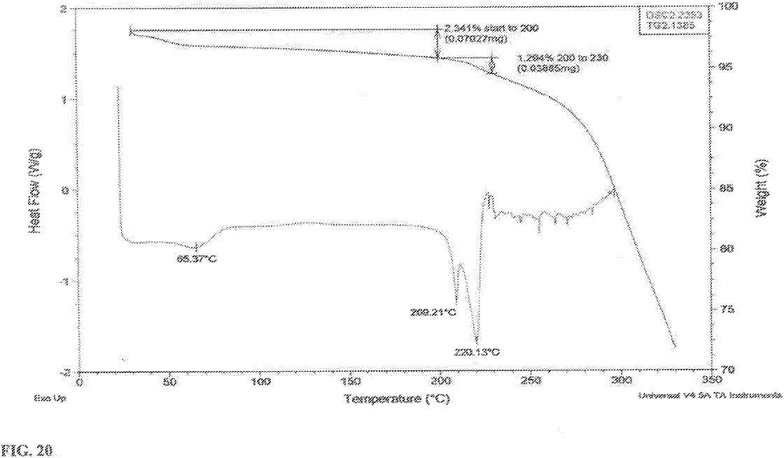

In one alternative embodiment, crystalline Form J is characterized by a Differential Scanning Calorimetry (DSC) with three endotherms at 65.4, 209.2 and 220.1° C. (onset temperatures), or Thermogravimetric analysis (TGA) of a 2.3% weight loss between 20 and 200° C., or both, wherein the crystalline Form J may also comprise XRPD peaks at 2Θ angles selected from any of those described above. Alternatively, crystalline Form J is characterized by a Differential Scanning Calorimetry (DSC) or Thermogravimetric analysis (TGA) substantially similar to FIG. 20, wherein the crystalline Form J may also comprise XRPD peaks at 2Θ angles selected from any of those described above.

In another alternative, crystalline Form J as described in the above embodiments is at least 60% a single crystalline form, at least 70% a single crystalline form, at least 80% a single crystalline form, at least 90% a single crystalline form, at least 95% a single crystalline form, or at least 99% a single crystalline form by weight.

In yet another alternative, crystalline Form J as described in the above embodiments has a chemical purity of at least 60%, at least 70%, at least 80%, at least 90%, at least 95%, or at least 99% by weight.

Also provided herein is a phosphate salt of compound (I) represented by the following structural formula:

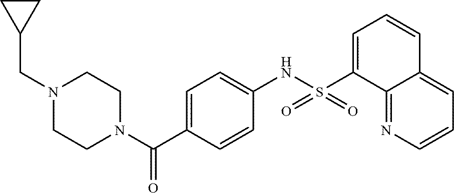 [SMILES: O=C(c1ccc(NS(=O)(=O)c2cccc3cccnc23)cc1)N1CCN(CC2CC2)CC1]

wherein the molar ratio between compound (I) and phosphoric acid is 1:1.

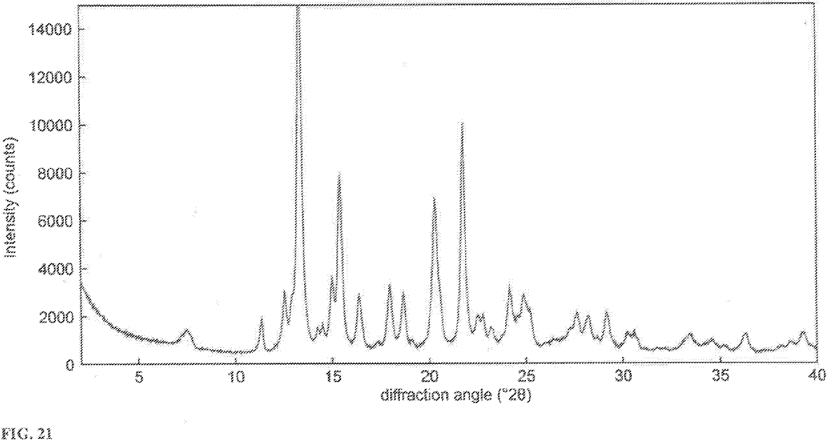

In one aspect, the phosphate salt of compound (I) is a crystalline form. In one embodiment of the aspect, the phosphate salt of compound (I) is anhydrous. In one specific embodiment, the crystalline form is crystalline Form K characterized by x-ray powder diffraction peaks at 2Θ angles (± 0.2°) 13.4°, 15.4°, 20.3°, and 21.8°. In another specific embodiment, crystalline Form K is characterized by x-ray powder diffraction peaks at 2Θ angles (± 0.2°) 13.4°, 15.4°, 20.3°, and 21.8°; and at least one, at least two, or at least three additional x-ray powder diffraction peaks at 2Θ angles (± 0.2°) selected from 15.0°, 17.9°, 24.9°, and 27.6°. In yet another specific embodiment, crystalline Form K is characterized by x-ray powder diffraction peaks at 2Θ angles (± 0.2°) 13.4°, 15.0°, 15.4°, 17.9°, 20.3°, 21.8°, 24.9°, and 27.6°. In yet another specific embodiment, crystalline Form K is characterized by x-ray powder diffraction peaks at 2Θ angles (± 0.2°) 12.6°, 12.9°, 13.4°, 15.0°, 15.4°, 16.4°, 17.9°, 18.7°, 20.3°, 21.8°, 24.9°, and 27.6°. In yet another specific embodiment, crystalline Form K is characterized by an X-ray powder diffraction pattern substantially similar to FIG. 21.

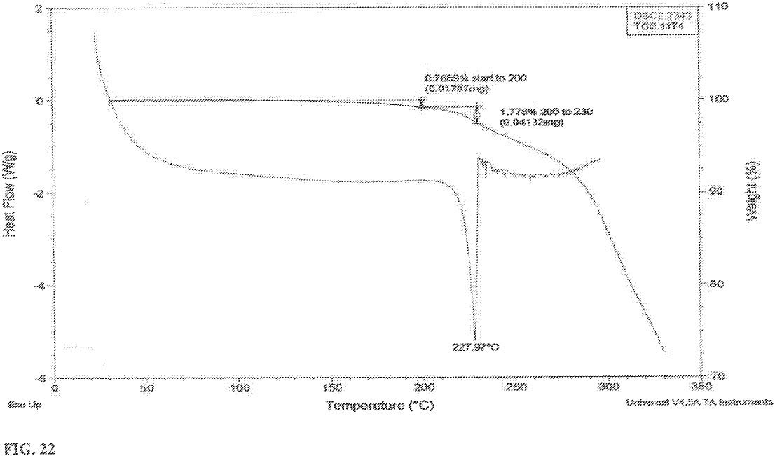

In one alternative specific embodiment, crystalline Form K is characterized by a Differential Scanning Calorimetry (DSC) with a sharp endotherm at 228.0° C. (onset temperature), or Thermogravimetric analysis (TGA) of a 0.8% weight loss between 20 and 200° C., or both, wherein the crystalline Form K may also comprise XRPD peaks at 2Θ angles selected from any of those described above. Alternatively, crystalline Form K is characterized by a Differential Scanning Calorimetry (DSC) or Thermogravimetric analysis (TGA) substantially similar to FIG. 22, wherein the crystalline Form K may also comprise XRPD peaks at 2Θ angles selected from any of those described above.

In another alternative, crystalline Form K as described in the above specific embodiments is at least 60% a single crystalline form, at least 70% a single crystalline form, at least 80% a single crystalline form, at least 90% a single crystalline form, at least 95% a single crystalline form, or at least 99% a single crystalline form by weight.

In yet another alternative, crystalline Form K as described in the above specific embodiments has a chemical purity of at least 60%, at least 70%, at least 80%, at least 90%, at least 95%, or at least 99% by weight.

Also provided herein is a tartrate salt of compound (I) represented by the following structural formula:

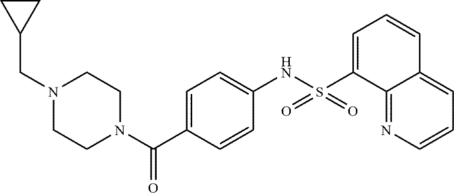 [SMILES: O=C(c1ccc(NS(=O)(=O)c2cccc3cccnc23)cc1)N1CCN(CC2CC2)CC1]

wherein the molar ratio between compound (I) and tartaric acid is 1: 1.

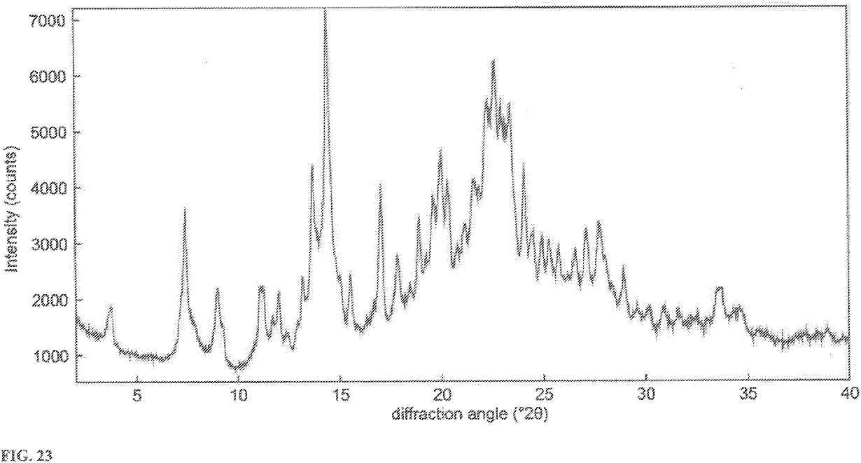

In one aspect, the tartrate salt of compound (I) is a crystalline form. In one specific aspect, the tartrate salt of compound (I) is a solvate. Further specified, the phosphate salt of compound (I) is a hydrate. In one embodiment, the crystalline form is crystalline Form L characterized by x-ray powder diffraction peaks at 2Θ angles (± 0.2°) 7.4°, 13.7°, 14.4°, and 22.7°. In another embodiment, crystalline Form L is characterized by x-ray powder diffraction peaks at 2Θ angles (± 0.2°) 7.4°, 13.7°, 14.4°, and 22.7°; and at least one, at least two, or at least three additional x-ray powder diffraction peaks at 2Θ angles (± 0.2°) selected from 14.8°, 22.9°, 23.4°, and 27.7°. In yet another embodiment, crystalline Form L is characterized by x-ray powder diffraction peaks at 2Θ angles (± 0.2°) 7.4°, 13.7°, 14.4°, 14.8°, 22.7°, 22.9°, 23.4°, and 27.7°. In yet another embodiment, crystalline Form L is characterized by x-ray powder diffraction peaks at 2Θ angles (± 0.2°) 7.4°, 13.2°, 13.7°, 14.4°, 14.8°, 17.0°, 20.0°, 21.5°, 22.2°, 22.7°, 22.9°, 23.4°, and 27.7°. In yet another embodiment, crystalline Form L is characterized by an X-ray powder diffraction pattern substantially similar to FIG. 23.

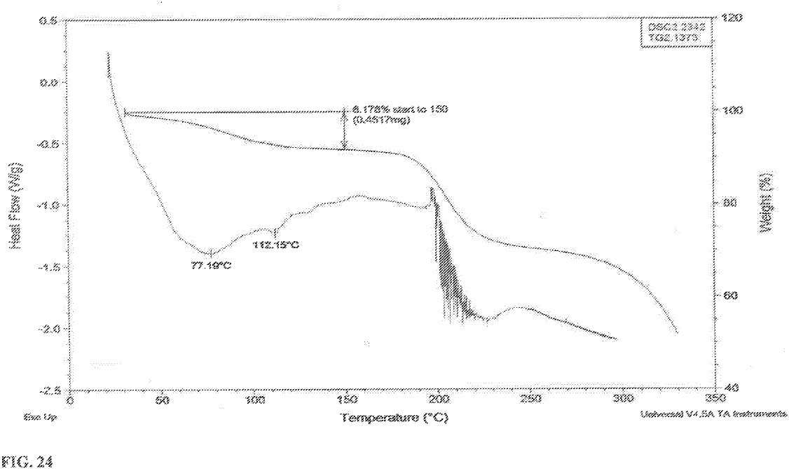

In one alternative embodiment, crystalline Form L is characterized by a Differential Scanning Calorimetry (DSC) with two endotherms at 77.2 and 112.2° C. (onset temperatures), or Thermogravimetric analysis (TGA) of a 8.2% weight loss between 20 and 150° C., or both, wherein the crystalline Form L may also comprise XRPD peaks at 2Θ angles selected from any of those described above. Alternatively, crystalline Form L is characterized by a Differential Scanning Calorimetry (DSC) or Thermogravimetric analysis (TGA) substantially similar to FIG. 24, wherein the crystalline Form L may also comprise XRPD peaks at 2Θ angles selected from any of those described above.

In another alternative, crystalline Form L as described in the above embodiments is at least 60% a single crystalline form, at least 70% a single crystalline form, at least 80% a single crystalline form, at least 90% a single crystalline form, at least 95% a single crystalline form, or at least 99% a single crystalline form by weight.

In yet another alternative, crystalline Form L as described in the above embodiments has a chemical purity of at least 60%, at least 70%, at least 80%, at least 90%, at least 95%, or at least 99% by weight.

Also provided herein is a tosylate salt of compound (I) represented by the following structural formula:

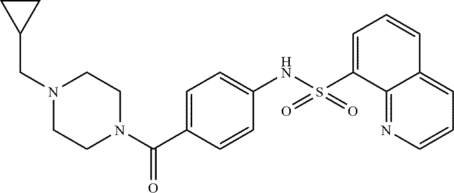 [SMILES: O=C(c1ccc(NS(=O)(=O)c2cccc3cccnc23)cc1)N1CCN(CC2CC2)CC1]

wherein the molar ratio between compound (I) and tosylic acid is 1:1.

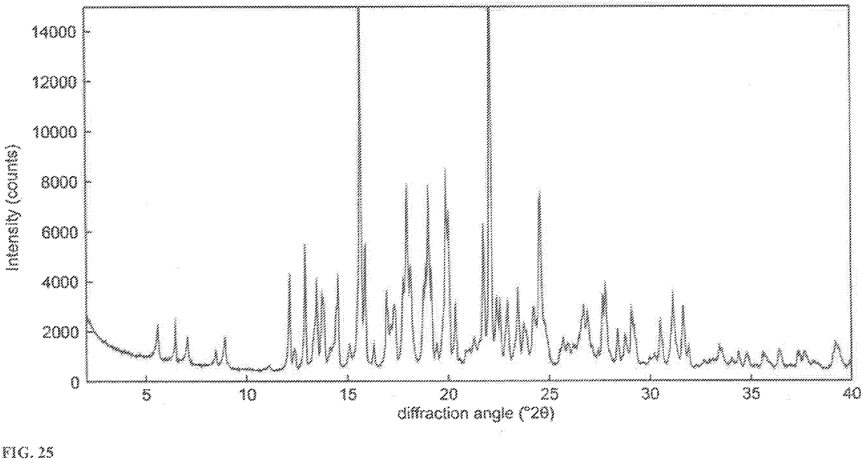

In one aspect, the phosphate salt of compound (I) is a crystalline form. In one embodiment of the aspect, the phosphate salt of compound (I) is anhydrous. In one specific embodiment, the crystalline form is crystalline Form M characterized by x-ray powder diffraction peaks at 2Θ angles (± 0.2°) 15.7°, 17.8°, 22.1°, and 24.5°. In another specific embodiment, crystalline Form M is characterized by x-ray powder diffraction peaks at 2Θ angles (± 0.2°) 15.7°, 17.8°, 22.1°, and 24.5°; and at least one, at least two, or at least three additional x-ray powder diffraction peaks at 2Θ angles (± 0.2°) selected from 12.9°, 15.9°, 18.8°, and 21.8°. In yet another specific embodiment, crystalline Form M is characterized by x-ray powder diffraction peaks at 2Θ angles (± 0.2°) 12.9°, 15.7°, 15.9°, 17.8°, 18.8°, 21.8°, 22.1°, and 24.5°. In yet another specific embodiment, crystalline Form M is characterized by x-ray powder diffraction peaks at 2Θ angles (± 0.2°) 12.9°, 13.5°, 15.7°, 15.9°, 17.8°, 18.8°, 19.0°, 19.8°, 20.0°, 21.8°, 22.1°, and 24.5°. In yet another specific embodiment, crystalline Form M is characterized by an X-ray powder diffraction pattern substantially similar to FIG. 25.

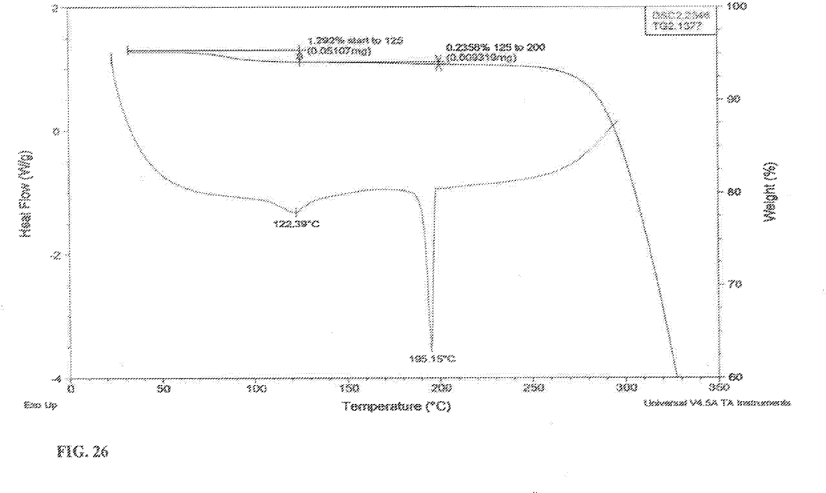

In one alternative specific embodiment, crystalline Form M is characterized by a Differential Scanning Calorimetry (DSC) with two endotherms at 122.4 and 195.2° C. (onset temperatures), or Thermogravimetric analysis (TGA) of a 1.3% weight loss between 20 and 125° C. as well as a 0.2% weight loss between 125 and 200° C., or both, wherein the crystalline Form M may also comprise XRPD peaks at 2Θ angles selected from any of those described above. Alternatively, crystalline Form M is characterized by a Differential Scanning Calorimetry (DSC) or Thermogravimetric analysis (TGA) substantially similar to FIG. 26, wherein the crystalline Form M may also comprise XRPD peaks at 2Θ angles selected from any of those described above.

In another alternative, crystalline Form M as described in the above specific embodiments is at least 60% a single crystalline form, at least 70% a single crystalline form, at least 80% a single crystalline form, at least 90% a single crystalline form, at least 95% a single crystalline form, or at least 99% a single crystalline form by weight.

In yet another alternative, crystalline Form M as described in the above specific embodiments has a chemical purity of at least 60%, at least 70%, at least 80%, at least 90%, at least 95%, or at least 99% by weight.

### Compositions and Administration

Provided herein are pharmaceutical compositions comprising one or more of the disclosed crystalline forms (e.g. crystalline Form A), together with a pharmaceutically acceptable carrier. The amount of crystalline form in a provided composition is such that is effective to measurably modulate PKR in a subject.

Pharmaceutical compositions described herein can be prepared by any method known in the art of pharmacology. In general, such preparatory methods include the steps of bringing one or more of the disclosed crystalline forms (e.g. crystalline Form A) into association with a carrier and/or one or more other accessory ingredients, and then, if necessary and/or desirable, shaping and/or packaging the product into a desired single- or multi-dose unit.

Pharmaceutically acceptable carriers used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Carriers such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite (aluminum silicate) and Veegum (magnesium aluminum silicate)), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80), polyoxyethylene esters (e.g. polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g. Cremophor™), polyoxyethylene ethers, (e.g. polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F-68, Poloxamer-188, cetrimonium bromide, cetylpyridinium chloride, benzalkonium chloride, docusate sodium, and/or mixtures thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer’s solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, sodium stearyl fumarate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Compositions described herein may be administered orally, parenterally, by inhalation spray, topically, rectally, nasally, buccally, transmucosally, or in an ophthalmic preparation. The term “parenteral” as used herein includes subcutaneous, intravenous, intramuscular, intra-articular, intra-synovial, intrasternal, intrathecal, intrahepatic, intralesional and intracranial injection or infusion techniques. In one aspect, the pharmaceutical compositions provided herewith are orally administered in an orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The amount of provided crystalline form that may be combined with carrier materials to produce a composition in a single dosage form will vary depending upon the subject to be treated and the particular mode of administration. For example, a specific dosage and treatment regimen for any particular subject will depend upon a variety of factors, including age, body weight, general health, sex, diet, time of administration, rate of excretion, drug combination, the judgment of the treating physician, and the severity of the particular disease being treated. The amount of a provided crystalline form in the composition will also depend upon the particular form (e.g., Form A, B, C, D, E, F, G, H, I, J, K, L, or M) in the composition. In one aspect, a provided composition may be formulated such that a dosage equivalent to about 0.001 to about 100 mg/kg body weight/day of Compound (I) (e.g., about 0.5 to about 100 mg/kg of Compound (I)) can be administered to a subject receiving these compositions. Alternatively, dosages equivalent to 1 mg/kg and 1000 mg/kg of Compound (I) every 4 to 120 hours is also acceptable. As used herein, the dose refers to the amount of Compound (I) in the particular crystalline form. The amount of the particular crystalline form will be calculated based on the equivalence to the free-base form of Compound (I).

In one aspect, crystalline Form A, B, C, D, E, F, G, H, I, J, K, L, or M is formulated for administration at a dose of equivalent to about 2 mg to about 3000 mg of Compound (I). In certain embodiments, the dose is oral dose. In certain embodiments, crystalline Form A, B, C, D, E, F, G, H, I, J, K, L, or M is formulated equivalent to about 2 mg to about 3000 mg of Compound (I). In certain embodiments, a disclosed crystalline (e.g. crystalline Form A) form is formulated equivalent to about 5 mg to about 350 mg of Compound (I). In certain embodiments, crystalline Form A, B, C, D, E, F, G, H, I, J, K, L, or M is formulated equivalent to about 5 mg to about 200 mg of Compound (I). In certain embodiments, crystalline Form A, B, C, D, E, F, G, H, I, J, K, L, or M is formulated equivalent to about 5 mg to about 100 mg of Compound (I). In certain embodiments, crystalline Form A, B, C, D, E, F, G, H, I, J, K, L, or M is formulated equivalent to about 5 mg of Compound (I). In certain embodiments, crystalline Form A, B, C, D, E, F, G, H, I, J, K, L, or M is formulated equivalent to about 10 mg of Compound (I). In certain embodiments, crystalline Form A, B, C, D, E, F, G, H, I, J, K, L, or M is formulated equivalent to about 15 mg of Compound (I). In certain embodiments, crystalline Form A, B, C, D, E, F, G, H, I, J, K, L, or M is formulated equivalent to about 20 mg of Compound (I). In certain 25 mg. In certain embodiments, crystalline Form A, B, C, D, E, F, G, H, I, J, K, L, or M is formulated equivalent to about 30 mg of Compound (I). In certain embodiments, crystalline Form A, B, C, D, E, F, G, H, I, J, K, L, or M is formulated equivalent to about 40 mg of Compound (I). In certain embodiments, crystalline Form A, B, C, D, E, F, G, H, I, J, K, L, or M is formulated equivalent to about 45 mg of Compound (I). In certain embodiments, crystalline Form A, B, C, D, E, F, G, H, I, J, K, L, or M is formulated equivalent to about 50 mg of Compound (I). In certain embodiments, crystalline Form A, B, C, D, E, F, G, H, I, J, K, L, or M is formulated equivalent to about 60 mg of Compound (I). In certain embodiments, crystalline Form A, B, C, D, E, F, G, H, I, J, K, L, or M is formulated equivalent to about 70 mg of Compound (I). In certain embodiments, crystalline Form A, B, C, D, E, F, G, H, I, J, K, L, or M is formulated equivalent to about 80 mg of Compound (I). In certain embodiments, crystalline Form A, B, C, D, E, F, G, H, I, J, K, L, or M is formulated equivalent to about 90 mg of Compound (I). In certain embodiments, crystalline Form A, B, C, D, E, F, G, H, I, J, K, L, or M is formulated equivalent to about 100 mg of Compound (I). In certain embodiments, crystalline Form A, B, C, D, E, F, G, H, I, J, K, L, or M is formulated equivalent to about 110 mg of Compound (I). In certain embodiments, crystalline Form A, B, C, D, E, F, G, H, I, J, K, L, or M is formulated equivalent to about 120 mg of Compound (I).

In certain embodiments, crystalline Form A, B, C, D, E, F, G, H, I, J, K, L, or M is formulated for administration at a dose equivalent to about 2 mg to about 3000 mg of Compound (I) per day. In certain embodiments, crystalline Form A, B, C, D, E, F, G, H, I, J, K, L, or M is formulated for administration at a dose equivalent to about 5 mg to about 500 mg of Compound (I) per day. In certain embodiments, crystalline Form A, B, C, D, E, F, G, H, I, J, K, L, or M is formulated for administration at a dose equivalent to about 5 mg to about 200 mg of Compound (I) per day. In certain embodiments, crystalline Form A, B, C, D, E, F, G, H, I, J, K, L, or M is formulated for administration at a dose equivalent to about 5 mg of Compound (I) per day. In certain embodiments, crystalline Form A, B, C, D, E, F, G, H, I, J, K, L, or M is formulated for administration at a dose equivalent to about 5 mg to about 10 mg of Compound (I) per day. In certain embodiments, crystalline Form A, B, C, D, E, F, G, H, I, J, K, L, or M is formulated for administration at a dose of about 15 mg equivalent to Compound (I) per day. In certain embodiments, crystalline Form A, B, C, D, E, F, G, H, I, J, K, L, or M is formulated for administration at a dose equivalent to about 20 mg of Compound (I) per day. In certain embodiments, crystalline Form A, B, C, D, E, F, G, H, I, J, K, L, or M is formulated for administration at a dose equivalent to about 25 mg of Compound (I) per day. In certain embodiments, crystalline Form A, B, C, D, E, F, G, H, I, J, K, L, or M is formulated for administration at a dose equivalent to about 30 mg of Compound (I) per day. In certain embodiments, crystalline Form A, B, C, D, E, F, G, H, I, J, K, L, or M is formulated for administration at a dose equivalent to about 35 mg of Compound (I) per day. In certain embodiments, crystalline Form A, B, C, D, E, F, G, H, I, J, K, L, or M is formulated for administration at a dose equivalent to about 40 mg of Compound (I) per day. In certain embodiments, crystalline Form A, B, C, D, E, F, G, H, I, J, K, L, or M is formulated for administration at a dose equivalent to about 45 mg of Compound (I) per day. In certain embodiments, crystalline Form A, B, C, D, E, F, G, H, I, J, K, L, or M is formulated for administration at a dose equivalent to about 50 mg of Compound (I) per day. In certain embodiments, crystalline Form A, B, C, D, E, F, G, H, I, J, K, L, or M is formulated for administration at a dose equivalent to about 60 mg of Compound (I) per day. In certain embodiments, crystalline Form A, B, C, D, E, F, G, H, I, J, K, L, or M is formulated for administration at a dose equivalent to about 70 mg of Compound (I) per day. In certain embodiments, crystalline Form A, B, C, D, E, F, G, H, I, J, K, L, or M is formulated for administration at a dose equivalent to about 80 mg of Compound (I) per day. In certain embodiments, crystalline Form A, B, C, D, E, F, G, H, I, J, K, L, or M is formulated for administration at a dose equivalent to about 90 mg of Compound (I) per day. In certain embodiments, crystalline Form A, B, C, D, E, F, G, H, I, J, K, L, or M is formulated for administration at a dose equivalent to about 100 mg of Compound (I) per day. In certain embodiments, crystalline Form A, B, C, D, E, F, G, H, I, J, K, L, or M is formulated for administration at a dose equivalent to about 110 mg of Compound (I) per day. In certain embodiments, crystalline Form A, B, C, D, E, F, G, H, I, J, K, L, or M is formulated for administration at a dose equivalent to about 120 mg of Compound (I) per day. In certain embodiments, crystalline Form A, B, C, D, E, F, G, H, I, J, K, L, or M is formulated for administration at a dose equivalent to about 130 mg of Compound (I) per day. In certain embodiments, crystalline Form A, B, C, D, E, F, G, H, I, J, K, L, or M is formulated for administration at a dose equivalent to about 140 mg of Compound (I) per day. In certain embodiments, crystalline Form A, B, C, D, E, F, G, H, I, J, K, L, or M is formulated for administration at a dose equivalent to about 150 mg of Compound (I) per day. Dosing can be once, twice, or three times daily. In one aspect, e.g., crystalline Form A, B, C, D, E, F, G, H, I, J, K, L, or M is formulated for administration at a dose equivalent to about 5 mg of Compound (I) twice per day. In one aspect, e.g., crystalline Form A, B, C, D, E, F, G, H, I, J, K, L, or M is formulated for administration at a dose equivalent to about 20 mg of Compound (I) twice per day. In one aspect, e.g., crystalline Form A, B, C, D, E, F, G, H, I, J, K, L, or M is formulated for administration at a dose equivalent to about 50 mg of Compound (I) twice per day. In one aspect, e.g., crystalline Form A, B, C, D, E, F, G, H, I, J, K, L, or M is formulated for administration at a dose equivalent to about 100 mg of Compound (I) twice per day. In one aspect, e.g., crystalline Form A, B, C, D, E, F, G, H, I, J, K, L, or M is formulated for administration at a dose equivalent to about 5 mg of Compound (I) once every other day. In one aspect, e.g., crystalline Form A, B, C, D, E, F, G, H, I, J, K, L, or M is formulated for administration at a dose equivalent to about 20 mg of Compound (I) once every other day. In one aspect, e.g., crystalline Form A, B, C, D, E, F, G, H, I, J, K, L, or M is formulated for administration at a dose equivalent to about 50 mg of Compound (I) once every other day. In one aspect, e.g., crystalline Form A, B, C, D, E, F, G, H, I, J, K, L, or M is formulated for administration at a dose equivalent to about 100 mg of Compound (I) once every other day.

In one aspect, a disclosed form (crystalline Form A, B, C, D, E, F, G, H, I, J, K, L, or M) is formulated as a tablet composition together with a pharmaceutically acceptable carrier. In one aspect, the carrier is selected from one or more of microcrystalline cellulose, mannitol, Croscarmellose Sodium, and Sodium Stearyl Fumarate. In one aspect, the carrier is microcrystalline cellulose e.g., present in an amount of 50% w/w to 70% w/w (±2%), 55% w/w to 65% w/w (±2%), 58% w/w to 62% w/w (±2%), 59% w/w (±2%), 60% w/w (±2%), 61% w/w (±2%), 62% w/w (±2%), 61% w/w, or 62% w/w. In another aspect, the carrier is mannitol e.g., present in an amount of 15% w/w (±2%) to 35% w/w (±2%), 20% w/w (±2%) to 30% w/w (±2%), 22% w/w (±2%) to 26% w/w (±2%), 22% w/w (±2%), 23% w/w (±2%), 24% w/w (±2%), or 23% w/w. In another aspect, the carrier is croscarmellose sodium e.g., present in an amount of 1% w/w to 5% w/w (±2%), 2% w/w to 4% w/w (±2%), 2% w/w (±2%), 3% w/w (±2%), 4% w/w (±2%) or 3% w/w. In another aspect, the carrier is stearyl fumarate e.g., present in an amount of 1% w/w to 5% w/w (±2%), 2% w/w to 4% w/w (±2%), 1% w/w (±2%), 2% w/w (±2%), 3% w/w (±2%) or 2% w/w. In some embodiments, crystalline Form A, B, C, D, E, F, G, H, I, J, K, L, or M is present in the tablet composition in an amount equivalent to about 1 to about 200 mg of Compound (I). In some embodiments, a disclosed crystalline form (e.g. crystalline Form A) is present in the tablet composition in an amount equivalent to about 1 to about 150 mg of Compound (I). In some embodiments, a disclosed crystalline form (e.g. crystalline Form A) is present in the tablet composition in an amount equivalent to about 1 to about 100 mg of Compound (I). In some embodiments, a disclosed crystalline form (e.g. crystalline Form A) is present in the tablet composition in an amount equivalent to about 5 mg of Compound (I). In some embodiments, a disclosed crystalline form (e.g. crystalline Form A) is present in the tablet composition in an amount equivalent to about 20 mg of Compound (I). In some embodiments, a disclosed crystalline form (e.g. crystalline Form A) is present in the tablet composition in an amount equivalent to about 50 mg of Compound (I). In some embodiments, a disclosed crystalline form (e.g. crystalline Form A) is present in the tablet composition in an amount equivalent to about 75 mg of Compound (I). In some embodiments, a disclosed crystalline form (e.g. crystalline Form A) is present in a tablet composition in an amount equivalent to about 100 mg of Compound (I).

As used herein, the dose amount of crystalline Form A, B, C, D, E, F, G, H, I, J, K, L, or M is based on the equivalence to the free-base form of Compound (I). For example, “crystalline Form A present in the composition in an amount equivalent to about 1.0 mg of Compound (I)” means about 1.18 mg of crystalline Form A is present in the composition and is equivalent to about 1.0 mg of free base Compound (I).

In one aspect, the tablet composition comprises 10% w/w (±1%) of the crystalline free-base; 62% w/w (±2%) microcrystalline cellulose; 23% w/w (±2%) mannitol, 3% w/w (±2%) croscarmellose sodium, and 2% w/w (±2%) stearyl fumarate.

In one aspect, the tablet composition comprises 11.78% w/w (±1%) of crystalline Form A; 62% w/w (±2%) microcrystalline cellulose; 23% w/w (±2%) mannitol; 3% w/w (±2%) croscarmellose sodium; and 2% w/w (±2%) stearyl fumarate.

### Methods of Treatment and Uses of Compounds and Compositions

In one aspect, the crystalline forms described herein and compositions thereof are allosteric activators of PKR, and are generally useful for treating the underlying condition of PKD.

Thus, provided herein are methods of treating Pyruvate Kinase Deficiency (PKD) in a subject in need thereof, comprising administering to the subject an effective amount of crystalline Form A, B, C, D, E, F, G, H, I, J, K, L, or M, or a pharmaceutical composition thereof. Also provided is crystalline Form A, B, C, D, E, F, G, H, I, J, K, L, or M; or a pharmaceutical composition thereof for use in treating Pyruvate Kinase Deficiency (PKD) in a subject in need thereof. Further provided is the use of crystalline Form A, B, C, D, E, F, G, H, I, J, K, L, or M, or a pharmaceutical composition thereof in the manufacture of a medicament for treating Pyruvate Kinase Deficiency (PKD). Exemplified conditions related to PKD include, but are not limited to, anemias, cholecystolithiasis, gallstones, tachycardia, hemochromatosis, icteric sclera, splenomegaly, leg ulcers, jaundice, fatigue, and shortness of breath. As described herein, PKD is a deficiency of PKR. In certain embodiments, the deficiency of PKR is associated with a PKR mutation.

Pyruvate kinase deficiency (PKD) is a glycolytic enzymopathy that results in life-long hemolytic anemia. In certain embodiments, the subject having PKD is a patient having at least 2 mutant alleles in PKLR gene. In certain embodiments, the subject having PKD is a patient having at least 2 mutant alleles in PKLR gene and at least one is a missense mutation. See Canu. et.al , Blood Cells, Molecules and Diseases 2016, 57, pp. 100-109. In certain embodiments, a subject having PKD has an Hb concentration less than or equal to 10.0 g/dL. In certain embodiments, the subject having PKD is an adult not under regular transfusion (e.g. having had no more than 4 transfusion episodes in the 12-month period up to the treatment). In certain embodiments, the subject having PKD is an adult transfusion independent (e.g. having no more than 3 units of RBCs transfused in the 12-month period prior to the treatment). In certain embodiments, the subject having PKD is an adult under regular transfusion (e.g. having had at least 4 transfusion episodes (e.g., at least 6 transfusion episodes) in the 12-month period prior to the treatment). In certain embodiments, the subject having PKD has a total number of at least 5 transfusion episodes during the subject’s lifetime. In certain embodiments, the subject having PKD has a total number of at least 10 transfusion episodes during the subject’s lifetime. In certain embodiments, the subject having PKD has a total number of at least 15 transfusion episodes during the subject’s lifetime. In certain embodiments, the subject having PKD has a total number of at least 20 transfusion episodes during the subject’s lifetime. In certain embodiments, the subject having PKD has a total number of at least 25 transfusion episodes during the subject’s lifetime. In certain embodiments, the subject having PKD has a total number of at least 30 transfusion episodes during the subject’s lifetime. In certain embodiments, the subject having PKD has a total number of at least 40 transfusion episodes during the subject’s lifetime. In certain embodiments, the subject having PKD has a total number of at least 50 transfusion episodes during the subject’s lifetime. In certain embodiments, the subject having PKD has a total number of at least 60 transfusion episodes during the subject’s lifetime. In certain embodiments, the subject having PKD has a total number of at least 70 transfusion episodes during the subject’s lifetime. In certain embodiments, the subject having PKD is not homozygous for the R479H mutation or does not have 2 non-missense mutations in the PKLR gene. In certain embodiments, the subject having PKD, under regular transfusion, has hemoglobin (Hb) ≤12.0 g/dL (if male) or ≤11.0 g/dL (if female), prior to the treatment. In certain embodiments, the subject having PKD, under regular transfusion, has transfusion occurring on average less than or equal to once every three weeks. In certain embodiments, the subject having PKD has received at least 0.8 mg (e.g. at least 1.0 mg)folic acid daily (e.g. for at least 21 days) prior to the treatment. In certain embodiments, the subject with PKD under regular transfusion achieves a reduction in transfusion burden (e.g. at least 33% reduction in the number of RBC units transfused) during the 5 weeks, 10 weeks, 15 weeks, 20 weeks, or 24 weeks, 28 weeks, or 32 weeks of treatment. In certain embodiments, the subject having PKD, not under regular transfusion (having had no more than 4 transfusion episodes in the 12-month period prior to the treatment and/or no transfusion in the 3 months prior to the treatment), has hemoglobin (Hb) ≤10.0 g/dL regardless of gender prior to the treatment. In certain embodiments, the subject having PKD has undergone splenectomy.

In certain embodiments, the subject with PKD achieves a hemoglobin response of at least 1.0 g/dL increase in Hb concentration after the treatment compared to the baseline of prior to the treatment. In certain embodiments, the subject with PKD achieves a hemoglobin response of at least 1.5 g/dL increase in Hb concentration from baseline prior to the treatment. In certain embodiments, the subject with PKD achieves a hemoglobin response of at least 2.0 g/dL increase in Hb concentration from baseline prior to the treatment.

In an embodiment, the mutant PKR is selected from the group consisting of A31V, A36G, G37Q, R40W, R40Q, L73P, S80P, P82H, R86P, I90N, T93I, G95R, M107T, G111R, A115P, S120F, H121Q, S130P, S130Y, V134D, R135D, A137T, G143S, I153T, A154T, L155P, G159V, R163C, R163L, T164N, G165V, L167M, G169G, E172Q, W201R, I219T, A221Y, D221N, G222A, I224T, G232C, N253D, G263R, G263W, E266K, V269F, L272V, L272P, G275R, G275R, E277K, V280G, D281N, F287V, F287L, V288L, D293N, D293V, A295I, A295V, I310N, I314T, E315K, N316K, V320L, V320M, S330R, D331N, D331G, D331E, G332S, V335M, A336S, R337W, R337P, R337Q, D339N, D339Q, G341A, G341D, I342F, K348N, A352D, I357T, G358R, G358E, R359C, R359H, C360Y, N361D, G364D, K365M, V368F, T371I, L374P, S376I, T384M, R385W, R385K, E387G, D390N, A392T, N393D, N393S, N393K, A394S, A394D, A394V, V395L, D397V, G398A, M403I, G406R, E407K, E407G, T408P, T408A, T408I, K410E, G411S, G411A, Q421K, A423A, A423A, R426W, R426Q, E427A, E427N, A431T, R449C, I457V, G458D, A459V, V460M, A468V, A468G, A470D, T477A, R479C, R479H, S485F, R486W, R486L, R488Q, R490W, I494T, A495T, A495V, R498C, R498H, A503V, R504L, Q505E, V506I, R510Q, G511R, G511E, R518S, R531C, R532W, R532Q, E538D, G540R, D550V, V552M, G557A, R559G, R559P, N566K, M568V, R569Q, R569L, Q58X, E174X, W201X, E241X, R270X, E440X, R486X, Q501X, L508X, R510X, E538X, R559X. These mutations are described in Canu et.al., Blood Cells, Molecules and Diseases 2016, 57, pp. 100-109. In an embodiment, the mutant PKR is selected from G332S, G364D, T384M, K410E, R479H, R479K, R486W, R532W, R510Q, and R490W. In certain embodiments, the mutant PKR is selected from A468V, A495V, I90N, T408I, and Q421K, and R498H. In certain embodiments, the mutant PKR is R532W, K410E, or R510Q. In certain embodiments, the mutant PKR is R510Q, R486W, or R479H.

In other aspects, provided are methods of treating a disease selected from hemolytic anemia, sickle cell disease, thalassemia, hereditary spherocytosis, hereditary elliptocytosis, abetalipoproteinemia, Bassen-Komzweig syndrome, and paroxysmal nocturnal hemoglobinuria in a subject in need thereof, comprising administering to the subject an effective amount of crystalline Form A, B, C, D, E, F, G, H, I, J, K, L, or M, or a pharmaceutical composition thereof. Also provided is crystalline Form A, B, C, D, E, F, G, H, I, J, K, L, or M, or a pharmaceutical composition thereof for use in treating disease selected from hemolytic anemia, sickle cell disease, thalassemia, hereditary spherocytosis, hereditary elliptocytosis, abetalipoproteinemia, Bassen-Komzweig syndrome, and paroxysmal nocturnal hemoglobinuria in a subject. Further provided is the use of crystalline Form A, B, C, D, E, F, G, H, I, J, K, L, or M, or a pharmaceutical composition thereof in the manufacture of a medicament for treating a disease selected from hemolytic anemia, sickle cell disease, thalassemia, hereditary spherocytosis, hereditary elliptocytosis, abetalipoproteinemia, Bassen-Komzweig syndrome, and paroxysmal nocturnal hemoglobinuria in a subject in need thereof. In one aspect, the disease to be treated is hemolytic anemia.

In other aspects, provided herein are methods for treating thalassemia (e.g., beta-thalassemia or non-transfusion-dependent thalassemia) in a subject in need thereof, comprising administering to the subject an effective amount of crystalline Form A, B, C, D, E, F, G, H, I, J, K, L, or M, or a pharmaceutical composition thereof. Also provided is crystalline Form A, B, C, D, E, F, G, H, I, J, K, L, or M, or a pharmaceutical composition thereof for use in treating thalassemia (e.g., beta-thalassemia or non-transfusion-dependent thalassemia). Further provided is the use of crystalline Form A, B, C, D, E, F, G, H, I, J, K, L, or M, or a pharmaceutical composition thereof in the manufacture of a medicament for treating thalassemia (e.g., beta-thalassemia or non-transfusion-dependent thalassemia).

In certain embodiments, the subject is an adult subject with thalassemia. In certain embodiments, the subject has thalassemia such as P-thalassemia intermedia, Hb E β-thalassemia, α-thalassemia (Hb H disease), or β-thalassemia with mutations of 1 or more α genes. In certain embodiments, the subject has beta-thalassemia or non-transfusion-dependent thalassemia. In certain embodiments, the subject is an adult male subject with thalassemia such as beta-thalassemia or non-transfusion-dependent thalassemia. In certain embodiments, the subject is a female subject with thalassemia such as beta-thalassemia or non-transfusion-dependent thalassemia. In certain embodiments, the subject is an adult female subject with thalassemia such as beta-thalassemia or non-transfusion-dependent thalassemia. In certain embodiments, the subject has a hemoglobin concentration of less than or equal to 6.0 g/dL. In certain embodiments, the subject has a hemoglobin concentration of less than or equal to 7.0 g/dL. In certain embodiments, the subject has a hemoglobin concentration of less than or equal to 8.0 g/dL. In certain embodiments, the subject has a hemoglobin concentration of less than or equal to 9.0 g/dL. In certain aspects, the subject having non-transfusion-dependent thalassemia does not have a known history (e.g., has been diagnosed in the past) of Hb S or Hb C forms of thalassemia. In certain embodiments, the term “non-transfusion dependent” thalassemia refers to subjects with thalassemia having no more than 4 (e.g. five) units of RBCs transfused during a 24-week period up to the first day of administration of a crystalline or amorphous form described herein and/or no RBC transfusions in the 8 weeks prior to the first day of administration of a crystalline or amorphous form described herein.

In other aspects, provided herein are methods for increasing the lifetime of red blood cells (RBCs) in a subject in need thereof comprising administering to the subject an effective amount of crystalline Form A, B, C, D, E, F, G, H, I, J, K, L, or M, or a pharmaceutical composition thereof. Also provided is crystalline Form A, B, C, D, E, F, G, H, I, J, K, L, or M, or a pharmaceutical composition thereof for use in increasing the lifetime of red blood cells (RBCs) in a subject in need thereof. Further provided is the use of crystalline Form A, B, C, D, E, F, G, H, I, J, K, L, or M, or a pharmaceutical composition thereof in the manufacture of a medicament for increasing the lifetime of red blood cells (RBCs). In one aspect, crystalline Form A, B, C, D, E, F, G, H, I, J, K, L, or M, or a pharmaceutical composition thereof is added directly to whole blood or packed red blood cells extracorporeally.

In other aspects, provided herein are methods for regulating 2,3-diphosphoglycerate levels in blood in a subject in need thereof comprising contacting blood with an effective amount of crystalline Form A, B, C, D, E, F, G, H, I, J, K, L, or M, or a pharmaceutical composition thereof. Also provided is crystalline Form A, B, C, D, E, F, G, H, I, J, K, L, or M, or a pharmaceutical composition thereof for use in regulating 2,3-diphosphoglycerate levels in blood in a subject in need thereof. Further provided is the use of crystalline Form A, B, C, D, E, F, G, H, I, J, K, L, or M, or a pharmaceutical composition thereof in the manufacture of a medicament for regulating 2,3-diphosphoglycerate levels in blood.

In other aspects, provided herein are methods for treating anemia in a subject in need thereof comprising administering to the subject an effective amount of crystalline Form A, B, C, D, E, F, G, H, I, J, K, L, or M, or a pharmaceutical composition thereof. Also provided is crystalline Form A, B, C, D, E, F, G, H, I, J, K, L, or M, or a pharmaceutical composition thereof for use in treating anemia in a subject in need thereof. Further provided is the use of crystalline Form A, B, C, D, E, F, G, H, I, J, K, L, or M, or a pharmaceutical composition thereof in the manufacture of a medicament for treating anemia. In one aspect, the anemia to be treated is dyserythropoietic anemia.

In certain embodiments, the anemia is a dyserythropoietic anemia such as congenital dyserythropoietic anemia type I, II, III, or IV. In certain embodiments, the anemia is hemolytic anemia. In certain embodiments, the hemolytic anemia is a congenital and/or hereditary form of hemolytic anemia such as PKD, sickle cell disease, thalassemias (e.g. alpha or beta or non-transfusion-dependent thalassemia), hereditary spherocytosis, hereditary elliptocytosis), paroxysmal nocturnal hemoglobinuria, abeta-liproteinemia (Bassen-Kornzweig syndrome). In certain embodiments, the hemolytic anemia is acquired hemolytic anemia such as autoimmune hemolytic anemia, drug-induced hemolytic anemia. In certain embodiments, the hemolytic anemia is anemia as part of a multi-system disease, such as the anemia of Congenital Erythropoietic Purpura, Fanconi, Diamond-Blackfan.

As used herein, the term “anemia” refers to a deficiency of red blood cells (RBCs) and/or hemoglobin. As used herein, anemia includes all types of clinical anemia, for example (but not limited to): microcytic anemia, iron deficiency anemia, hemoglobinopathies, heme synthesis defect, globin synthesis defect, sideroblastic defect, normocytic anemia, anemia of chronic disease, aplastic anemia, hemolytic anemia, macrocytic anemia, megaloblastic anemia, pernicious anemia, dimorphic anemia, anemia of prematurity, Fanconi anemia, hereditary spherocytosis, sickle cell disease, warm autoimmune hemolytic anemia, cold agglutinin hemolytic anemia, osteopetrosis, thalassemia, and myelodysplastic syndrome.

In certain embodiments, anemia can be diagnosed on a complete blood count. In certain embodiments, anemia can be diagnosed based on the measurement of one or more markers of hemolysis (e.g. RBC count, hemoglobin, reticulocytes, schistocytes, lactate Dehydrogenase (LDH), haptoglobin, bilirubin, and ferritin) and/or hemosiderinuria mean corpuscular volume (MCV) and/or red cell distribution width (RDW). In the context of the present invention, anemia is present if an individual has a hemoglobin (Hb) less than the desired level, for example, the Hb concentration of less than 14 g/dL, more preferably of less than 13 g/dL, more preferably of less than 12 g/dL, more preferably of less than 11 g/dL, or most preferably of less than 10 g/dL.

In certain embodiments, provided herein is a method of increasing the amount of hemoglobin in a subject by administering an effective amount of crystalline Form A, B, C, D, E, F, G, H, I, J, K, L, or M, or a pharmaceutical composition thereof as described herein. In certain embodiments, also provided herein is a method of increasing the amount of hemoglobin in a subject having thalassemia comprising administering to the subject an effective amount of crystalline Form A, B, C, D, E, F, G, H, I, J, K, L, or M, or a pharmaceutical composition thereof. Further provided is a method of increasing the amount of hemoglobin in subjects having non-transfusion-dependent thalassemia comprising administering an effective amount of crystalline Form A, B, C, D, E, F, G, H, I, J, K, L, or M, or a pharmaceutical composition thereof as described herein to the subject. In certain embodiments, the provided methods increase hemoglobin concentration in the subject. In certain embodiments, the provided methods increase Hb concentration to a desired level, for example, above 10 g/dL, more preferably above 11 g/dL, more preferably above 12 g/dL, more preferably above 13 g/dL, or most preferably above 14 g/dL. In certain embodiments, the provided methods increase Hb concentration by at least about 0.5 g/dL. In certain embodiments, the provided methods increase Hb concentration by at least about 1.0 g/dL. In certain embodiments, the provided methods increase Hb concentration by at least about 1.5 g/dL. In certain embodiments, the provided methods increase Hb concentration by at least about 2.0 g/dL. In certain embodiments, the provided methods increase Hb concentration by at least about 2.5 g/dL. In certain embodiments, the provided methods increase Hb concentration by at least about 3.0 g/dL. In certain embodiments, the provided methods increase Hb concentration by at least about 3.5 g/dL. In certain embodiments, the provided methods increase Hb concentration by at least about 4.0 g/dL. In certain embodiments, the provided methods increase Hb concentration by at least about 4.5 g/dL. In certain embodiments, the provided methods increase Hb concentration by at least about 5.0 g/dL. In certain embodiments, the provided methods increase Hb concentration by at least about 5.5 g/dL. In certain embodiments, the provided methods increase Hb concentration by at least about 6.0 g/dL. In certain embodiments, the increase in Hb concentration is determined from baseline at one or more assessment between week 1 and week 20 (e.g., between week 2 and week 15, between week 3 and week 15, and between week 4 and week 12) of treatment with an effective amount of crystalline Form A, B, C, D, E, F, G, H, I, J, K, L, or M, or a pharmaceutical composition thereof as described herein. In certain embodiments, the provided methods increase Hb concentration as described above in female subjects having thalassemia (e.g., beta-thalassemia or non-transfusion-dependent thalassemia). In certain embodiments, the provided methods increase Hb concentration from baseline to about 12 g/dL in female subjects having thalassemia (e.g., beta-thalassemia or non-transfusion-dependent thalassemia). In certain embodiments, the provided methods increase Hb concentration as described above in male subjects having thalassemia (e.g., beta-thalassemia or non-transfusion-dependent thalassemia). In certain embodiments, the provided methods increase Hb concentration from baseline to about 13 g/dL in male subjects having thalassemia (e.g., beta-thalassemia or non-transfusion-dependent thalassemia).

In some aspects, provided herein are methods for treating hemolytic anemia in a subject in need thereof comprising administering to the subject an effective amount of crystalline Form A, B, C, D, E, F, G, H, I, J, K, L, or M, or a pharmaceutical composition thereof. Also provided is crystalline Form A, B, C, D, E, F, G, H, I, J, K, L, or M, or a pharmaceutical composition thereof for use in treating hemolytic anemia in a subject in need thereof. Further provided is the use of crystalline Form A, B, C, D, E, F, G, H, I, J, K, L, or M, or a pharmaceutical composition thereof in the manufacture of a medicament for treating hemolytic anemia. In one aspect, the hemolytic anemia to be treated is hereditary and/or congenital hemolytic anemia, acquired hemolytic anemia, or anemia as part of a multi-system disease.

In some aspects, provided herein are methods for treating sickle cell disease in a subject in need thereof comprising administering to the subject an effective amount of crystalline Form A, B, C, D, E, F, G, H, I, J, K, L, or M, or a pharmaceutical composition thereof. Also provided is crystalline Form A, B, C, D, E, F, G, H, I, J, K, L, or M, or a pharmaceutical composition thereof for use in treating sickle cell disease in a subject in need thereof. Further provided is the use of crystalline Form A, B, C, D, E, F, G, H, I, J, K, L, or M, or a pharmaceutical composition thereof in the manufacture of a medicament for treating sickle cell disease.

In some aspects, provided herein are methods for treating thalassemia, hereditary spherocytosis, hereditary elliptocytosis, abetalipoproteinemia or Bassen-Komzweig syndrome, sickle cell disease, paroxysmal nocturnal hemoglobinuria, acquired hemolytic anemia, or anemia of chronic diseases in a subject in need thereof comprising administering to the subject an effective amount of crystalline Form A, B, C, D, E, F, G, H, I, J, K, L, or M, or a pharmaceutical composition thereof. Also provided is crystalline Form A, B, C, D, E, F, G, H, I, J, K, L, or M, or a pharmaceutical composition thereof for use in treating thalassemia, hereditary spherocytosis, hereditary elliptocytosis, abetalipoproteinemia or Bassen-Kornzweig syndrome, sickle cell disease, paroxysmal nocturnal hemoglobinuria, acquired hemolytic anemia, or anemia in a subject in need thereof. Further provided is the use of crystalline Form A, B, C, D, E, F, G, H, I, J, K, L, or M, or a pharmaceutical composition thereof in the manufacture of a medicament for treating thalassemia, hereditary spherocytosis, hereditary elliptocytosis, abetalipoproteinemia or Bassen-Komzweig syndrome, sickle cell disease, paroxysmal nocturnal hemoglobinuria, acquired hemolytic anemia, or anemia.

In some aspects, provided herein are methods for activating wild-type or mutant PKR in red blood cells in a subject in need thereof comprising administering to the subject an effective amount of crystalline Form A, B, C, D, E, F, G, H, I, J, K, L, or M, or a pharmaceutical composition thereof. Also provided is crystalline Form A, B, C, D, E, F, G, H, I, J, K, L, or M, or a pharmaceutical composition thereof for use in activating wild-type or mutant PKR in red blood cells in a subject in need thereof. Further provided is the use of crystalline Form A, B, C, D, E, F, G, H, I, J, K, L, or M, or a pharmaceutical composition thereof in the manufacture of a medicament for activating wild-type or mutant PKR in red blood cells.

The provided crystalline Form A, B, C, D, E, F, G, H, I, J, K, L, or M, and pharmaceutical compositions described herein are activators of PKR mutants having lower activities compared to the wild type, thus are useful for methods of the present disclosure. Such mutations in PKR can affect enzyme activity (catalytic efficiency), regulatory properties (modulation by fructose bisphosphate (FBP)/ATP), and/or thermostability of the enzyme. Examples of such mutations are described in Valentini et al, JBC 2002. Some examples of the mutants that are activated by the compounds described herein include G332S, G364D, T384M, R479H, R479K, R486W, R532W, R510Q, and R490W. Without being bound by theory, in certain embodiments, the compounds described herein affect the activities of PKR mutants by activating FBP non-responsive PKR mutants, restoring thermostability to mutants with decreased stability, or restoring catalytic efficiency to impaired mutants. The activating activity of the present compounds against PKR mutants may be tested following a method described in the Examples. Compounds described herein are also activators of wild type PKR.

In certain embodiments, the provided crystalline Form A, B, C, D, E, F, G, H, I, J, K, L, or M, and pharmaceutical compositions described herein increase the affinity of PKR to phosphoenolpyruvate (PEP). In certain embodiments, the provided crystalline Form A, B, C, D, E, F, G, H, I, J, K, L, or M, and pharmaceutical compositions described herein restore the ability of RBCs to cover PEP and ADP to pyruvate and ATP.

In certain embodiments, provided herein are methods of reducing transfusion frequency of a subject with PKD comprising administering to the subject crystalline Form A, B, C, D, E, F, G, H, I, J, K, L, or M, and pharmaceutical compositions described herein. In certain embodiments, crystalline Form A is administered. In certain embodiments, the transfusion frequency is reduced by at least 5% in the number of RBC units transfused over at least 15 weeks. In certain embodiments, the transfusion frequency is reduced by at least 10% in the number of RBC units transfused over at least 15 weeks. In certain embodiments, the transfusion frequency is reduced by at least 15% in the number of RBC units transfused over at least 15 weeks. In certain embodiments, the transfusion frequency is reduced by at least 20% in the number of RBC units transfused over at least 15 weeks. In certain embodiments, the transfusion frequency is reduced by at least 25% in the number of RBC units transfused over at least 15 weeks. In certain embodiments, the transfusion frequency is reduced by at least 30% in the number of RBC units transfused over at least 15 weeks. In certain embodiments, the transfusion frequency is reduced by at least 35% in the number of RBC units transfused over at least 15 weeks. In certain embodiments, the transfusion frequency is reduced by at least 40% in the number of RBC units transfused over at least 20 weeks. In certain embodiments, the transfusion frequency is reduced by at least 5% in the number of RBC units transfused over at least 20 weeks. In certain embodiments, the transfusion frequency is reduced by at least 10% in the number of RBC units transfused over at least 20 weeks. In certain embodiments, the transfusion frequency is reduced by at least 15% in the number of RBC units transfused over at least 20 weeks. In certain embodiments, the transfusion frequency is reduced by at least 20% in the number of RBC units transfused over at least 20 weeks. In certain embodiments, the transfusion frequency is reduced by at least 25% in the number of RBC units transfused over at least 20 weeks. In certain embodiments, the transfusion frequency is reduced by at least 30% in the number of RBC units transfused over at least 20 weeks. In certain embodiments, the transfusion frequency is reduced by at least 35% in the number of RBC units transfused over at least 20 weeks. In certain embodiments, the transfusion frequency is reduced by at least 40% in the number of RBC units transfused over at least 20 weeks.

Is some aspects, provided herein are methods of evaluating a subject, the method comprising: administering to the subject crystalline Form A, B, C, D, E, F, G, H, I, J, K, L, or M, or a pharmaceutical composition thereof; and acquiring a value for the level of the crystalline or amorphous form, the level of 2,3-diphosphoglycerate (2,3-DPG), the level of adenosine triphosphate (ATP), or the activity of PKR in the subject, to thereby evaluate the subject. In some aspects, the value for the level is acquired by analyzing the plasma concentration of crystalline or amorphous form. In some aspects, the level of 2,3-DPG is acquired by analyzing the blood concentration of 2,3-DPG. In some aspects, the level of ATP is acquired by analyzing the blood concentration of ATP. In some aspects, the activity of PKR is acquired by analyzing the blood concentration of a ¹³C-label in the blood. In some aspects, the analysis is performed by sample analysis of bodily fluid. In some aspects, the bodily fluid is blood. In some aspects, the analysis is performed by mass spectroscopy. In some aspects, the analysis is performed by LC-MS.

In some aspects, provided herein are methods of evaluating a subject, the method comprising acquiring, the value for the level of crystalline Form A, B, C, D, E, F, G, H, I, J, K, L, or M, or a pharmaceutical composition thereof, the level of 2,3-DPG, the level of ATP, or the activity of PKR in a subject that has been treated with crystalline Form A, B, C, D, E, F, G, H, I, J, K, L, or M, or a pharmaceutical composition thereof, to thereby evaluate the subject. In some aspects, acquiring comprises receiving a sample from the subject. In some aspects, acquiring comprises transmitting the value to another party. In some aspects, the other party is the party that administered crystalline Form A, B, C, D, E, F, G, H, I, J, K, L, or M, or a pharmaceutical composition thereof.

In some aspects, provided herein are methods of treating a subject, the method comprising: administering to the subject an effective amount of crystalline Form A, B, C, D, E, F, G, H, I, J, K, L, or M, or a pharmaceutical composition thereof; and acquiring a value for the level of the crystalline or amorphous form, the level of 2,3-diphosphoglycerate (2,3-DPG), the level of adenosine triphosphate (ATP), or the activity of PKR in the subject, to thereby treat the subject.

In some aspects, an effective amount of a disclosed form (crystalline Form A, B, C, D, E, F, G, H, I, J, L, or M) can be administered to cells in culture, e.g. in vitro or ex vivo, or to a subject, e.g., in vivo, to treat, prevent, and/or diagnose a variety of disorders, including those described herein below.

In one aspect, the disclosed compositions, methods of treatment, and uses thereof, comprising a disclosed form (crystalline Form A, B, C, D, E, F, G, H, I, J, L, or M) further comprise the administration or use of folic acid. The administration or use of folic acid can be prior to, during, and/or following the administration or use of a crystalline or amorphous form described herein. In one aspect, however, the folic acid is administered or used prior to and/or concurrently with a disclosed form (crystalline Form A, B, C, D, E, F, G, H, I, J, L, or M). Thus, in one aspect, provided herein is a method for treating a condition described herein (e.g., PKD, anemia such as hemolytic anemia, acquired hemolytic anemia, and sickle cell anemia, thalassemia (e.g., beta-thalassemia, alpha-thalassemia, non-transfusion dependent thalassemia, etc.), sickle cell disease, hereditary spherocytosis, hereditary elliptocytosis, abetalipoproteinemia, Bassen-Komzweig syndrome, and paroxysmal nocturnal hemoglobinuria); increasing the lifetime of RBCs; regulating 2,3-diphosphoglycerate levels in blood; activating wild-type or mutant PKR in red blood cells; increasing the amount of hemoglobin; evaluating the level of 2,3-diphosphoglycerate (2,3-DPG), the level of adenosine triphosphate (ATP), or the activity of PKR; evaluating the level of 2,3-diphosphoglycerate (2,3-DPG), the level of adenosine triphosphate (ATP), or the activity of PKR; in a subject in need thereof, comprising administering to the subject an effective of a disclosed form (crystalline Form A, B, C, D, E, F, G, H, I, J, L, or M) and folic acid.

In aspects where folic acid is administered or used prior to a disclosed form (crystalline Form A, B, C, D, E, F, G, H, I, J, L, or M), the folic acid may be used at least 5 days, at least 10 days, at least 15 days, at least 20 days, or at least 25 days prior to the administration or use of disclosed form. In one aspect, the folic acid is administered or used at least 20, at least 21, at least 22, at least 23, at least 24, or at least 25 days prior to the administration or use of disclosed form. In another aspect, the folic acid is administered at least 21 days prior to the administration or use of disclosed form. In another aspect, the folic acid is administered or used from 1 to 30 days prior to the administration or use of disclosed form. In another aspect, the folic acid is administered or used from 5 to 25 days prior to the administration or use of disclosed form. In another aspect, the folic acid is administered or used from 10 to 30 days prior to the administration or use of disclosed form. In another aspect, the folic acid is administered or used from 10 to 25 days prior to the administration or use of disclosed form. In another aspect, the folic acid is administered or used from 15 to 25 days prior to the administration or use of disclosed form. In another aspect, the folic acid is administered or used from 20 to 25 days prior to the administration or use of disclosed form.

Specific amounts of folic acid to be administered or used with a disclosed form will vary depending upon the subject to be treated and the particular mode of administration. In certain aspects, the effective amount of folic acid is about 0.1 mg to about 10 mg daily. In certain aspects, the effective amount of folic acid is at least 0.1, 0.2, 0.3, 0.4, 0.5, 0.6, 0.7, 0.8, 0.9 or 1.0 mg daily. In one aspect, the effective amount of folic acid is at least 0.8 mg daily or at least 1.0 mg daily.

The amount of folic acid is intended to be combined with any amount of a disclosed form described herein. Thus, in certain aspects, provided herein is a method for treating a condition described herein (e.g., PKD, anemia such as hemolytic anemia, acquired hemolytic anemia, and sickle cell anemia, thalassemia (e.g., beta-thalassemia, alpha-thalassemia, non-transfusion dependent thalassemia, etc.), sickle cell disease, hereditary spherocytosis, hereditary elliptocytosis, abetalipoproteinemia, Bassen-Komzweig syndrome, and paroxysmal nocturnal hemoglobinuria); increasing the lifetime of RBCs; regulating 2,3-diphosphoglycerate levels in blood; activating wild-type or mutant PKR in red blood cells; increasing the amount of hemoglobin; evaluating the level of 2,3-diphosphoglycerate (2,3-DPG), the level of adenosine triphosphate (ATP), or the activity of PKR; evaluating the level of 2,3-diphosphoglycerate (2,3-DPG), the level of adenosine triphosphate (ATP), or the activity of PKR; in a subject in need thereof, comprising administering to the subject an effective amount of a disclosed form described herein (crystalline Form A, B, C, D, E, F, G, H, I, J, L, or M) and folic acid, wherein the folic acid is administered prior to and/or concurrently with the disclosed form (e.g., at least 21 days prior), the disclosed form (e.g. Form A) is administered in an amount of 5, 20, or 50 mg BID and wherein the folic acid is administered in an amount of at least 0.8 mg/day .

### EXEMPLIFICATION

As depicted in the Examples below, crystalline and salt forms were prepared according to the following general procedures.

The crystalline hemisulfate salt of Compound (I) sesquihydrate was obtained by following the procedures set forth in International Application No. PCT/US2018/062197, and for ease of reference, is defined herein as “Starting Material”. The XRPD pattern and peak listings for “Starting Material” of International Application No. PCT/US2018/062197 are shown in FIG. 1 and Table 23, respectively.

List of Abbreviations
Solvents

| Name | Abbreviation |
|---|---|
| 1-propanol | 1-PA |
| 2-propanol | IPA |
| Acetonitrile | ACN |
| Benzyl Alcohol | BA |
| Dichloromethane | DCM |
| Dimethyl Sulfoxide | DMSO |
| Ethanol | EtOH |
| Ethyl Acetate | EtOAc |
| Isopropyl Acetate | IPAc |
| Methanol | MeOH |
| Methyl Acetate | MeOAc |
| Methyl Butyl Ketone | MBK |
| Methyl Ethyl Ketone | MEK |
| Methyl Isobutyl Ketone | MIBK |
| N,N-Dimethylacetamide | DMAc |
| N,N-Dimethylformamide | DMF |
| N-Methyl Pyrrolidone | NMP |
| tert-Butyl Methyl Ether | MtBE |
| Tetrahydrofuran | THF |
| Trifluoroacetic Acid | TFA |
| Trifluoroethanol | TFE |
| Units | |
| Name | Abbreviation |
| Celsius | C |
| Degrees | ° |
| Equivalents | eq. |
| Gram | g |
| Hour | hr |
| Kelvin | K |
| Liters | L |
| Milligrams | mg |
| Milliliters | mL |
| Minute | min |
| Second | sec |
| volume | vol. |
| Watt | W |
| weight | wt. |

### 1. Instrument and Methodology Details

### X-Rav Powder Diffraction (XRPD)

The Rigaku Smart-Lab X-ray diffraction system was configured for reflection Bragg- Brentano geometry using a line source X-ray beam. The x-ray source was a Cu Long Fine Focus tube that was operated at 40 kV and 44 ma. That source provided an incident beam profile at the sample that changes from a narrow line at high angles to a broad rectangle at low angles. Beam conditioning slits were used on the line X-ray source to ensure that the maximum beam size was less than 10 mm both along the line and normal to the line. The Bragg-Brentano geometry was a para-focusing geometry controlled by passive divergence and receiving slits with the sample itself acting as the focusing component for the optics. The inherent resolution of Bragg-Brentano geometry was governed in part by the diffractometer radius and the width of the receiving slit used. Typically, the Rigaku Smart-Lab was operated to give peak widths of 0.1 °2θ or less. The axial divergence of the X-ray beam was controlled by 5.0-degree Soller slits in both the incident and diffracted beam paths.

Powder samples were prepared in a low background Si holder using light manual pressure to keep the sample surfaces flat and level with the reference surface of the sample holder. Each sample was analyzed from 2 to 40 °2θ using a continuous scan of 6 °2θ per minute with an effective step size of 0.02 °2θ.

### Differential Scanning Calorimetry (DSC)

DSC analyses were carried out using a TA Instruments Q2000 instrument. The instrument temperature calibration was performed using indium. The DSC cell was kept under a nitrogen purge of ~50 mL per minute during each analysis. The sample was placed in a standard, crimped, aluminum pan and was heated from 25° C. to 350° C. at a rate of 10° C. per minute.

### Thermogravimetric (TG) Analysis

The TG analysis was carried out using a TA Instruments Q50 instrument. The instrument balance was calibrated using class M weights and the temperature calibration was performed using alumel. The nitrogen purge was ~40 mL per minute at the balance and ~60 mL per minute at the furnace. Each sample was placed into a pre- tared platinum pan and heated from 20° C. to 350° C. at a rate of 10° C. per minute.

### HPLC Analyses

HPLC analyses were carried out on an Agilent 1100 series instrument equipped with a UV detector using the following materials and operating parameters:

| | |
|---|---|
| column | Waters Xbridge C18 (150 mm x 4.6 mm, 3.5 µm) PN 186003034 |
| column temperature | 40° C. |
| detector wavelength | 220 nm |
| mobile phase A | 25 mM (NH₄)₂HPO₄ and 2.5 mM NH₄H₂PO₄ in water (pH 6.8 +/- 0.1 |
| mobile phase B | ACN/MeOH (80/20, v/v) |
| injection volume | 5 µL |
| flow rate | 1.5 mL/min |
| run time | 32 min |

The following gradient was used:

| Time (min) | A% |
|---|---|
| 0 | 70 |
| 15 | 55 |
| 20 | 15 |
| 25 | 15 |
| 25.1 | 70 |
| 32 | 70 |

### Nuclear Magnetic Resonance (NMR) Spectroscopy

The ¹H NMR spectra were acquired on a Bruker DRX-500 spectrometer located at the Chemistry Department of Purdue University. Samples were prepared by dissolving material in DMSO-d₆. The solutions were filtered and placed into individual 5-mm NMR tubes for subsequent spectral acquisition. The temperature controlled (298K) ¹H NMR spectra acquired on the DRX-500 utilized a 5-mm cryoprobe operating at an observing frequency of 499.89 MHz.

### 2. Salt Screen

The Starting Material was mixed with various acids under various conditions in attempts to generate crystalline salts. Nine samples were found to exhibit an XRPD pattern suggestive of new phase formation. That is, the patterns contained peaks that did not arise from the Starting Material or the corresponding acid. The acids used in those experiments were benzenesulfonic, fumaric, gentisic, hydrochloric, maleic, malonic, phosphoric, L-tartaric, and p-toluenesulfonic. The screening conditions and the XRPD Patterns are summarized in the table below. The characterizations of Forms A to M are presented below.

| Acid | Conditions^{a} | XRPD Pattern^{b} |
|---|---|---|
| besylic | C, acetone, -15° C., 7 days | NC |
| | SL, THF, RT, 7 days | Form A |
| | SL, ACN, RT, 3 days | |
| ethane sulfonic | C, acetone, -15° C., 7 days | NC |
| | SL, THF, RT, 7 days | NC |
| | SL, ACN, RT; Et₂O AS, turbid. Clear, C, RT➔-15° C. | NC |
| fumaric | C, acetone, -15° C., 2 days | Form B |
| | C, THF, -15° C., 7 days | Form C |
| | SL, ACN, RT, 3 days | Form B |
| gentisic | C, acetone, -15° C., 7 days | NC |
| | C, THF, -15° C., 7 days | Form E |
| | SL, ACN, RT, 3 days | Form D |
| glutamic | SL, acetone, RT, 7 days | NC + acid |
| | C, THF, -15° C., 7 days | acid + pks |
| | SL, ACN, RT, 3 days | Starting Material + acid |
| HCl | C, acetone, -15° C., 7 days | Form G + another phase |
| | C, acetone, -15° C., 3 days | Form G |
| | SL, THF, RT, 7 days | Form F |
| | SL, ACN, RT; some solids dissolved. Et₂O AS, turbid | Form F + another phase |
| | SL, ACN, RT, 3 days | Form F |
| 2-naphthoic, 1-hydroxy | C, acetone, -15° C., 7 days | NC |
| | C, THF, -15° C., 7 days | NC |
| | SL, ACN, RT; some solids dissolved. Et₂O AS, turbid. Clear, C, RT7➔15° C. | NC |
| maleic | C, acetone, -15° C., 7 days | NC |
| | C, THF, -15° C., 7 days | Form H + NC |
| | SL, ACN, RT, 3 days | Form H |
| malonic | C, acetone, -15° C., 7 days | NC |
| | C, THF, -15° C., 7 days | NC |
| | SL, ACN, RT; solids dissolved. Et₂O AS, turbid | Form I |
| | SL, ACN, RT, 3 days | |
| phosphoric | SL, acetone, RT, 7 days | Form J + Form K |
| | SL, acetone, RT, 3 days | Form J |
| | SL, THF, RT, 7 days | Form K |
| | SL, ACN, RT, 3 days | NC + pks |
| L-tartaric | C, acetone, -15° C., 7 days | LC- |
| | C, THF, -15° C., 7 days | new phase A + NC |
| | SL, ACN, RT, 3 days | Starting Material+ new phase B |
| tosylic | C, acetone, -15° C., 7 days | NC |
| | SL, THF, RT, 7 days | Form M |
| | SL, ACN, RT; Et₂O AS, turbid. Clear, C, RT➔15° C. | NC |

a. ACN = acetonitrile, C = cool, Et₂O = ethyl ether, E = evaporation, RT = room temperature, SL = slurry, THF = tetrahydrofuran
b. LC = low crystallinity, NC = non-crystalline, pks = peaks

### 3. Preparation and Characterization of Crystalline Salt Forms of Compound (I)

### Example 1: Crystalline Besylate Salt Form A

A mixture of 78.4 mg (0.174 mmol) of Starting Material, 27.7 mg (0.175 mmol) of besylic acid, and 1 mL of acetonitrile, consisting of a slurry of solid in liquid, was agitated at ambient temperature for 3 days, followed by centrifugation and decantation of the liquid phase. Next, the solid was allowed to dry in the air to give the salt form, characterized as crystalline besylate salt Form A by XRPD. The XRPD for Form A is shown by FIG. 1 and the peak listings are shown in Table 1. A combined TGA and DSC is shown by FIG. 2.

TABLE 1

Crystalline Besylate Salt Form A

| Peak No. | Position (°2θ) | Relative Intensity | Peak No. | Position (°2θ) | Relative Intensity |
|---|---|---|---|---|---|
| 1 | 6.3652 | 3.5 | 35 | 24.7705 | 2.14 |
| 2 | 8.8013 | 3.61 | 36 | 24.9554 | 11.69 |
| 3 | 11.5428 | 1.43 | 37 | 25.1658 | 4.84 |
| 4 | 12.1404 | 5.05 | 38 | 25.7069 | 3.66 |
| 5 | 12.7087 | 18.58 | 39 | 25.8161 | 12.79 |
| 6 | 13.1813 | 6.43 | 40 | 26.7072 | 6.54 |
| 7 | 13.4748 | 1.01 | 41 | 27.1184 | 5.67 |
| 8 | 13.8294 | 16.69 | 42 | 27.2939 | 6.4 |
| 9 | 14.457 | 18.19 | 43 | 27.6419 | 3.12 |
| 10 | 14.6472 | 5.53 | 44 | 27.8832 | 6.19 |
| 11 | 15.4393 | 43.4 | 45 | 28.3278 | 1.39 |
| 12 | 15.9101 | 97.98 | 46 | 28.4735 | 17.87 |
| 13 | 16.4491 | 8.6 | 47 | 29.1461 | 10.96 |
| 14 | 16.7796 | 3.4 | 48 | 29.5131 | 1.5 |
| 15 | 17.6971 | 7.15 | 49 | 29.9133 | 4.29 |
| 16 | 17.9642 | 11.89 | 50 | 30.3231 | 2.27 |
| 17 | 18.4042 | 25.65 | 51 | 30.6278 | 7.93 |
| 18 | 18.4424 | 9.37 | 52 | 30.8132 | 11.8 |
| 19 | 18.6981 | 15.3 | 53 | 31.0985 | 0.8 |
| 20 | 18.8569 | 11.31 | 54 | 31.9266 | 2.07 |
| 21 | 19.0408 | 43.26 | 55 | 32.0785 | 2.06 |
| 22 | 19.6217 | 6.94 | 56 | 32.4863 | 3.55 |
| 23 | 19.897 | 1.37 | 57 | 32.8641 | 2.99 |
| 24 | 20.1769 | 17.78 | 58 | 33.4138 | 8.54 |
| 25 | 20.3326 | 13.74 | 59 | 33.8662 | 2.2 |
| 26 | 20.6894 | 25.04 | 60 | 34.229 | 1.12 |
| 27 | 21.295 | 50.5 | 61 | 34.8013 | 3.72 |
| 28 | 21.9489 | 6.34 | 62 | 35.3621 | 4.68 |
| 29 | 22.0753 | 10.85 | 63 | 35.7836 | 2.28 |
| 30 | 23.2953 | 100 | 64 | 35.9834 | 3.75 |
| 31 | 23.6054 | 24.39 | 65 | 36.4468 | 0.84 |
| 32 | 23.6835 | 14.63 | 66 | 37.1467 | 8.13 |
| 33 | 24.0846 | 19.52 | 67 | 37.7787 | 6.05 |
| 34 | 24.4644 | 25.49 | 68 | 38.4998 | 1.42 |
| | | | 69 | 39.0558 | 2.56 |

### Example 2: Crystalline Fumarate Salt Form B

A mixture of 76.0 mg (0.169 mmol) of Starting Material, 20.1 mg (0.173 mmol) of fumaric acid, and 1 mL of acetonitrile, consisting of a slurry of solid in liquid, was agitated at ambient temperature for 3 days, followed by centrifugation and decantation of the liquid phase. Next, the solid was allowed to dry in the air to give the salt form, characterized as crystalline fumarate salt Form B by XRPD. The XRPD for Form B is shown by FIG. 3 and the peak listings are shown in Table 2. A combined TGA and DSC is shown by FIG. 4.

TABLE 2

Crystalline Fumarate Salt Form B

| Peak No. | Position (°2θ) | Relative Intensity |
|---|---|---|
| 1 | 4.0815 | 30.78 |
| 2 | 8.179 | 33.72 |
| 3 | 10.8397 | 18.8 |
| 4 | 11.5688 | 1.65 |
| 5 | 12.2611 | 7.09 |
| 6 | 12.9358 | 3.18 |
| 7 | 14.2604 | 7.99 |
| 8 | 14.7608 | 28.55 |
| 9 | 15.3218 | 14.51 |
| 10 | 15.9396 | 9 |
| 11 | 16.4005 | 2.66 |
| 12 | 17.8085 | 50.51 |
| 13 | 18.3698 | 4.89 |
| 14 | 19.1754 | 1.01 |
| 15 | 20.528 | 16.95 |
| 16 | 21.3197 | 33.34 |
| 17 | 21.722 | 22.68 |
| 18 | 22.5983 | 2.44 |
| 19 | 22.865 | 9.01 |
| 20 | 23.2597 | 9.26 |
| 21 | 23.7346 | 1.17 |
| 22 | 24.6695 | 100 |
| 23 | 24.9565 | 41.72 |
| 24 | 26.0395 | 3.91 |
| 25 | 26.9952 | 7.23 |
| 26 | 27.524 | 4 |
| 27 | 28.1772 | 4.89 |
| 28 | 28.8891 | 10.98 |
| 29 | 29.3989 | 0.73 |
| 30 | 30.2621 | 3.41 |
| 31 | 31.8287 | 1.53 |
| 32 | 33.0613 | 34.36 |
| 33 | 35.7671 | 2.31 |
| 34 | 37.1847 | 2.09 |
| 35 | 37.9421 | 2.11 |

### Example 3: Crystalline Fumarate Salt Form C

A mixture of 77.9 mg (0.173 mmol) of Starting Material and 20.4 mg (0.176 mmol) of fumaric acid was dissolved in 7 mL of a mixture of THF and acetone. The solution was kept in a freezer (about -15° C.) for 3 days, followed by centrifugation and decantation of the liquid phase. Next, the solid was allowed to dry in the air to give the salt form, characterized as crystalline fumarate salt Form C by XRPD. The XRPD for Form C is shown by FIG. 5 and the peak listings are shown in Table 3. A combined TGA and DSC is shown by FIG. 6.

TABLE 3

Crystalline Fumarate Salt Form C
Peak Position Relative No. (°2θ) Intensity

| | | |
|---|---|---|
| 1 | 4.1168 | 6.64 |
| 2 | 6.901 | 12.23 |
| 3 | 8.5388 | 38.56 |
| 4 | 9.2048 | 4.72 |
| 5 | 10.4331 | 17.36 |
| 6 | 10.8701 | 4.46 |
| 7 | 11.5199 | 58.31 |
| 8 | 12.3412 | 23.11 |
| 9 | 13.8578 | 15.28 |
| 10 | 15.6486 | 98.12 |
| 11 | 16.057 | 100 |
| 12 | 16.5499 | 17.01 |
| 13 | 17.825 | 43.93 |
| 14 | 18.1741 | 53.17 |
| 15 | 18.6645 | 89.92 |
| 16 | 19.1268 | 7.94 |
| 17 | 19.7544 | 23.37 |
| 18 | 20.6668 | 12.02 |
| 19 | 21.0018 | 44.02 |
| 20 | 21.2655 | 66.54 |
| 21 | 22.1437 | 11.95 |
| 22 | 23.3056 | 24.98 |
| 23 | 24.1174 | 75.92 |
| 24 | 25.1737 | 91.59 |
| 25 | 27.822 | 38.96 |
| 26 | 29.1361 | 44.44 |
| 27 | 30.415 | 12.82 |
| 28 | 32.6867 | 14.26 |
| 29 | 33.2223 | 5.72 |
| 30 | 36.2319 | 6.15 |

### Example 4: Crystalline Gentisate Salt Form D

A mixture of 78.0 mg (0.175 mmol) of Starting Material, 26.7 mg (0.173 mmol) of gentisic acid, and 1 mL of acetonitrile, consisting of a slurry of solid in liquid, was agitated at ambient temperature for 3 days, followed by centrifugation and decantation of the liquid phase. Next, the solid was allowed to dry in the air to give the salt form, characterized as crystalline gentisate salt Form D by XRPD. The XRPD for Form D is shown by FIG. 7 and the peak listings are shown in Table 4. A combined TGA and DSC is shown by FIG. 8.

TABLE 4

Crystalline Gentisate Salt Form D

| Peak No. | Position (°2θ) | Relative Intensity |
|---|---|---|
| 1 | 4.5358 | 64.33 |
| 2 | 9.0287 | 24.92 |
| 3 | 9.8396 | 1.67 |
| 4 | 10.7376 | 12.77 |
| 5 | 10.937 | 3.1 |
| 6 | 11.5365 | 30.64 |
| 7 | 11.8978 | 24.01 |
| 8 | 12.4593 | 5.36 |
| 9 | 13.2057 | 56.72 |
| 10 | 13.5516 | 45.5 |
| 11 | 13.9414 | 5.99 |
| 12 | 14.4868 | 36.32 |
| 13 | 15.2189 | 6.4 |
| 14 | 16.118 | 53.96 |
| 15 | 16.3332 | 11.87 |
| 16 | 16.9181 | 100 |
| 17 | 18.1489 | 54.29 |
| 18 | 18.2913 | 36.12 |
| 19 | 19.2892 | 8.39 |
| 20 | 19.7162 | 24.61 |
| 21 | 20.4209 | 14.79 |
| 22 | 21.2659 | 19.34 |
| 23 | 21.7293 | 67.54 |
| 24 | 21.9259 | 35.21 |
| 25 | 22.4471 | 64.42 |
| 26 | 22.6787 | 42.13 |
| 27 | 22.819 | 20.49 |
| 28 | 22.997 | 51.72 |
| 29 | 23.4562 | 16.02 |
| 30 | 23.9498 | 66.74 |
| 31 | 24.4552 | 22.11 |
| 32 | 25.2684 | 30.73 |
| 33 | 26.5953 | 10.61 |
| 34 | 27.0654 | 45.46 |
| 35 | 27.3071 | 45.41 |
| 36 | 28.0333 | 2.47 |
| 37 | 29.4515 | 25.64 |
| 38 | 30.0123 | 8.24 |
| 39 | 30.897 | 3.79 |
| 40 | 31.9632 | 17.89 |
| 41 | 32.9995 | 3.12 |
| 42 | 33.7287 | 1.7 |
| 43 | 34.0139 | 2.47 |
| 44 | 34.8749 | 2.62 |
| 45 | 36.2107 | 4.72 |
| 46 | 37.0994 | 3.46 |
| 47 | 37.5899 | 5.55 |
| 48 | 38.3109 | 5.17 |
| 49 | 38.6384 | 2.08 |

### Example 5: Crystalline Gentisate Salt Form E

A mixture of 76.0 mg (0.169 mmol) of Starting Material and 26.0 mg (0.169 mmol) of gentisic acid was dissolved in 7 mL of a mixture of THF and acetonitrile. The solution was kept in a freezer (about -15° C.) for 6 days, during which time crystallization occurred. The mixture was removed from the freezer and left in an uncapped vial at ambient temperature until all the solvent had evaporated. The resulting solid salt was characterized as crystalline gentisate salt Form E by XRPD. The XRPD for c Form E is shown by FIG. 9 and the peak listings are shown in Table 5. A combined TGA and DSC is shown by FIG. 10.

TABLE 5

Crystalline Gentisate Salt Form E

| Peak No. | Position (°2θ) | Relative Intensity |
|---|---|---|
| 1 | 4.5871 | 31.95 |
| 2 | 9.1013 | 22.69 |
| 3 | 9.7963 | 1.55 |
| 4 | 10.8778 | 34 |
| 5 | 11.4392 | 12.07 |
| 6 | 11.8151 | 45.1 |
| 7 | 12.6438 | 12.92 |
| 8 | 13.4587 | 74.13 |
| 9 | 14.2189 | 8.06 |
| 10 | 14.464 | 11.75 |
| 11 | 15.327 | 15.65 |
| 12 | 16.4531 | 76.27 |
| 13 | 16.6297 | 11.28 |
| 14 | 17.9655 | 50.02 |
| 15 | 18.1587 | 87.34 |
| 16 | 18.8334 | 5.94 |
| 17 | 20.4808 | 23.62 |
| 18 | 20.7848 | 11.73 |
| 19 | 21.1495 | 30.5 |
| 20 | 21.5996 | 100 |
| 21 | 21.7259 | 9.17 |
| 22 | 22.1143 | 80.22 |
| 23 | 22.6915 | 83.1 |
| 24 | 23.4612 | 22.53 |
| 25 | 23.6731 | 61.49 |
| 26 | 24.1299 | 38.38 |
| 27 | 24.5255 | 6.58 |
| 28 | 25.7547 | 45.53 |
| 29 | 26.706 | 15.59 |
| 30 | 26.9223 | 12.31 |
| 31 | 27.3159 | 41.99 |
| 32 | 27.8109 | 5.74 |
| 33 | 28.5656 | 13.34 |
| 34 | 29.3811 | 18.95 |
| 35 | 30.8676 | 15.03 |
| 36 | 31.9456 | 4.2 |
| 37 | 32.5325 | 8.01 |
| 38 | 33.3235 | 4.49 |
| 39 | 34.0282 | 2.48 |
| 40 | 36.2653 | 4.3 |
| 41 | 37.2075 | 6.08 |
| 42 | 37.8026 | 1.13 |
| 43 | 38.1718 | 2.22 |
| 44 | 39.69 | 4.79 |

### Example 6: Crystalline Hydrochloride Salt Form F

A mixture of 78.7 mg (0.175 mmol) of Starting Material, 17.7 mg (0.180 mmol) of 37% aqueous hydrochloric acid, and 1 mL of acetonitrile, consisting of a slurry of solid in liquid, was agitated at ambient temperature for 3 days, followed by centrifugation and decantation of the liquid phase. Next, the solid was allowed to dry in the air to give the salt form, characterized as crystalline hydrochloride salt Form F by XRPD. The XRPD for Form F is shown by FIG. 11 and the peak listings are shown in Table 6. A combined TGA and DSC is shown by FIG. 12.

TABLE 6

Crystalline Hydrochloride Salt Form F

| Peak No. | Position (°2θ) | Relative Intensity | Peak No. | Position (°2θ) | Relative Intensity |
|---|---|---|---|---|---|
| 1 | 4.5382 | 0.27 | 37 | 23.9013 | 23.95 |
| 2 | 6.7 | 3.61 | 38 | 25.0513 | 18.88 |
| 3 | 7.3677 | 2.93 | 39 | 25.4694 | 15.22 |
| 4 | 9.9856 | 8.07 | 40 | 25.6187 | 27.12 |
| 5 | 10.4344 | 0.78 | 41 | 26.0692 | 10.32 |
| 6 | 10.7934 | 3.31 | 42 | 26.1848 | 3.94 |
| 7 | 11.3495 | 48.77 | 43 | 26.8599 | 8.68 |
| 8 | 11.6375 | 5.01 | 44 | 27.35 | 1.41 |
| 9 | 11.8977 | 9.71 | 45 | 27.6531 | 25.3 |
| 10 | 12.6529 | 3.06 | 46 | 28.2604 | 19.07 |
| 11 | 13.3838 | 2.53 | 47 | 28.9164 | 10.08 |
| 12 | 14.3448 | 12.47 | 48 | 29.4052 | 19.43 |
| 13 | 14.9374 | 2.85 | 49 | 30.4407 | 8.03 |
| 14 | 15.3499 | 49.35 | 50 | 30.6809 | 6.89 |
| 15 | 15.7597 | 100 | 51 | 30.8814 | 8.04 |
| 16 | 15.929 | 28.13 | 52 | 31.1423 | 1.04 |
| 17 | 17.1518 | 3.95 | 53 | 31.6914 | 6.78 |
| 18 | 18.002 | 36.72 | 54 | 32.1312 | 8.51 |
| 19 | 18.1818 | 6.16 | 55 | 32.4324 | 5.96 |
| 20 | 18.4962 | 12.88 | 56 | 33.1401 | 5.67 |
| 21 | 18.9999 | 47.21 | 57 | 33.3027 | 6.29 |
| 22 | 19.2573 | 3.2 | 58 | 33.5023 | 5.3 |
| 23 | 19.4998 | 2.14 | 59 | 33.7809 | 6.6 |
| 24 | 19.6606 | 5.41 | 60 | 34.4203 | 2.87 |
| 25 | 19.8803 | 44.48 | 61 | 34.7208 | 1.99 |
| 26 | 20.0006 | 25.1 | 62 | 35.2066 | 1.39 |
| 27 | 20.6798 | 3.41 | 63 | 35.7378 | 5.01 |
| 28 | 21.2063 | 10.29 | 64 | 35.8834 | 5.04 |
| 29 | 21.3792 | 10.46 | 65 | 36.8051 | 3.02 |
| 30 | 21.6944 | 8.32 | 66 | 37.2906 | 2.21 |
| 31 | 21.9669 | 7.65 | 67 | 37.4436 | 1.93 |
| 32 | 22.1318 | 7.27 | 68 | 37.673 | 3.61 |
| 33 | 22.5135 | 15.71 | 69 | 38.5813 | 3.73 |
| 34 | 22.7773 | 47.66 | 70 | 38.9267 | 3.59 |
| 35 | 23.3584 | 62.72 | 71 | 39.7014 | 1.35 |
| 36 | 23.626 | 34.86 | - | - | |

### Example 7: Crystalline Hydrochloride Salt Form G

A mixture of 75 mg (0.17 mmol) of Starting Material and 16.9 mg (0.172 mmol) of 37% aqueous hydrochloric acid was dissolved in about 49 mL of acetone. The solution was kept in a freezer (about -15° C.) for 6 days, during which time crystallization occurred. The mixture was removed from the freezer and left in an uncapped vial at ambient temperature until all the solvent had evaporated. The resulting solid salt was characterized as crystalline hydrochloride salt Form G by XRPD. The XRPD for Form G is shown by FIG. 13 and the peak listings are shown in Table 7. A combined TGA and DSC is shown by FIG. 14.

TABLE 7

Crystalline Hydrochloride Salt Form G

| Peak No. | Position (°2θ) | Relative Intensity |
|---|---|---|
| 1 | 5.5854 | 27.68 |
| 2 | 7.6571 | 54.79 |
| 3 | 8.3491 | 9.19 |
| 4 | 8.7596 | 12.03 |
| 5 | 10.1188 | 37.94 |
| 6 | 11.1215 | 17.15 |
| 7 | 12.5097 | 2.77 |
| 8 | 12.8231 | 4.39 |
| 9 | 13.2143 | 4.13 |
| 10 | 13.7021 | 9.81 |
| 11 | 14.3569 | 13.98 |
| 12 | 14.9536 | 11.41 |
| 13 | 15.4479 | 14.51 |
| 14 | 16.5519 | 25.33 |
| 15 | 17.3085 | 32.17 |
| 16 | 17.4982 | 100 |
| 17 | 17.9975 | 16.01 |
| 18 | 18.7569 | 31.26 |
| 19 | 19.8338 | 10.16 |
| 20 | 20.1829 | 4.83 |
| 21 | 20.7035 | 8.57 |
| 22 | 20.8715 | 36.75 |
| 23 | 21.958 | 5.56 |
| 24 | 22.3452 | 3.28 |
| 25 | 22.8761 | 61.12 |
| 26 | 23.3756 | 10.48 |
| 27 | 24.0025 | 10.15 |
| 28 | 24.2815 | 8.43 |
| 29 | 25.1963 | 33.26 |
| 30 | 25.6836 | 46.81 |
| 31 | 26.2981 | 2.76 |
| 32 | 27.9336 | 16.52 |
| 33 | 30.2102 | 15.73 |
| 34 | 31.8759 | 4.28 |
| 35 | 32.717 | 3 |
| 36 | 33.3708 | 2.66 |
| 37 | 36.0522 | 6.23 |
| 38 | 38.7065 | 5.6 |

### Example 8: Crystalline Maleate Salt Form H

A mixture of 76.4 mg (0.170 mmol) of Starting Material, 19.8 mg (0.171 mmol) of maleic acid, and 1 mL of acetonitrile, consisting of a slurry of solid in liquid, was agitated at ambient temperature for 3 days, followed by centrifugation and decantation of the liquid phase. Next, the solid was allowed to dry in the air to give the salt form, characterized as crystalline maleate salt Form H by XRPD. The XRPD for Form H is shown by FIG. 15 and the peak listings are shown in Table 8. A combined TGA and DSC is shown by FIG. 16.

TABLE 8

Crystalline Maleate Salt Form H

| Peak No. | Position (°2θ) | Relative Intensity | Peak No. | Position (°2θ) | Relative Intensity |
|---|---|---|---|---|---|
| 1 | 7.837 | 21.01 | 36 | 25.0487 | 18.77 |
| 2 | 8.1289 | 2.4 | 37 | 25.2063 | 11.64 |
| 3 | 9.0523 | 10.84 | 38 | 25.5959 | 11.82 |
| 4 | 10.4617 | 15.37 | 39 | 26.0277 | 11.42 |
| 5 | 10.6036 | 23.29 | 40 | 26.1233 | 21.54 |
| 6 | 10.7864 | 39.54 | 41 | 26.244 | 40.25 |
| 7 | 11.771 | 13.52 | 42 | 26.77 | 2.65 |
| 8 | 13.3154 | 3.4 | 43 | 27.2805 | 5.05 |
| 9 | 14.2508 | 5.81 | 44 | 28.0028 | 12.96 |
| 10 | 15.4377 | 8.01 | 45 | 28.3645 | 3.55 |
| 11 | 15.7625 | 26.71 | 46 | 28.6799 | 5.67 |
| 12 | 15.8693 | 11.31 | 47 | 28.8866 | 9.55 |
| 13 | 16.1244 | 8.88 | 48 | 29.4556 | 5.73 |
| 14 | 16.253 | 21.6 | 49 | 29.8945 | 12.06 |
| 15 | 16.5477 | 25.29 | 50 | 30.082 | 6.71 |
| 16 | 16.8637 | 6.49 | 51 | 30.2467 | 22.2 |
| 17 | 17.07 | 0.9 | 52 | 30.7398 | 3.34 |
| 18 | 17.4174 | 6.96 | 53 | 31.1837 | 1.08 |
| 19 | 17.959 | 14.92 | 54 | 31.3478 | 3.63 |
| 20 | 18.3088 | 24.71 | 55 | 32.241 | 3.51 |
| 21 | 18.6761 | 11.79 | 56 | 32.4997 | 3.75 |
| 22 | 18.9144 | 19.68 | 57 | 32.8186 | 3.62 |
| 23 | 19.4107 | 28.89 | 58 | 33.0976 | 4.6 |
| 24 | 19.9074 | 39.95 | 59 | 33.2186 | 6.09 |
| 25 | 20.0213 | 33.53 | 60 | 33.3554 | 2.83 |
| 26 | 20.8282 | 31.24 | 61 | 33.8415 | 6.7 |
| 27 | 20.9622 | 6.96 | 62 | 34.3829 | 1.71 |
| 28 | 21.4415 | 100 | 63 | 34.8428 | 3.83 |
| 29 | 21.6071 | 70.52 | 64 | 35.7576 | 1.4 |
| 30 | 22.2099 | 16.65 | 65 | 36.1073 | 1.68 |
| 31 | 22.3454 | 11.43 | 66 | 36.3714 | 1.28 |
| 32 | 22.6868 | 7.63 | 67 | 36.698 | 1.68 |
| 33 | 23.1376 | 18.7 | 68 | 37.727 | 8.41 |
| 34 | 24.0062 | 3.85 | 69 | 38.5393 | 6.63 |
| 35 | 24.4648 | 47.7 | 70 | 39.0052 | 2.26 |

### Example 9: Crystalline Malonate Salt Form I

A mixture of 77.2 mg (0.171 mmol) of Starting Material, 18.1 mg (0.174 mmol) of malonic acid, and 1 mL of acetonitrile, consisting of a slurry of solid in liquid, was agitated at ambient temperature for 3 days, followed by centrifugation and decantation of the liquid phase. Next, the solid was allowed to dry in the air to give the salt form, characterized as crystalline malonate salt Form I by XRPD. The XRPD for Form I is shown by FIG. 17 and the peak listings are shown in Table 9. A combined TGA and DSC is shown by FIG. 18.

TABLE 9

Crystalline Malonate Salt Form I

| Peak No. | Position (°2θ) | Relative Intensity | Peak No. | Position (°2θ) | Relative Intensity |
|---|---|---|---|---|---|
| 1 | 7.068 | 0.91 | 30 | 25.0814 | 27.1 |
| 2 | 8.5271 | 3.25 | 31 | 25.9436 | 4.54 |
| 3 | 9.1527 | 4.88 | 32 | 26.4536 | 6.1 |
| 4 | 10.1972 | 2.08 | 33 | 26.8813 | 6.72 |
| 5 | 10.7018 | 6.94 | 34 | 27.4443 | 6.57 |
| 6 | 11.3322 | 4.16 | 35 | 27.6532 | 3.5 |
| 7 | 11.4259 | 5.87 | 36 | 27.8622 | 2.11 |
| 8 | 12.1213 | 14.78 | 37 | 28.0828 | 1.87 |
| 9 | 12.7827 | 0.44 | 38 | 28.6083 | 4.73 |
| 10 | 13.3498 | 1.31 | 39 | 29.1444 | 0.79 |
| 11 | 14.1228 | 5.55 | 40 | 29.5786 | 1.89 |
| 12 | 14.9044 | 1.85 | 41 | 30.1236 | 4.36 |
| 13 | 15.824 | 2.62 | 42 | 30.7465 | 7.59 |
| 14 | 16.0841 | 13.88 | 43 | 31.2797 | 0.91 |
| 15 | 16.989 | 13.92 | 44 | 31.6326 | 0.74 |
| 16 | 18.1879 | 10.03 | 45 | 31.8461 | 1.85 |
| 17 | 18.2381 | 18.68 | 46 | 32.2955 | 2.76 |
| 18 | 18.5184 | 5.59 | 47 | 32.8808 | 0.27 |
| 19 | 18.676 | 0.99 | 48 | 33.4685 | 0.42 |
| 20 | 18.9248 | 3.94 | 49 | 34.3503 | 4.4 |
| 21 | 20.2709 | 20.57 | 50 | 34.9254 | 1.53 |
| 22 | 20.7352 | 27.75 | 51 | 35.8128 | 0.92 |
| 23 | 21.2911 | 100 | 52 | 36.254 | 6.71 |
| 24 | 21.474 | 18.61 | 53 | 37.8783 | 1.63 |
| 25 | 22.0377 | 13.3 | 54 | 38.056 | 1.02 |
| 26 | 22.7857 | 2.44 | 55 | 38.4346 | 2.28 |
| 27 | 23.441 | 11.26 | 56 | 39.0796 | 1.11 |
| 28 | 23.7669 | 4.89 | 57 | 39.4218 | 0.86 |
| 29 | 24.8061 | 6.99 | - | - | - |

### Example 10: Crystalline Phosphate Salt Form J

A mixture of 75 mg (0.17 mmol) of Starting Material, 19.7 mg (0.171 mmol) of 85% aqueous phosphoric acid, and about 49 mL of acetone, consisting of a slurry of solid in liquid, was agitated at ambient temperature for 3 days, followed by centrifugation and decantation of the liquid phase. Next, the solid was allowed to dry in the air to give the salt form, characterized as crystalline phosphate salt Form J by XRPD. The XRPD for Form J is shown by FIG. 19 and the peak listings are shown in Table 10. A combined TGA and DSC is shown by FIG. 20.

TABLE 10

Crystalline Phosphate Salt Form J

| Peak No. | Position (°2θ) | Relative Intensity |
|---|---|---|
| 1 | 4.7437 | 5.53 |
| 2 | 5.9483 | 3.35 |
| 3 | 9.069 | 12.97 |
| 4 | 9.3861 | 13.81 |
| 5 | 11.0253 | 8.11 |
| 6 | 11.9479 | 16.69 |
| 7 | 12.8457 | 27.11 |
| 8 | 13.4099 | 23.81 |
| 9 | 14.2242 | 25.01 |
| 10 | 14.9684 | 24.07 |
| 11 | 15.5203 | 8.01 |
| 12 | 16.5156 | 7.42 |
| 13 | 17.416 | 42.07 |
| 14 | 18.1494 | 15.91 |
| 15 | 18.5425 | 6.41 |
| 16 | 20.018 | 49.15 |
| 17 | 20.1718 | 11.29 |
| 18 | 20.7455 | 18.62 |
| 19 | 21.1667 | 8.29 |
| 20 | 21.8573 | 30.48 |
| 21 | 22.1141 | 100 |
| 22 | 22.5429 | 30.14 |
| 23 | 23.0382 | 6.81 |
| 24 | 23.4636 | 11.23 |
| 25 | 24.21 | 26.28 |
| 26 | 24.689 | 17.01 |
| 27 | 25.2194 | 2.67 |
| 28 | 25.5527 | 8.49 |
| 29 | 26.6198 | 13.42 |
| 30 | 27.6812 | 4.7 |
| 31 | 29.5608 | 4.87 |
| 32 | 30.1066 | 2.07 |
| 33 | 30.402 | 2.86 |
| 34 | 31.2553 | 2.82 |
| 35 | 32.828 | 2.51 |
| 36 | 33.5882 | 5.31 |
| 37 | 34.4311 | 1.83 |
| 38 | 35.0063 | 2.69 |
| 39 | 37.8323 | 4.23 |

### Example 11: Crystalline Phosphate Salt Form K

A mixture of 77.3 mg (0.172 mmol) of Starting Material, 20.1 mg (0.174 mmol) of 85% aqueous phosphoric acid, and 6 mL of THF, consisting of a slurry of solid in liquid, was agitated at ambient temperature for 3 days, followed by centrifugation and decantation of the liquid phase. Next, the solid was allowed to dry in the air to give the salt form, characterized as crystalline phosphate salt Form K by XRPD. The XRPD for Form K is shown by FIG. 21 and the peak listings are shown in Table 11. A combined TGA and DSC is shown by FIG. 22.

TABLE 11

Crystalline Phosphate Salt Form K

| Peak No. | Position (°2θ) | Relative Intensity |
|---|---|---|
| 1 | 7.5128 | 9.85 |
| 2 | 11.3619 | 6.64 |
| 3 | 12.5583 | 12.19 |
| 4 | 12.9266 | 11.82 |
| 5 | 13.367 | 100 |
| 6 | 14.2381 | 3.12 |
| 7 | 14.4822 | 3.55 |
| 8 | 14.9896 | 15.46 |
| 9 | 15.4161 | 38.64 |
| 10 | 16.3612 | 11.2 |
| 11 | 17.9385 | 13.26 |
| 12 | 18.6584 | 10.67 |
| 13 | 20.2958 | 38.29 |
| 14 | 21.7793 | 46.63 |
| 15 | 22.4479 | 6.15 |
| 16 | 22.7639 | 1.76 |
| 17 | 23.1639 | 1.95 |
| 18 | 24.1272 | 8.77 |
| 19 | 24.862 | 14.72 |
| 20 | 25.1908 | 9.51 |
| 21 | 27.5811 | 13 |
| 22 | 28.1215 | 9.66 |
| 23 | 29.1389 | 7.07 |
| 24 | 30.3675 | 6.07 |
| 25 | 33.4117 | 6.5 |
| 26 | 34.5123 | 4.99 |
| 27 | 36.2312 | 4.45 |
| 28 | 38.5878 | 1.83 |
| 29 | 39.1967 | 4.99 |

### Example 12: Crystalline Tartrate Salt Form L

A mixture of 77.8 mg (0.173 mmol) of Starting Material and 25.9 mg (0.173 mmol) of L-tartaric acid was dissolved in about 49 mL of acetone. The solution was kept in a freezer (about -15° C.) for 7 days, during which time crystallization did not occur. The solution was removed from the freezer and left in an uncapped vial at ambient temperature until all the solvent had evaporated. The resulting solid salt was characterized as crystalline tartrate salt Form L by XRPD. The XRPD for Form L is shown by FIG. 23 and the peak listings are shown in Table 12. A combined TGA and DSC is shown by FIG. 24.

TABLE 12

Crystalline Tartrate Salt Form L

| Peak No. | Position (°2θ) | Relative Intensity |
|---|---|---|
| 1 | 3.7832 | 20.39 |
| 2 | 7.3971 | 100 |
| 3 | 8.9811 | 23.53 |
| 4 | 9.2626 | 8.72 |
| 5 | 11.1836 | 26.57 |
| 6 | 11.7113 | 7.51 |
| 7 | 12.0156 | 9.49 |
| 8 | 13.2385 | 29.26 |
| 9 | 13.6649 | 82.32 |
| 10 | 14.4127 | 69.39 |
| 11 | 14.8347 | 54.71 |
| 12 | 15.5251 | 9.69 |
| 13 | 17.0235 | 29.41 |
| 14 | 17.867 | 11.55 |
| 15 | 18.8867 | 13.03 |
| 16 | 19.2339 | 5.02 |
| 17 | 19.5899 | 17.31 |
| 18 | 19.9939 | 33.99 |
| 19 | 20.3193 | 19.81 |
| 20 | 21.1133 | 4.65 |
| 21 | 21.5434 | 39.44 |
| 22 | 22.195 | 38.59 |
| 23 | 22.6504 | 58.57 |
| 24 | 22.9011 | 47.24 |
| 25 | 23.3794 | 40.67 |
| 26 | 24.0469 | 14.62 |
| 27 | 24.45 | 3.67 |
| 28 | 24.8979 | 2.38 |
| 29 | 25.2533 | 3.59 |
| 30 | 26.5591 | 6.55 |
| 31 | 27.0853 | 16.47 |
| 32 | 27.7034 | 39.49 |
| 33 | 28.943 | 8.54 |
| 34 | 33.6311 | 20.16 |
| 35 | 34.6923 | 18.46 |
| 36 | 3.7832 | 20.39 |
| 37 | 7.3971 | 100 |
| 38 | 8.9811 | 23.53 |
| 39 | 9.2626 | 8.72 |

### Example 13: Crystalline Tosylate Salt Form M

A mixture of 75.6 mg (0.168 mmol) of Starting Material, 32.3 mg (0.188 mmol) of tosylic acid, and 6 mL of THF, consisting of a slurry of solid in liquid, was agitated at ambient temperature for 3 days, followed by centrifugation and decantation of the liquid phase. Next, the solid was allowed to dry in the air to give the salt form, characterized as crystalline tosylate salt Form M by XRPD. The XRPD for Form M is shown by FIG. 25 and the peak listings are shown in Table 13. A combined TGA and DSC is shown by FIG. 26.

TABLE 13

Crystalline Tosylate Salt Form M

| Peak No. | Position (°2θ) | Relative Intensity | Peak No. | Position (°2θ) | Relative Intensity |
|---|---|---|---|---|---|
| 1 | 5.5861 | 13.75 | 38 | 23.7448 | 12.33 |
| 2 | 6.4763 | 8.36 | 39 | 24.2059 | 22.72 |
| 3 | 7.0728 | 9.38 | 40 | 24.5454 | 47.85 |
| 4 | 8.4736 | 3.58 | 41 | 25.6816 | 4.09 |
| 5 | 8.9252 | 10.8 | 42 | 25.9193 | 3.05 |
| 6 | 12.1377 | 17.93 | 43 | 26.1758 | 6.91 |
| 7 | 12.3983 | 6.91 | 44 | 26.4749 | 2.73 |
| 8 | 12.9289 | 26.38 | 45 | 26.6019 | 9.2 |
| 9 | 13.4989 | 24.47 | 46 | 26.7228 | 7.12 |
| 10 | 13.7897 | 22.49 | 47 | 26.8835 | 11.38 |
| 11 | 14.1169 | 2.82 | 48 | 27.1246 | 2.15 |
| 12 | 14.4265 | 11.8 | 49 | 27.4401 | 1.28 |
| 13 | 14.4477 | 4.39 | 50 | 27.6674 | 14.73 |
| 14 | 14.5477 | 14.43 | 51 | 27.804 | 14.59 |
| 15 | 15.6614 | 93.51 | 52 | 28.3811 | 5.72 |
| 16 | 15.8899 | 29.87 | 53 | 28.7418 | 6.34 |
| 17 | 16.3151 | 2.59 | 54 | 29.0692 | 11.2 |
| 18 | 16.9587 | 12.04 | 55 | 29.2121 | 10.55 |
| 19 | 17.122 | 16.54 | 56 | 30.2207 | 3.38 |
| 20 | 17.3615 | 11.63 | 57 | 30.5188 | 10.77 |
| 21 | 17.7689 | 41.54 | 58 | 31.1428 | 18.82 |
| 22 | 17.9641 | 20.57 | 59 | 31.3219 | 5.38 |
| 23 | 18.107 | 19.73 | 60 | 31.6302 | 15.39 |
| 24 | 18.8077 | 32.06 | 61 | 31.8955 | 4.01 |
| 25 | 19.0327 | 25.03 | 62 | 33.4847 | 12.53 |
| 26 | 19.1482 | 15.59 | 63 | 34.0396 | 1.55 |
| 27 | 19.4306 | 2.18 | 64 | 34.3862 | 2.98 |
| 28 | 19.7584 | 24.13 | 65 | 34.7754 | 3.33 |
| 29 | 19.8968 | 19.82 | 66 | 35.5681 | 2.73 |
| 30 | 20.0265 | 23.83 | 67 | 35.7259 | 2.07 |
| 31 | 20.3564 | 8.7 | 68 | 35.9242 | 0.73 |
| 32 | 21.7558 | 27.46 | 69 | 36.3823 | 5.19 |
| 33 | 22.0974 | 100 | 70 | 37.3168 | 5.61 |
| 34 | 22.3923 | 11.29 | 71 | 37.64 | 8.18 |
| 35 | 22.5692 | 10.4 | 72 | 38.0545 | 1.88 |
| 36 | 22.9467 | 11.68 | 73 | 39.1527 | 7.02 |
| 37 | 23.4809 | 15.53 | 74 | 39.3337 | 5.83 |

### Example 14: Purity and Stability of Crystalline Salt Forms

The chemical purity analyzed by HPLC and crystalline stability by XRPD for each of the crystalline salt Forms A to M prepared according to the procedures described in Examples 1-13 are summarized in Table 14. In short, these salt forms all yielded a chemical purity higher than 99%. The majority of the salt forms (Forms A, B, C, D, F, H, I, J, and M) maintained their original forms after a period of 7 days at an elevated temperature and relative humidity (e.g., 40° C./75% RH).

TABLE 14

| Crystalline Salt Form | HPLC Purity | Crystalline Stability |
|---|---|---|
| Crystalline Besylate Salt FormA | >99% | no change in XRPD pattern after 7 days at 40° C./75% RH |
| Crystalline Fumarate Salt Form B | >99% | no change in XRPD pattern after 7 days at 40° C./75% RH or after heating to 120° C. (removal of volatiles) |
| Crystalline Fumarate Salt Form C | >99% | no change in XRPD pattern after 7 days at 40° C./75% RH |
| Crystalline Gentisate Salt Form D | >99% | no change in XRPD pattern after 7 days at 40° C./75% RH |
| Crystalline Gentisate Salt Form E | >99% | significant changes in XRPD pattern after 7 days at 40° C./75% RH |
| Crystalline Hydrochloride Salt Form F | >99% | no change in XRPD pattern after 7 days at 40° C./75% RH or after heating to 130° C. (removal of volatiles) |
| Crystalline Hydrochloride Salt Form G | >99% | unique XRPD pattern after 7 days at 40° C./75% RH |
| Crystalline Maleate Salt Form H | >98% | no change in XRPD pattern after 7 days at 40° C./75% RH |
| Crystalline Malonate Salt Form I | >99% | no change in XRPD pattern after 7 days at 40° C./75% RH |
| Crystalline Phosphate Salt Form K | >98% | no change in XRPD pattern after 7 days at 40° C./75% RH |
| Crystalline Phosphate Salt Form J | >99% | additional peaks in XRPD pattern after 7 days at 40° C./75% RH, no change in XRPD pattern after heating to 175° C. (removal of volatiles) |
| Crystalline Tartrate Salt Form L | >99% | unique XRPD pattern after 7 days at 40° C./75% RH, becomes non-crystalline after heating to 150° C. (removal of volatiles) |
| Crystalline Tosylate Salt Form M | >99% | no change in XRPD pattern after 7 days at 40° C./75% RH |

While a number of embodiments have been described, the scope of this disclosure is to be defined by the appended claims, and not by the specific embodiments that have been represented by way of example. The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A besylate salt, fumarate salt, gentisate salt, hydrochloride salt, maleate salt, malonate salt, phosphate salt, tartrate salt, or tosylate salt of compound (I) represented by the following structural formula: 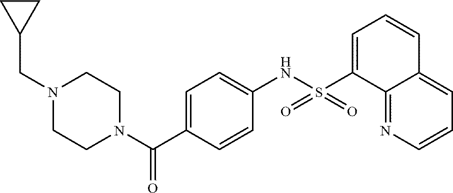 [SMILES: O=C(c1ccc(NS(=O)(=O)c2cccc3cccnc23)cc1)N1CCN(CC2CC2)CC1]
wherein the molar ratio between compound (I) and besylate acid, fumaric acid, gentisic acid, hydrochloric acid, maleic acid, malonic acid, phosphoric acid, tartaric acid, or tosylic acid is 1:1.

2. The besylate salt, fumarate salt, gentisate salt, hydrochloride salt, maleate salt, malonate salt, phosphate salt, tartrate salt, or tosylate salt of claim 1, wherein the besylate salt, fumarate salt, gentisate salt, hydrochloride salt, maleate salt, malonate salt, phosphate salt, tartrate salt, or tosylate salt are in crystalline form.

3. (canceled)

4. The besylate salt, fumarate salt, gentisate salt, hydrochloride salt, maleate salt, malonate salt, phosphate salt, tartrate salt, or tosylate salt of claim 2, wherein
the besylate crystalline form is crystalline Form A characterized by x-ray powder diffraction peaks at 2Θ angles (± 0.2°) 15.4°, 15.9°, 21.3°, and 23.3°;
the fumarate crystalline form is crystalline Form B characterized by x-ray powder diffraction peaks at 20 angles (± 0.2°) 17.8°, 24.7°, 25.0°, and 33.1°; or the fumarate salt crystalline form is crystalline Form C characterized by x-ray powder diffraction peaks at 20 angles (± 0.2°) 15.6°, 16.1°, 18.7°, and 25.2°;
the gentisate crystalline form is crystalline Form D characterized by x-ray powder diffraction peaks at 20 angles (± 0.2°) 16.9°, 21.7°, 22.4°, and 23.9°; or the gentisate crystalline form is crystalline Form E characterized by x-ray powder diffraction peaks at 20 angles (± 0.2°) 18.2°, 21.6°, 22.1°, and 22.7°;
the hydrochloride crystalline form is crystalline Form F characterized by x-ray powder diffraction peaks at 20 angles (± 0.2°) 11.3°, 15.3°, 15.8°, and 23.4°; or the hydrochloride crystalline form is crystalline Form G characterized by x-ray powder diffraction peaks at 20 angles (± 0.2°) 7.7°, 17.5°, 22.9°, and 25.7°;
the maleate crystalline form is crystalline Form H characterized by x-ray powder diffraction peaks at 20 angles (± 0.2°) 21.4°, 21.6°, 24.5°, and 26.2°; or the maleate crystalline form is crystalline Form I characterized by x-ray powder diffraction peaks at 20 angles (± 0.2°) 20.3°, 20.7°, 21.3°, and 25.1°;
the phosphate crystalline form is crystalline Form J characterized by x-ray powder diffraction peaks at 20 angles (± 0.2°) 17.4°. 20.0°. 21.9°. and 22.1°; or the phosphate crystalline form is crystalline Form K characterized by x-ray powder diffraction peaks at 20 angles (± 0.2°) 13.4°, 15.4°, 20.3°, and 21.8°;
the tartrate crystalline form is crystalline Form L characterized by x-ray powder diffraction peaks at 20 angles (± 0.2°) 7.4°, 13.7°, 14.4°, and 22.7°; and
the tosylate crystalline form is crystalline Form M characterized by x-ray powder diffraction peaks at 20 angles (± 0.2°) 15.7°, 17.8°, 22.1°, and 24.5°.

5. The besylate salt, fumarate salt, gentisate salt, hydrochloride salt, maleate salt, malonate salt, phosphate salt, tartrate salt, or tosylate salt of claim 4, wherein
crystalline Form A is characterized by x-ray powder diffraction peaks at 2Θ angles (± 0.2°) 15.4°, 15.9°, 21.3°, and 23.3°; and at least one, at least two, or at least three additional x-ray powder diffraction peaks at 2Θ angles (± 0.2°) selected from 18.4°, 19.0°, 20.7°, and 24.5°;
crystalline Form B is characterized by x-ray powder diffraction peaks at 20 angles (± 0.2°) 17.8°, 24.7°, 25.0°, and 33.1°; and at least one, at least two, or at least three additional x-ray powder diffraction peaks at 20 angles (± 0.2°) selected from 4.1°, 8.2°, 14.8°, and 21.3°;
crystalline Form C is characterized by x-ray powder diffraction peaks at 20 angles (± 0.2°) 15.6°. 16.1°, 18.7°, and 25.2°; and at least one, at least two, or at least three additional x-ray powder diffraction peak at 20 angles (± 0.2°) selected from 11.5°. 18.2°. 21.3°, and 24.1°;
crystalline Form D is characterized by x-ray powder diffraction peaks at 20 angles (± 0.2°) 16.9°, 21.7°, 22.4°, and 23.9°; and at least one, at least two, or at least three additional x-ray powder diffraction peaks at 20 angles (± 0.2°) selected from 4.5°, 13.2°, 16.1°, and 18.1°;
crystalline Form E is characterized by x-ray powder diffraction peaks at 20 angles (± 0.2°) 18.2°, 21.6°, 22.1°, and 22.7°; and at least one, at least two, or at least three additional x-ray powder diffraction peak at 20 angles (± 0.2°) selected from 13.5°, 16.5°, 18.0°, and 23.7°;
crystalline Form F is characterized by x-ray powder diffraction peaks at 20 angles (± 0.2°) 11.3°, 15.3°, 15.8°, and 23.4°; and at least one, at least two, or at least three additional x-ray powder diffraction peaks at 20 angles (± 0.2°) selected from 18.0°, 19.0°, 19.9°, and 22.8°;
crystalline Form G is characterized by x-ray powder diffraction peaks at 20 angles (± 0.2°) 7.7°, 17.5°, 22.9°, and 25.7°; and at least one, at least two, or at least three additional x-ray powder diffraction peaks at 20 angles (± 0.2°) selected from 10.1°, 17.3°, 20.9°, and 25.2°;
crystalline Form H is characterized by x-ray powder diffraction peaks at 20 angles (± 0.2°) 21.4°, 21.6°, 24.5°, and 26.2°; and at least one, at least two, or at least three additional x-ray powder diffraction peaks at 20 angles (± 0.2°) selected from 10.8°, 19.9°, 20.0°. and 20.8°;
crystalline Form I is characterized by x-ray powder diffraction peaks at 20 angles (± 0.2°) 20.3°, 20.7°, 21.3°, and 25.1 °; and at least one, at least two, or at least three additional x-ray powder diffraction peaks at 20 angles (± 0.2°) selected from 12.1°, 17.0°, 18.2°, and 21.5°;
crystalline Form J is characterized by x-ray powder diffraction peaks at 20 angles (± 0.2°) 17.4°, 20.0°, 21.9°, and 22.1°; and at least one, at least two, or at least three additional x-ray powder diffraction peaks at 20 angles (± 0.2°) selected from 12.8°, 14.2°, 22.5°, and 24.2°;
crystalline Form K is characterized by x-ray powder diffraction peaks at 20 angles (± 0.2°) 13.4°, 15.4°, 20.3°, and 21.8°; and at least one, at least two, or at least three additional x-ray powder diffraction peaks at 20 angles (± 0.2°) selected from 15.0°, 17.9°, 24.9°, and 27.6°;
crystalline Form L is characterized by x-ray powder diffraction peaks at 20 angles (± 0.2°) 7.4°, 13.7°. 14.4°, and 22.7°; and at least one, at least two, or at least three additional x-ray powder diffraction peaks at 20 angles (± 0.2°) selected from 14.8°, 22.9°, 23.4°, and 27.7°; and
crystalline Form M is characterized by x-ray powder diffraction peaks at 20 angles (± 0.2°) 15.7°, 17.8°. 22.1°, and 24.5°; and at least one, at least two, or at least three additional x-ray powder diffraction peaks at 20 angles (± 0.2°) selected from 12.9°, 15.9°, 18.8°, and 21.8°.

6. The besylate salt, fumarate salt, gentisate salt, hydrochloride salt, maleate salt, malonate salt, phosphate salt, tartrate salt, or tosylate salt of claim 5, wherein
crystalline Form A is characterized by x-ray powder diffraction peaks at 2Θ angles (± 0.2°) 15.4°, 15.9°, 18.4°, 19.0°, 20.7°, 21.3°, 23.3°, and 24.5°;
crystalline Form B is characterized by x-ray powder diffraction peaks at 20 angles (± 0.2°) 4.1°, 8.2°, 14.8°, 17.8°_{.} 21.3°, 24.7°_{,} 25.0°, and 33.1°;
crystalline Form C is characterized by x-ray powder diffraction peaks at 20 angles (±0.2°) 11.5°, 15_{.}6°, 16.1°, 18.2°, 18.7°, 21.3°, 24.1°, and 25.2°;
crystalline Form D is characterized by x-ray powder diffraction peaks at 20 angles (± 0.2°) 4.5°, 13.2°, 16.1°, 16.9°, 18.1°; 21.7°, 22.4°, and 23.9°;
crystalline Form E is characterized by x-ray powder diffraction peaks at 20 angles (± 0.2°) 13.5°, 16.5°, 18.0°, 18.2°, 21.6°, 22.1°, 22.7°, and 23.7°;
crystalline Form F is characterized by x-ray powder diffraction peaks at 20 angles (± 0.2°) 11.3°, 15.3°, 15.8°, 18.0°, 19.0°, 19.9°, 22.8°, and 23.4°;
crystalline Form G is characterized by x-ray powder diffraction peaks at 20 angles (± 0.2°) 7.7°, 10.1°, 17.3°, 17.5°, 20.9°, 22.9°, 25.2°, and 25.7°;
crystalline Form H is characterized by x-ray powder diffraction peaks at 20 angles (± 0.2°) 10.8°, 19.9°, 20.0°, 20.8°, 21.4°, 21.6°, 24.5°, and 26.2°;
crystalline Form I is characterized by x-ray powder diffraction peaks at 20 angles (± 0.2°) 12.1°, 17.0°, 18.2°, 20.3°, 20.7°, 21.3°, 21.5°, and 25.1°;
crystalline Form J is characterized by x-ray powder diffraction peaks at 20 angles (± 0.2°) 12.8°, 14.2°, 17.4°, 20.0°, 21.9°, 22.1°, 22.5°, and 24.2°;
crystalline Form K is characterized by x-ray powder diffraction peaks at 20 angles (± 0.2°) 13.4°, 15.0°, 15.4°, 17.9°, 20.3°, 21.8°, 24.9°, and 27.6°;
crystalline Form L is characterized by x-ray powder diffraction peaks at 20 angles (± 0.2°) 7.4°, 13.7°, 14.4°, 14.8°, 22.7°, 22.9°, 23.4°, and 27.7°; and
crystalline Form M is characterized by x-ray powder diffraction peaks at 20 angles (± 0.2°) 12.9°, 15.7°, 15.9°, 17.8°_{.} 18.8°, 21.8°, 22.1°, and 24.5°.

7. The besylate salt, fumarate salt, gentisate salt, hydrochloride salt, maleate salt, malonate salt, phosphate salt, tartrate salt, or tosylate salt of claim 6, wherein
crystalline Form A is characterized by x-ray powder diffraction peaks at 2Θ angles (± 0.2°) 12.7°, 14.5°, 15.4°, 15.9°, 18.4°, 19.0°, 20.7°, 21.3°, 23.3°, 23.6°, 24.1°, and 24.5°;
crystalline Form B is characterized by x-ray powder diffraction peaks at 20 angles (± 0.2°) 4.1°, 8.2°, 10.8°, 14.8°, 15.3°, 17.8°, 20.5°, 21.3°, 21.7°, 24.7°, 25.0°, and 33.1°;
crystalline Form C is characterized by x-ray powder diffraction peaks at 20 angles (± 0.2°) 8.5°, 11.5°, 15_{.}6°, 16.1°, 17.8°, 18.2°, 18.7°, 21.0°, 21.3°, 24.1°, 25.2°, 27.8°, and 29.1°;
crystalline Form D is characterized by x-ray powder diffraction peaks at 20 angles (± 0.2°) 4.5°, 13.2°, 13.6°, 16.1°, 16.9°, 18.1°; 21.7°, 22.4°, 23.0°, 23.9°, 27.1°, and 27.3°;
crystalline Form E is characterized by x-ray powder diffraction peaks at 20 angles (± 0.2°) 11.8°, 13.5°, 16.5°, 18.0°, 18.2°, 21.6°, 22.1°, 22.7°, 23.7°, 24.1°, 25.8°, and 27.3°;
crystalline Form F is characterized by x-ray powder diffraction peaks at 20 angles (± 0.2°) 11.3°, 15.3°, 15.8°, 15.9°, 18.0°, 19.0°, 19.9°, 20.0°_{.} 22.8°, 23.4°, 23.6°, 25.6°, and 27.7°;
crystalline Form G is characterized by x-ray powder diffraction peaks at 20 angles (± 0.2°) 5.6°, 7.7°, 10.1°, 16.6°, 17.3°, 17.5°, 18.8°, 20.9°, 22.9°, 25.2°, and 25.7°;
crystalline Form H is characterized by x-ray powder diffraction peaks at 20 angles (± 0.2°) 10.8°, 15.8°, 16.5°, 18.3°, 19.4°, 19.9°, 20.0°_{.} 20.8°, 21.4°, 21.6°, 24.5°, and 26.2°;
crystalline Form I is characterized by x-ray powder diffraction peaks at 20 angles (± 0.2°) 12.1°, 16.1°, 17.0°, 18.2° (doublet), 20.3°, 20.7°, 21.3°, 21.5°, 22.0°, 23.4°, and 25.1°;
crystalline Form J is characterized by x-ray powder diffraction peaks at 20 angles (± 0.2°) 12.8°, 13.4°, 14.2°, 15.0°, 17.4°, 20.0°, 20.7°, 21.9°, 22.1°, 22.5°, 24.2°, and 24.7°;
crystalline Form K is characterized by x-ray powder diffraction peaks at 20 angles (± 0.2°) 12.6°, 12.9°, 13.4°, 15.0°, 15.4°, 16.4°, 17.9°, 18.7°, 20.3°, 21.8°, 24.9°, and 27.6°;
crystalline Form L is characterized by x-ray powder diffraction peaks at 20 angles (± 0.2°) 7.4°, 13.2°, 13.7°, 14.4°, 14.8°, 17.0°, 20.0°, 21.5°, 22.2°, 22.7°, 22.9°, 23.4°, and 27.7°; and crystalline Form M is characterized by x-ray powder diffraction peaks at 20 angles (± 0.2°) 12.9°, 13.5°, 15.7°, 15.9°, 17.8°_{.} 18.8°, 19.0°, 19.8°, 20.0°_{.} 21.8°, 22.1°, and 24.5°.

8. The besylate salt, fumarate salt, gentisate salt, hydrochloride salt, maleate salt, malonate salt, phosphate salt, tartrate salt, or tosylate salt of claim 7, wherein
crystalline Form A is characterized by an XRPD substantially similar to FIG. 1;
crystalline Form B is characterized by an XRPD substantially similar to FIG. 3;
crystalline Form C is characterized by an XRPD substantially similar to FIG. 5;
crystalline Form D is characterized by an XRPD substantially similar to FIG. 7;
crystalline Form E is characterized by an XRPD substantially similar to FIG. 9;
crystalline Form F is characterized by an XRPD substantially similar to FIG. 11;
crystalline Form G is characterized by an XRPD substantially similar to FIG. 13;
crystalline Form H is characterized by an XRPD substantially similar to FIG. 15;
crystalline Form I is characterized by an XRPD substantially similar to FIG. 17;
crystalline Form J is characterized by an XRPD substantially similar to FIG. 19;
crystalline Form K is characterized by an XRPD substantially similar to FIG. 21;
crystalline Form L is characterized by an XRPD substantially similar to FIG. 23; and crystalline Form M is characterized by an XRPD substantially similar to FIG. 25.

9-99. (canceled)

100. The crystalline Form A, B, C, D, E, F, G, H, I, J, K, L, or M of claim 4, wherein the crystalline form is at least 60% a single crystalline form, at least 70% a single crystalline form, at least 80% a single crystalline form, at least 90% a single crystalline form, at least 95% a single crystalline form, or at least 99% a single crystalline form by weight.

101. A pharmaceutical composition comprising the besylate salt, fumarate salt, gentisate salt, hydrochloride salt, maleate salt, malonate salt, phosphate salt, tartrate salt, or tosylate salt of claim 1; and a pharmaceutically acceptable carrier.

102. The composition of claim 101, wherein the composition is a tablet composition.

103. The composition of claim 102, wherein the carrier is selected from one or more of microcrystalline cellulose, mannitol, croscarmellose sodium, and sodium stearyl fumarate.

104. The tablet composition of claim 102, wherein the composition comprises about 5.7 to about 5.9 mg, about 23.4 to about 23.6 mg, or about 58.7 to about 58.9 mg crystalline Form A, B, C, D, E, F, G, H, I, J, K, L, or M as recited in any one of claims (Currently Amended) 4-8, 14-23, 27-36, 42-51, 55-59, 63-67, 73-82, 87-91, or 95-99; 62% w/w (±2%) microcrystalline cellulose; 23% w/w (±2%) mannitol, 3% w/w (±2%) croscarmellose sodium, and 2% w/w (±2%) stearyl fumarate.

105. A method of treating Pyruvate Kinase Deficiency (PKD), sickle cell disease (SCD), thalassemia, or hemolytic anemia in a subject in need thereof, comprising administering to the subject an effective amount of the besylate salt, fumarate salt, gentisate salt, hydrochloride salt, maleate salt, malonate salt, phosphate salt, tartrate salt, or tosylate salt of claim 1 .

106-113. (canceled)
